(19) United States
(12) Patent Application Publication
(10) Pub. No.: US 2019/0161555 A1
(43) Pub. Date: May 30, 2019

(54) Bispecific Antibodies Directed Against OX40 and a Tumor-Associated Antigen

(71) Applicant: ALLIGATOR BIOSCIENCE AB, Lund (SE)

(72) Inventors: Peter Ellmark, Lund (SE); Per Norlen, Lund (SE); Jessica Petersson, Lund (SE); Sara Fritzell, Lund (SE); Eva Dahlen, Lund (SE)

(21) Appl. No.: 16/312,374

(22) PCT Filed: Jun. 30, 2017
(86) PCT No.: PCT/EP2017/066350

§ 371 Date: Dec. 21, 2018

(30) Foreign Application Priority Data
Jul. 1, 2016 (GB) 1611530.5

(51) Int. Cl. C07K 16/28; A61P 35/00; C12N 15/85; C12N 15/62; C07K 16/30

(52) U.S. Cl. CPC C07K 16/2878; A61P 35/00; C12N 15/85; C12N 15/62; C07K 16/30; A61K 9/0019; C07K 2317/31; C07K 2317/626; C12N 2015/8518; C07K 2317/73; C07K 2317/75; C07K 2317/52

(57) ABSTRACT

The invention provides bispecific polypeptides comprising a first binding domain, designated B1, which is capable of binding specifically to OX40, and a second binding domain, designated B2, which is capable of specifically binding to a tumour cell-associated antigen. Also provided are pharmaceutical compositions of such bispecific polypeptides and uses of the same in medicine.

### FIELD OF INVENTION

The present invention relates to novel bispecific polypeptides, such as antibodies, and their use in the treatment of cancers.

### BACKGROUND

Immunotherapy of Cancer

Cancer is a leading cause of premature deaths in the developed world. Immunotherapy of cancer aims to mount an effective immune response against tumour cells. This may be achieved by, for example, breaking tolerance against tumour antigen, augmenting anti-tumor immune responses, and stimulating local cytokine responses at the tumor site. The key effector cell of a long lasting anti-tumor immune response is the activated tumor specific effector T cell. Potent expansion of activated tumour-specific effector T cells can redirect the immune response towards the tumor. In this context, various immunosuppressive mechanisms induced by the tumor microenvironment suppress the activity of effector T cells. Several immunosuppressive mediators are expressed by the tumor cells. Such mediators inhibit T cell activation, either directly, or indirectly by inducing e.g. regulatory T cells (Treg) or myeloid-derived suppressor cells. Depleting, inhibiting, reverting or inactivating such regulatory cells may therefore provide anti-tumor effects and revert the immune suppression in the tumor microenvironment. Further, incomplete activation of effector T cells by, for example, dendritic cells can result in sub-optimally activated or anergic T cells, resulting in an inefficient anti-tumor response. In contrast, adequate induction by dendritic cells can generate a potent expansion of activated effector T cells, redirecting the immune response towards the tumor. In addition, Natural killer (NK) cells play an important role in tumor immunology by attacking tumor cells with down-regulated human leukocyte antigen (HLA) expression and by inducing antibody dependent cellular cytotoxicity (ADCC). Stimulation of NK cells may thus also reduce tumor growth.

Tumour-Associated Antigens

Tumor-associated antigens (TAA) are cell surface proteins selectively expressed on tumor cells. The term tumor-associated indicates that TAA are not completely tumor-specific, but are rather over-expressed on the tumor. A vast number of TAA have been described and used in various therapeutic rationales, including monoclonal antibodies, T cell redirecting therapies with TAA-CD3 bispecific antibodies, immunocytokines and antibody drug conjugates. Some well-studied TAA include the EGFR family molecules (HER2, HER3 and EGFR/HER1), VEGFR, EpCAM, CEA, PSA, PSMA, EphA2, gp100, GD2, MUC1, CD20, CD19, CD22 and CD33, summarized in (Cheever et al., 2009).

5T4 (also designated trophoblast glycoprotein, TPBG, M6P1 and Waif1) is a well-defined TAA originally identified by Professor Peter Stern, University of Manchester (Hole and Stern, 1988). It is an oncofetal antigen expressed in a high proportion of patients in a variety of malignancies, including non-small cell lung, renal, pancreas, prostate, breast, colorectal, gastric, ovarian and cervix cancers as well as in acute lymphocytic leukemia, and has also been shown to be expressed in tumor-initiating cells (Castro et al., 2012; Damelin et al., 2011; Elkord et al., 2009; Southall et al., 1990).

5T4 expression is tumor-selective, with no or low expression in most normal tissues. In non-malignant tissue, 5T4 is mainly expressed in the placenta (trophoblast and amniotic epithelium) and at low levels in some specialised epithelia (Hole and Stern, 1988), as well as low at levels in other normal tissues (see US 2010/0021483). However, although low levels have been detected in some healthy tissue, the safety risk associated with this is considered low since expression levels in the tumor are considerably higher. This is supported by the fact that the phase III clinical programs, ANYARA and TroVax targeting 5T4 did not report severe 5T4-related toxicities.

Data from Stern et al. demonstrate that 5T4 regulates the functional activity of CXCR4 (Castro et al., 2012; Southgate et al., 2010). 5T4 binding antibodies or 5T4 knock-down resulted in inhibition of CXCR4-mediated cellular migration. The CXCR4 pathway is involved in tumor growth and metastasis. Therefore, targeting 5T4 in a CXCR4 inhibitory manner is likely to reduce tumor growth and/or spread.

EpCAM (Alternative names: BerEp4, CD326, CO-171A, 17-1A, EpCAM/Ep-AM, ESA, EGP, EGP-2, EGP34, EGP40, GA733-2, HEA125, KSA, KS1/4, MH99, MK-1, MOC31, TROP 1, VU-1D9, 323/A3 is overexpressed on malignant carcinomas (Patriarca et al., 2012) (Yao et al. 2013) (Lund et al., 2014) (Schnell et al., 2013). EpCAM is a type I, transmembrane, 39-42 kDa glycoprotein that functions as a epithelial-specific intercellular adhesion molecule (Patriarca et al., 2012).

EGFR is amplified and dysregulated on several cancer types. EGFR is expressed in different conformations which are functionally active or inactive, and can be discriminated by specific antibodies. EGFR regulates cellular growth, apoptosis, migration, adhesion and differentiation (Yarden, 2001; Yarden and Sliwkowski, 2001). Overexpression or continuous signalling through this receptor is common in carcinomas.

HER2, also known as CD340 (cluster of differentiation 340), proto-oncogene Neu, Erbb2 (rodent), or ERBB2, is amplified and dysregulated in many tumor types, in particular in breast cancer (Yarden, 2001). Over-expression of this oncogene has been shown to play an important role in the development and progression of cancer.

OX40

OX40 (otherwise known as CD134 or TNFRSF4) is a member of the TNFR family that is expressed mainly on activated T cells (mostly CD4+ effector T cells, but also CD8+ effector T-cells and regulatory T cells (Tregs)). In mice the expression is constitutive on Tregs, but not in humans. OX40 expression typically occurs within 24 hours of activation (T cell receptor engagement) and peaks after 48-72 hours. OX40 stimulation is important for the survival and proliferation of activated T cells. The only known ligand for OX40 is OX40L, which is mainly expressed on antigen presenting cells, such as dendritic cells and B cells, typically following their activation. The net result of OX40-mediated T cell activation is the induction of a TH1 effector T cell activation profile and a reduction in the activity and/or numbers of Treg cells e.g. via ADCC or ADCP. Overall these effects may contribute to anti-tumor immunity. OX40 is overexpressed on regulatory T cells in many solid tumors, such as melanoma, lung cancer and renal cancer.

OX40 agonist treatment of tumor models in mice has been shown to result in anti-tumor effects and cure of several different cancer forms, including melanoma, glioma, sarcoma, prostate, colon and renal cancers. The data is consistent with a tumor specific T-cell response, involving both CD4+ and CD8+ T cells, similar to the effect seen with CD40 agonist treatments. Addition of IL-12 and other cytokines, and combination with other immunomodulators and chemo/radiotherapy, has been shown to improve the therapeutic effect of OX40 agonist treatment. A clinical phase I study testing the mouse anti-human OX40 Clone 9B12 in late stage patients that had failed all other therapy has been conducted at the Providence Cancer Centre. The antibody was well-tolerated. Tumor shrinkage and an increase in CD4+ and CD8+ T cell proliferation were observed. The low toxicity may be caused by low half-life and anti-drug antibodies (the antibody was a mouse antibody), but also by the relatively low expression levels of OX40 on non-activated T cells. The anti-tumor effect with this antibody was modest.

Despite progress in the development of immunotherapies for the treatment of various cancers over the last decade, there remains a need for new and efficacious agents.

Accordingly, the present invention seeks to provide improved polypeptide-based therapies for the treatment of cancer.

### SUMMARY OF INVENTION

A first aspect of the invention provides a bispecific polypeptide comprising a first binding domain, designated B1, which is capable of binding specifically to OX40, and a second binding domain, designated B2, which is capable of specifically binding to a tumour cell-associated antigen.

Such bispecific compounds comprising one tumor-targeting moiety, e.g. a 5T4 binder, and one immune-activating moiety, e.g. an OX40 agonist, can be used to establish a highly effective and safe cancer immunotherapy.

Various types of tumor-localizing immunotherapeutic molecules, such as immunocytokines and bispecific antibodies have shown beneficial immune activation and inhibition of tumor growth in preclinical studies as well as in the clinic (reviewed in Kiefer and Neri, 2016).

To avoid affecting part of the immune system not relevant for inducing tumor immunity and avoid systemic toxicity by OX40 activating agents, yet obtain high efficacy in the tumor area, the designs of the molecular format of an OX40 agonist may be optimised. For example, a good efficacy/safety profile can be obtained by a TAA-OX40 bispecific antibody that requires crosslinking by binding to the TAA for OX40 activation to occur. Then, pre-activated, OX40-expressing T cells residing in the tumour will preferentially be activated, whereas OX40-expressing cells in other tissues will not. This would allow focused activation of the relevant, tumour-specific T cells while limiting toxicity induced by generalised OX40 activation (‘activation’ in this context being a net immune activation that results in a tumor-directed T cell response, for example by down-regulation of Tregs suppressive function and/or upregulation of effector T cell function).

The clinical progress with immunocytokines has so far not been impressive and the side effects still remain since the tumor-binding entity only confers limited tumor localization, with the bulk of the immunocytokine ending up in other compartments. Bispecific antibodies that restrict the activity to the tumor as described in this invention would provide a clear advantage over immunocytokines since they are inactive in the absence of tumors.

Further, the bispecific polypeptides of the invention provide a distinct advantage over bispecific antibodies targeting CD3. CD3-targeting bispecific molecules use T cells as effector cells and are capable of activating T cells independent of TAA binding. Thus they do not activate tumor specific T-cells in particular. The resulting anti-tumor effects are therefore not likely to generate a long lasting anti-tumor immunity. In addition, since CD3 is expressed on all T cells, systemic T cell activation is associated with toxicity issues. In contrast, the bispecific antibodies of the invention have the potential to selectively activate tumor specific T-cells and generate a long lasting tumour immunity.

Structure of Bispecific Polypeptide

A “polypeptide” is used herein in its broadest sense to refer to a compound of two or more subunit amino acids, amino acid analogs, or other peptidomimetics. The term “polypeptide” thus includes short peptide sequences and also longer polypeptides and proteins. As used herein, the term “amino acid” refers to either natural and/or unnatural or synthetic amino acids, including both D or L optical isomers, and amino acid analogs and peptidomimetics.

The term “bispecific” as used herein means the polypeptide is capable of specifically binding at least two target entities.

In one preferred embodiment, the polypeptide is a bispecific antibody (numerous examples of which are described in detail below).

Thus, the first and/or second binding domains may be selected from the group consisting of antibodies and antigen-binding fragments thereof.

By “an antibody or an antigen-binding fragment thereof” we include substantially intact antibody molecules, as well as chimaeric antibodies, humanised antibodies, isolated human antibodies, single chain antibodies, bispecific antibodies, antibody heavy chains, antibody light chains, homodimers and heterodimers of antibody heavy and/or light chains, and antigen-binding fragments and derivatives of the same. Suitable antigen-binding fragments and derivatives include Fv fragments (e.g. single chain Fv and disulphide-bonded Fv), Fab-like fragments (e.g. Fab fragments, Fab′ fragments and F(ab)2 fragments), single variable domains (e.g. VH and VL domains) and single domain antibodies (dAbs, including single and dual formats [i.e. dAb-linker-dAb], and nanobodies). The potential advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli, thus allowing the facile production of large amounts of the said fragments.

In one embodiment, the antigen-binding fragment is selected from the group consisting of: Fv fragments (such as a single chain Fv fragment, or a disulphide-bonded Fv fragment), Fab-like fragments (such as a Fab fragment; a Fab′ fragment or a F(ab)₂ fragment) and single domain antibodies.

The phrase “an antibody or an antigen-binding fragment thereof” is also intended to encompass antibody mimics (for example, non-antibody scaffold structures that have a high degree of stability yet allow variability to be introduced at certain positions). Those skilled in the art of biochemistry will be familiar with many such molecules, as discussed in Gebauer & Skerra, 2009 (the disclosures of which are incorporated herein by reference). Exemplary antibody mimics include: affibodies (also called Trinectins; Nygren, 2008, FEBS J, 275, 2668-2676); CTLDs (also called Tetranectins; Innovations Pharmac. Technol. (2006), 27-30); adnectins (also called monobodies; Meth. Mol. Biol., 352 (2007), 95-109); anticalins (Drug Discovery Today (2005), 10, 23-33); DARPins (ankyrins; Nat. Biotechnol. (2004), 22, 575-582); avimers (Nat. Biotechnol. (2005), 23, 1556-1561); microbodies (FEBS J, (2007), 274, 86-95); peptide aptamers (Expert. Opin. Biol. Ther. (2005), 5, 783-797); Kunitz domains (J. Pharmacol. Exp. Ther. (2006) 318, 803-809); affilins (Trends. Biotechnol. (2005), 23, 514-522); affimers (Avacta Life Sciences, Wetherby, UK).

Also included within the scope of the invention are chimaeric T-cell receptors (also known as chimaeric T cell receptors, chimaeric immunoreceptors, and chimaeric antigen receptors or CARs) (see Pule et al., 2003, the disclosures of which are incorporated herein by reference). These are engineered receptors, which graft an arbitrary specificity onto an immune effector cell. Typically, CARs are used to graft the specificity of a monoclonal antibody onto a T cell; with transfer of their coding sequence facilitated by retroviral vectors. The most common form of such molecules is fusions comprising a single-chain variable fragment (scFv) derived from a monoclonal antibody fused to CD3-zeta transmembrane and endodomain. When T cells express this fusion molecule, they recognize and kill target cells that express the transferred monoclonal antibody specificity.

Persons skilled in the art will further appreciate that the invention also encompasses modified versions of antibodies and antigen-binding fragments thereof, whether existing now or in the future, e.g. modified by the covalent attachment of polyethylene glycol or another suitable polymer (see below).

Methods of generating antibodies and antibody fragments are well known in the art. For example, antibodies may be generated via any one of several methods which employ induction of in vivo production of antibody molecules, screening of immunoglobulin libraries (Orlandi. et al, 1989; Winter et al., 1991, the disclosures of which are incorporated herein by reference) or generation of monoclonal antibody molecules by cell lines in culture. These include, but are not limited to, the hybridoma technique, the human B-cell hybridoma technique, and the Epstein-Barr virus (EBV)-hybridoma technique (Kohler et al., 1975, Kozbor et al., 1985; Cote et al., 1983; Cole et al., 1984., the disclosures of which are incorporated herein by reference).

Suitable methods for the production of monoclonal antibodies are also disclosed in “Monoclonal Antibodies: A manual of techniques”, H Zola (CRC Press, 1988, the disclosures of which are incorporated herein by reference) and in “Monoclonal Hybridoma Antibodies: Techniques and Applications”, J G R Hurrell (CRC Press, 1982, the disclosures of which are incorporated herein by reference).

Likewise, antibody fragments can be obtained using methods well known in the art (see, for example, Harlow & Lane, 1988, “Antibodies: A Laboratory Manual”, Cold Spring Harbor Laboratory, New York, the disclosures of which are incorporated herein by reference). For example, antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Alternatively, antibody fragments can be obtained by pepsin or papain digestion of whole antibodies by conventional methods.

It will be appreciated by persons skilled in the art that for human therapy or diagnostics, human or humanised antibodies are preferably used. Humanised forms of non-human (e.g. murine) antibodies are genetically engineered chimaeric antibodies or antibody fragments having preferably minimal-portions derived from non-human antibodies. Humanised antibodies include antibodies in which complementary determining regions of a human antibody (recipient antibody) are replaced by residues from a complementary determining region of a non-human species (donor antibody) such as mouse, rat or rabbit having the desired functionality. In some instances, Fv framework residues of the human antibody are replaced by corresponding non-human residues. Humanised antibodies may also comprise residues which are found neither in the recipient antibody nor in the imported complementarity determining region or framework sequences. In general, the humanised antibody will comprise substantially all of at least one, and typically two, variable domains, in which all or substantially all of the complementarity determining regions correspond to those of a non-human antibody and all, or substantially all, of the framework regions correspond to those of a relevant human consensus sequence. Humanised antibodies optimally also include at least a portion of an antibody constant region, such as an Fc region, typically derived from a human antibody (see, for example, Jones et al., 1986, Riechmann et al., 1988, Presta, 1992, the disclosures of which are incorporated herein by reference). Methods for humanising non-human antibodies are well known in the art. Generally, the humanised antibody has one or more amino acid residues introduced into it from a source which is non-human. These non-human amino acid residues, often referred to as imported residues, are typically taken from an imported variable domain. Humanisation can be essentially performed as described (see, for example, Jones et al., 1986, Reichmann et al., 1988, Verhoeyen et al., 1988, U.S. Pat. No. 4,816,567, the disclosures of which are incorporated herein by reference) by substituting human complementarity determining regions with corresponding rodent complementarity determining regions. Accordingly, such humanised antibodies are chimaeric antibodies, wherein substantially less than an intact human variable domain has been substituted by the corresponding sequence from a non-human species. In practice, humanised antibodies may be typically human antibodies in which some complementarity determining region residues and possibly some framework residues are substituted by residues from analogous sites in rodent antibodies.

Human antibodies can also be identified using various techniques known in the art, including phage display libraries (see, for example, Hoogenboom & Winter, 1991, Marks et al., 1991, Cole et al., 1985, Boerner et al., 1991, the disclosures of which are incorporated herein by reference).

It will be appreciated by persons skilled in the art that the bispecific polypeptides, e.g. antibodies, of the present invention may be of any suitable structural format.

Thus, in exemplary embodiments of the bispecific antibodies of the invention:
(a) binding domain B1 and/or binding domain B2 is an intact IgG antibody (or, together, form an intact IgG antibody);
(b) binding domain B1 and/or binding domain B2 is an Fv fragment (e.g. an scFv);
(c) binding domain B1 and/or binding domain B2 is a Fab fragment; and/or
(d) binding domain B1 and/or binding domain B2 is a single domain antibody (e.g. domain antibodies and nanobodies).

It will be appreciated by persons skilled in the art that the bispecific antibody may comprise a human Fc region, or a variant of a said region, where the region is an IgG1, IgG2, IgG3 or IgG4 region, preferably an IgG1 or IgG4 region.

Engineering the Fc region of a therapeutic monoclonal antibody or Fc fusion protein allows the generation of molecules that are better suited to the pharmacology activity required of them (Strohl, 2009, the disclosures of which are incorporated herein by reference).

(a) Engineered Fc Regions for Increased Half-Life

One approach to improve the efficacy of a therapeutic antibody is to increase its serum persistence, thereby allowing higher circulating levels, less frequent administration and reduced doses.

The half-life of an IgG depends on its pH-dependent binding to the neonatal receptor FcRn. FcRn, which is expressed on the surface of endothelial cells, binds the IgG in a pH-dependent manner and protects it from degradation.

Some antibodies that selectively bind the FcRn at pH 6.0, but not pH 7.4, exhibit a higher half-life in a variety of animal models.

Several mutations located at the interface between the CH2 and CH3 domains, such as T250Q/M428L (Hinton et al., 2004, the disclosures of which are incorporated herein by reference) and M252Y/S254T/T256E+H433K/N434F (Vaccaro et al., 2005, the disclosures of which are incorporated herein by reference), have been shown to increase the binding affinity to FcRn and the half-life of IgG1 in vivo.

(b) Engineered Fc Regions for Altered Effector Function

To ensure lack of OX40 activation in the absence of the tumour antigen, the Fc portion of the bispecific antibody should bind with no or very low affinity to FcγR, since FcγR-mediated crosslinking of an OX40 antibody may induce activation. By “very low affinity” we include that the Fc portion exhibits at least 10 times reduced affinity to FcγRI, FcgRII and III compared to wild-type IgG1, as determined by the concentration where half maximal binding is achieved in flow cytometric analysis of FcγR expressing cells (Hezareh et al., 2001) or by FcγR ELISA (Shields et al., 2001).

Another factor to take into account is that engagement of FcγR's may also induce antibody-dependent cellular cytotoxicity (ADCC), antibody-dependent cellular phagocytosis (ADCP) and complement-dependent cytotoxicity (CDC) of cells coated with antibodies. Thus, to ensure tumor-dependent OX40 activation as well as to avoid depletion of OX40-expressing, tumor-reactive T effector cells, the isotype of a TAA-OX40 bispecific antibody should preferably be silent.

The four human IgG isotypes bind the activating Fcγ receptors (FcγRI, FcγRIIa, FcγRIIIa), the inhibitory FcγRIIb receptor, and the first component of complement (C1q) with different affinities, yielding very different effector functions (Bruhns et al., 2009, the disclosures of which are incorporated herein by reference). IgG1 molecules have the highest affinity and capacity to induce effector functions, whereas IgG2, IgG3 and IgG4 are less effective (Bruhns, 2012; Hogarth and Pietersz, 2012; Stewart et al., 2014) (Wang et al. 2015; Vidarson et al. 2014). In addition, certain mutations in the Fc region of IgG1 dramatically reduces FcγR affinity and effector function while retaining neonatal FcR (FcRn) interaction (Ju and Jung, 2014; Leabman et al., 2013; Oganesyan et al., 2008; Sazinsky et al., 2008).

The most widely used IgG1 mutants are N297A alone or in combination with D265A, as well as mutations at positions L234 and L235, including the so-called “LALA” double mutant L234A/L235A. Another position described to further silence IgG1 by mutation is P329 (see US 2012/0251531).

Thus, choosing a mutated IgG1 format with low effector function but retained binding to FcRn may result in a bispecific antibody with 5T4-dependent activation of CD137, and exhibiting a favorable efficacy/safety profile and good PK properties.

Advantageously, the polypeptide is incapable of inducing antibody dependent cell cytotoxicity (ADCC), antibody-dependent cellular phagocytosis (ADCP), and/or complement-dependent cytotoxicity (CDC). By “incapable” we include that the ability of the polypeptide to induce ADCC, etc., is at least 10-fold lower than compared to wild-type IgG1 as shown by e.g. monocyte-dependent ADCC or CDC assays described by Hezareh et al. 2001.

In one embodiment, the Fc region may be a variant of a human IgG1 Fc region comprising a mutation at one or more of the following positions:
L234, L235, P239, D265, N297 and/or P329.

Advantageously, alanine may be present at the mutated positions(s).

Optionally, the IgG1 variant may be a variant of a human IgG1 Fc region comprising mutations L234A and L235A (i.e. the LALA double mutant; see SEQ ID NO: 103).

It will be appreciated by persons skilled in the art that the bispecific polypeptides of the invention may be of several different structural formats (for example, see Chan & Carter, 2016, the disclosures of which are incorporated herein by reference).

In exemplary embodiments, the bispecific antibody is selected from the groups consisting of:
(a) bivalent bispecific antibodies, such as IgG-scFv bispecific antibodies (for example, wherein B1 is an intact IgG and B2 is an scFv attached to B1 at the N-terminus of a light chain and/or at the C-terminus of a light chain and/or at the N-terminus of a heavy chain and/or at the C-terminus of a heavy chain of the IgG, or vice versa);
(b) monovalent bispecific antibodies, such as a DuoBody® (Genmab AS, Copenhagen, Denmark) or ‘knob-in-hole’ bispecific antibody (for example, an scFv-KIH, scFv-KIH^{r}, a BiTE-KIH or a BiTE-KIH^{r} (see Xu et al., 2015, mAbs 7(1):231-242);
(c) scFv₂-Fc bispecific antibodies (such as ADAPTIR™ bispecific antibodies from Emergent Biosolutions Inc);
(d) BiTE/scFv₂ bispecific antibodies;
(e) DVD-Ig bispecific antibodies;
(f) DART-based bispecific antibodies (for example, DART₂-Fc, DART₂-Fc or DART);
(g) DNL-Fab₃ bispecific antibodies; and
(h) scFv-HSA-scFv bispecific antibodies.

For example, the bispecific antibody may be an IgG-scFv antibody. The IgG-scFv antibody may be in either VH-VL or VL-VH orientation. In one embodiment, the scFv may be stabilised by a S—S bridge between VH and VL.

In one embodiment, binding domain B1 and binding domain B2 are fused directly to each other.

In an alternative embodiment, binding domain B1 and binding domain B2 are joined via a polypeptide linker. For example, a polypeptide linker may be a short linker peptide between about 10 to about 25 amino acids. The linker is usually rich in glycine for flexibility, as well as serine or threonine for solubility, and can either connect the N-terminus of the VH with the C-terminus of the VL, or vice versa.

Thus, the linker may be selected from the group consisting of the amino acid sequence SGGGGSGGGGS (SEQ ID NO: 104), SGGGGSGGGGSAP (SEQ ID NO: 105), NFSQP (SEQ ID NO: 106), KRTVA (SEQ ID NO: 107), GGGSGGGG (SEQ ID NO: 108), GGGGSGGGGS, (SEQ ID NO: 109), GGGGSGGGGSGGGGS (SEQ ID NO: 110), GSTSGSGKPGSGEGSTKG (SEQ ID NO: 116) (Whitlow et al. 1993) THTCPPCPEPKSSDK (SEQ ID NO: 117), GGGS (SEQ ID NO: 118), EAAKEAAKGGGGS (SEQ ID NO: 119), EAAKEAAK (SEQ ID NO: 120), or (SG)m, where m=1 to 7.

In a preferred embodiment, the linker may be selected from the group consisting of: SEQ ID NO: 108, SEQ ID NO: 110 and SEQ ID NO: 116.

The term “amino acid” as used herein includes the standard twenty genetically-encoded amino acids and their corresponding stereoisomers in the ‘D’ form (as compared to the natural ‘L’ form), omega-amino acids other naturally-occurring amino acids, unconventional amino acids (e.g. α,α-disubstituted amino acids, N-alkyl amino acids, etc.) and chemically derivatised amino acids (see below).

When an amino acid is being specifically enumerated, such as “alanine” or “Ala” or “A”, the term refers to both L-alanine and D-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid.

In one embodiment, the antibody polypeptides as defined herein comprise or consist of L-amino acids.

It will be appreciated by persons skilled in the art that the antibody polypeptides of the invention may comprise or consist of one or more amino acids which have been modified or derivatised.

Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

It will be further appreciated by persons skilled in the art that peptidomimetic compounds may also be useful. The term ‘peptidomimetic’ refers to a compound that mimics the conformation and desirable features of a particular peptide as a therapeutic agent.

For example, the said polypeptide includes not only molecules in which amino acid residues are joined by peptide (—CO—NH—) linkages but also molecules in which the peptide bond is reversed. Such retro-inverso peptidomimetics may be made using methods known in the art, for example such as those described in Meziere et al. (1997), which is incorporated herein by reference. This approach involves making pseudo-peptides containing changes involving the backbone, and not the orientation of side chains. Retro-inverse peptides, which contain NH—CO bonds instead of CO—NH peptide bonds, are much more resistant to proteolysis. Alternatively, the said polypeptide may be a peptidomimetic compound wherein one or more of the amino acid residues are linked by a -y(CH₂NH)— bond in place of the conventional amide linkage.

In a further alternative, the peptide bond may be dispensed with altogether provided that an appropriate linker moiety which retains the spacing between the carbon atoms of the amino acid residues is used; it may be advantageous for the linker moiety to have substantially the same charge distribution and substantially the same planarity as a peptide bond.

It will also be appreciated that the said polypeptide may conveniently be blocked at its N- or C-terminus so as to help reduce susceptibility to exo-proteolytic digestion.

A variety of un-coded or modified amino acids such as D-amino acids and N-methyl amino acids have also been used to modify mammalian peptides. In addition, a presumed bioactive conformation may be stabilised by a covalent modification, such as cyclisation or by incorporation of lactam or other types of bridges, for example see Veber et al., 1978 and Thursell et al., 1983, which are incorporated herein by reference.

In one embodiment, the bispecific polypeptide of the invention is capable of inducing tumour immunity. This can be tested in vitro in T cell activation assays, e.g. by measuring IL-2 and IFNγ production. Activation of effector T cells would indicate that a tumour specific T cell response can be achieved in vivo. Further, an anti-tumour response in an in vivo model, such as a mouse model would imply that a successful immune response towards the tumour has been achieved.

Thus, the bispecific polypeptide may modulate the activity of a target immune system cell, wherein said modulation is an increase or decrease in the activity of said cell. Such cells include T cells, dendritic cells and natural killer cells.

The immune system cell is typically a T cell. Thus, the antibody may increase the activity of a CD4+ or CD8+ effector T cell, or may decrease the activity of a regulatory T cell (Treg). In either case, the net effect of the antibody will be an increase in the activity of effector T cells, particularly CD8+ effector T cells. Methods for determining a change in the activity of effector T cells are well known and include, for example, measuring for an increase in the level of T cell cytokine production (e.g. IFN-γ or IL-2) or an increase in T cell proliferation in the presence of the antibody relative to the level of T cell cytokine production and/or T cell proliferation in the presence of a control. Assays for cell proliferation and/or cytokine production are well known.

For example, the polypeptide may be capable of inducing:
(a) activation of cytotoxic T cells, i.e. CD8+ T cells;
(b) activation of helper T cells, i.e. CD4⁺ T cells;
(c) reprogramming of Tregs into effector T cells

The polypeptide or binding domains of the invention can also be characterised and defined by their binding abilities. Standard assays to evaluate the binding ability of ligands towards targets are well known in the art, including for example, ELISAs, Western blots, RIAs, and flow cytometry analysis. The binding kinetics (e.g., binding affinity) of the polypeptide also can be assessed by standard assays known in the art, such as by Surface Plasmon Resonance analysis (SPR).

The terms “binding activity” and “binding affinity” are intended to refer to the tendency of a polypeptide molecule to bind or not to bind to a target. Binding affinity may be quantified by determining the dissociation constant (Kd) for a polypeptide and its target. A lower Kd is indicative of a higher affinity for a target. Similarly, the specificity of binding of a polypeptide to its target may be defined in terms of the comparative dissociation constants (Kd) of the polypeptide for its target as compared to the dissociation constant with respect to the polypeptide and another, non-target molecule.

The value of this dissociation constant can be determined directly by well-known methods, and can be computed even for complex mixtures by methods such as those, for example, set forth in Caceci et al., 1984 (the disclosures of which are incorporated herein by reference). For example, the Kd may be established using a double-filter nitrocellulose filter binding assay such as that disclosed by Wong & Lohman, 1993. Other standard assays to evaluate the binding ability of ligands such as antibodies towards targets are known in the art, including for example, ELISAs, Western blots, RIAs, and flow cytometry analysis. The binding kinetics (e.g., binding affinity) of the antibody also can be assessed by standard assays known in the art, such as by Biacore™ system analysis.

A competitive binding assay can be conducted in which the binding of the antibody to the target is compared to the binding of the target by another, known ligand of that target, such as another antibody. The concentration at which 50% inhibition occurs is known as the Ki. Under ideal conditions, the Ki is equivalent to Kd. The Ki value will never be less than the Kd, so measurement of Ki can conveniently be substituted to provide an upper limit for Kd.

Alternative measures of binding affinity include EC50 or IC50. In this context EC50 indicates the concentration at which a polypeptide achieves 50% of its maximum binding to a fixed quantity of target. IC50 indicates the concentration at which a polypeptide inhibits 50% of the maximum binding of a fixed quantity of competitor to a fixed quantity of target. In both cases, a lower level of EC50 or IC50 indicates a higher affinity for a target. The EC50 and IC50 values of a ligand for its target can both be determined by well-known methods, for example ELISA. Suitable assays to assess the EC50 and IC50 of polypeptides are set out in the Examples.

A polypeptide of the invention is preferably capable of binding to its target with an affinity that is at least two-fold, 10-fold, 50-fold, 100-fold or greater than its affinity for binding to another non-target molecule.

OX40 Binding Domains

The bispecific polypeptides of the invention comprise a binding domain (B1) which is capable of specifically binding to OX40.

Binding domain B1 specifically binds to OX40, i.e. it binds to OX40 but does not bind, or binds at a lower affinity, to other molecules. The term OX40 as used herein typically refers to human OX40. The sequence of human OX40 is set out in GenBank: NP_003318.1. Binding domain B1 may have some binding affinity for OX40 from other mammals, such as OX40 from a non-human primate (for example Macaca fascicularis (cynomolgus monkey), Macaca mulatta). Binding domain B1 preferably does not bind to murine OX40 and/or does not bind to other human TNFR superfamily members, for example human CD137 or CD40.

Advantageously, binding domain B1 binds to human OX40 with a K_{D} of less than 50×10⁻¹⁰M or less than 25×10⁻¹⁰M, more preferably less than 10, 9, 8, 7, or 6×10⁻¹⁰M, most preferably less than 5×10⁻¹⁰M.

For example, binding domain B1 preferably does not bind to murine OX40 or any other TNFR superfamily member, such as CD137 or CD40. Therefore, typically, the Kd for the binding domain with respect to human OX40 will be 2-fold, preferably 5-fold, more preferably 10-fold less than Kd with respect to the other, non-target molecule, such as murine OX40, other TNFR superfamily members, or any other unrelated material or accompanying material in the environment. More preferably, the Kd will be 50-fold less, even more preferably 100-fold less, and yet more preferably 200-fold less.

Binding domain B1 is preferably capable of binding to its target with an affinity that is at least two-fold, 10-fold, 50-fold, 100-fold or greater than its affinity for binding to another non-target molecule.

In summary therefore, binding domain B1 preferably exhibits at least one of the following functional characteristics:
I. binding to human OX40 with a K_{D} value which is less than 10×10⁻¹⁰ M;
II. does not bind to murine OX40;
III. does not bind to other human TNFR superfamily members, for example human CD137 or CD40.

The binding domain B1 is specific for OX40, typically human OX40 and may comprise any one, two, three, four, five or all six of the following:
(a) a heavy chain CDR1 sequence which is 8 amino acids in length and comprises the consensus sequence: “G, F, T, F, G/Y/S, G/Y/S, Y/S, Y/S/A”;
(b) a heavy chain CDR2 sequence which is 8 amino acids in length and comprises the consensus sequence: “I, G/Y/S/T, G/S/Y, S/Y, G/S/Y, G/S/Y, G/S/Y, T”; (c) a heavy chain CDR3 sequence which is 9 to 17 amino acids in length and which comprises the consensus sequence of: “A, R, G/Y/S/H, G/Y/F/V/D, G/Y/P/F, -/H/S, -/N/D/H, -Y/G, -/Y, -/Y, -/W/A/V, -/A/Y, -/D/A/Y/G/H/N, Y/S/W/A/T, L/M/I/F, D, Y”. Preferred heavy chain CDR3 sequences within this definition include a CDR3 sequence of 10 amino acids in length which comprises the consensus sequence “A, R, Y/H, D, Y, A/Y/G, S/W/A, M/L, D, Y” or a CDR3 sequence of 11 amino acids in length which comprises the consensus sequence “A, R, G/Y, V/F/Y, P, H, G/Y/H, Y, F/I, D, Y”;
(d) a light chain CDR1 sequence which consists of the sequence: “Q, S, I, S, S, Y”;
(e) a light chain CDR2 sequence which consists of the sequence: “A, A, S”;
(f) a light chain CDR3 sequence which is 8 to 10 amino acids in length and comprises the consensus sequence: “Q,Q, S/Y/G, -/Y/H/G, -/S/Y/G/D, S/Y/G/D, S/Y/G/T, P/L, Y/S/H/L/F, T”. A preferred example a light chain CDR3 sequence within this definition consists of the sequence “Q, Q, S, Y, S, T, P, Y, T”

Binding domain B1 may comprise at least a heavy chain CDR3 as defined in (c) and/or a light chain CDR3 as defined in (f). Binding domain B1 may comprise all three heavy chain CDR sequences of (a), (b) and (c) and/or all three light chain CDR sequences of (d), (e) and (f).

Exemplary CDR sequences are recited in Tables C(1) and C(2), SEQ ID NOs: 32 to 66, and SEQ ID NOs 26 to 27.

Preferred OX40 binding domains may comprise at least a heavy chain CDR3 as defined in any individual row of Table C(1) and/or a light chain CDR3 as defined in in any individual row of Table C(2). Binding domain B1 may comprise all three heavy chain CDR sequences shown in an individual row of Table C(1) (that is, all three heavy chain CDRs of a given “VH number”) and/or all three light chain CDR sequences shown in an individual row of Table C(2) (that is, all three light chain CDRs of a given “VL number”).

Examples of complete heavy and light chain variable region amino acid sequences are shown in Table D. Exemplary nucleic acid sequences encoding each amino acid sequence are also shown. The numbering of said VH and VL regions in Table D corresponds to the numbering system used as in Table C(1) and C(2). Thus, for example, the amino acid sequence for “1167, light chain VL” is an example of a complete VL region sequence comprising all three CDRs of VL number 1167 shown in Table C(2) and the amino acid sequence for “1166, heavy chain VH” is an example of a complete VH region sequence comprising all three CDRs of VH number 1166 shown in Table C(1).

In exemplary embodiments, binding domain B1 comprises:
(a) the three CDRs of the heavy chain and/or the three CDRs of the light chain of antibody 1166/1167 (SEQ ID NOs: 32, 40 and 49 and/or SEQ ID NOs: 26, 27 and 60);
(b) the three CDRs of the heavy chain and/or the three CDRs of the light chain of antibody 1170/1171 (SEQ ID NOs: 32, 41 and 50 and/or SEQ ID NOs: 26, 27 and 61);
(c) the three CDRs of the heavy chain and/or the three CDRs of the light chain of antibody 1164/1135 (SEQ ID NOs: 33, 42 and 51 and/or SEQ ID NOs: 26, 27 and 62);
(d) the three CDRs of the heavy chain and/or the three CDRs of the light chain of antibody 1168/1135 (SEQ ID NOs: 34, 43 and 52 and/or SEQ ID NOs: 26, 27 and 62);
(e) the three CDRs of the heavy chain and/or the three CDRs of the light chain of antibody 1482/1483 (SEQ ID NOs: 35, 44 and 53 and/or SEQ ID NOs: 26, 27 and 63);
(f) the three CDRs of the heavy chain and/or the three CDRs of the light chain of antibody 1490/1135 (SEQ ID NOs: 35, 43 and 54 and/or SEQ ID NOs: 26, 27 and 62);
(g) the three CDRs of the heavy chain and/or the three CDRs of the light chain of antibody 1514/1515 (SEQ ID NOs: 36, 45 and 55 and/or SEQ ID NOs: 26, 27 and 64);
(h) the three CDRs of the heavy chain and/or the three CDRs of the light chain of antibody 1520/1135 (SEQ ID NOs 35, 40 and 56 and/or SEQ ID NOs: 26, 27 and 62);
(i) the three CDRs of the heavy chain and/or the three CDRs of the light chain of antibody 1524/1525 (SEQ ID NOs: 37, 46 and 57 and/or SEQ ID NOs: 26, 27 and 65);
(j) the three CDRs of the heavy chain and/or the three CDRs of the light chain of antibody 1526/1527 (SEQ ID NOs: 38, 47 and 58 and/or SEQ ID NOs: 26, 27 and 66);
(k) the three CDRs of the heavy chain and/or the three CDRs of the light chain of antibody 1542/1135 (SEQ ID NOs: 39, 48 and 59 and/or SEQ ID NOs: 26, 27 and 62); or
(l) the three CDRs of the heavy chain and/or the three CDRs of the light chain of antibody 1170/1167 (SEQ ID NOs: 32, 41 and 50 and/or SEQ ID NOs: 26, 27 and 60).

wherein the numbering of the antibody (e.g. Antibody X/Y) defines the heavy chain variable region (X) and the light chain variable region (Y), respectively (or, where a single number is indicated, the heavy chain variable region [X] only is defined).

Thus, binding domain B1 may comprise:
(a) the heavy chain variable region and/or the light chain variable region of antibody 1166/1167 (SEQ ID NO: 69 and/or SEQ ID NO: 67);
(b) the heavy chain variable region and/or the light chain variable region of antibody 1170/1171 (SEQ ID NO: 73 and/or SEQ ID NO: 71);
(c) the heavy chain variable region and/or the light chain variable region of antibody 1164/1135 (SEQ ID NO: 77 and/or SEQ ID NO: 75);
(d) the heavy chain variable region and/or the light chain variable region of antibody 1168/1135 (SEQ ID NO: 79 and/or SEQ ID NO: 75);
(e) the heavy chain variable region and/or the light chain variable region of antibody 1482/1483 (SEQ ID NO: 83 and/or SEQ ID NO: 81);
(f) the heavy chain variable region and/or the light chain variable region of antibody 1490/1135 (SEQ ID NO: 85 and/or SEQ ID NO: 75);
(g) the heavy chain variable region and/or the light chain variable region of antibody 1514/1515 (SEQ ID NO: 89 and/or SEQ ID NO: 87);
(h) the heavy chain variable region and/or the light chain variable region of antibody 1520/1135 (SEQ ID NO: 91 and/or SEQ ID NO: 75);
(i) the heavy chain variable region and/or the light chain variable region of antibody 1524/1525 (SEQ ID NO: 95 and/or SEQ ID NO: 93);
(j) the heavy chain variable region and/or the light chain variable region of antibody 1526/1527 (SEQ ID NO: 99 and/or SEQ ID NO: 97);
(k) the heavy chain variable region and/or the light chain variable region of antibody 1542/1135 (SEQ ID NO: 101 and/or SEQ ID NO: 75); or
(l) the heavy chain variable region and/or the light chain variable region of antibody 1170/1167 (SEQ ID NO: 73 and/or SEQ ID NO: 67).

It will be appreciated by persons skilled in the art that the bispecific polypeptides of the invention may alternatively comprise variants of the above-defined variable regions.

A variant of any one of the heavy or light chain amino acid sequences recited herein may be a substitution, deletion or addition variant of said sequence. A variant may comprise 1, 2, 3, 4, 5, up to 10, up to 20, up to 30 or more amino acid substitutions and/or deletions from the said sequence. “Deletion” variants may comprise the deletion of individual amino acids, deletion of small groups of amino acids such as 2, 3, 4 or 5 amino acids, or deletion of larger amino acid regions, such as the deletion of specific amino acid domains or other features. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows:

| | |
|---|---|
| Ala, A | aliphatic, hydrophobic, neutral |
| Cys, C | polar, hydrophobic, neutral |
| Asp, D | polar, hydrophilic, charged (−) |
| Glu, E | polar, hydrophilic, charged (−) |
| Phe, F | aromatic, hydrophobic, neutral |
| Gly, G | aliphatic, neutral |
| His, H | aromatic, polar, hydrophilic, charged (+) |
| Ile, I | aliphatic, hydrophobic, neutral |
| Lys, K | polar, hydrophilic, charged(+) |
| Leu, L | aliphatic, hydrophobic, neutral |
| Met, M | hydrophobic, neutral |
| Asn, N | polar, hydrophilic, neutral |
| Pro, P | hydrophobic, neutral |
| Gln, Q | polar, hydrophilic, neutral |
| Arg, R | polar, hydrophilic, charged (+) |
| Ser, S | polar, hydrophilic, neutral |
| Thr, T | polar, hydrophilic, neutral |
| Val, V | aliphatic, hydrophobic, neutral |
| Trp, W | aromatic, hydrophobic, neutral |
| Tyr, Y | aromatic, polar, hydrophobic |

Amino acids herein may be referred to by full name, three letter code or single letter code.

Preferred “derivatives” or “variants” include those in which instead of the naturally occurring amino acid the amino acid which appears in the sequence is a structural analog thereof. Amino acids used in the sequences may also be derivatised or modified, e.g. labelled, providing the function of the antibody is not significantly adversely affected.

Derivatives and variants as described above may be prepared during synthesis of the antibody or by post-production modification, or when the antibody is in recombinant form using the known techniques of site-directed mutagenesis, random mutagenesis, or enzymatic cleavage and/or ligation of nucleic acids.

Preferably variants have an amino acid sequence which has more than 60%, or more than 70%, e.g. 75 or 80%, preferably more than 85%, e.g. more than 90 or 95% amino acid identity to a sequence as shown in the sequences disclosed herein. This level of amino acid identity may be seen across the full length of the relevant SEQ ID NO sequence or over a part of the sequence, such as across 20, 30, 50, 75, 100, 150, 200 or more amino acids, depending on the size of the full length polypeptide.

In connection with amino acid sequences, “sequence identity” refers to sequences which have the stated value when assessed using ClustalW (Thompson et al., 1994; the disclosures of which are incorporated herein by reference) with the following parameters:

Pairwise alignment parameters—Method: accurate, Matrix: PAM, Gap open penalty: 10.00, Gap extension penalty: 0.10;

Multiple alignment parameters—Matrix: PAM, Gap open penalty: 10.00, % identity for delay: 30, Penalize end gaps: on, Gap separation distance: 0, Negative matrix: no, Gap extension penalty: 0.20, Residue-specific gap penalties: on, Hydrophilic gap penalties: on, Hydrophilic residues: GPSNDQEKR. Sequence identity at a particular residue is intended to include identical residues which have simply been derivatised.

Thus, in one embodiment binding domain B1 may comprises one or more variants of the above-defined light chain variable regions and/or said heavy chain variable regions having at least 90% sequence identity thereto.

In preferred embodiments, binding domain B1 comprises:
(a) the light chain and/or the heavy chain of antibody 1166/1167;
(b) the light chain and/or the heavy chain of antibody 1170/1171;
(c) the light chain and/or the heavy chain of antibody 1164/1135;
(d) the light chain and/or the heavy chain of antibody 1168/1135;
(e) the light chain and/or the heavy chain of antibody 1482/1483;
(f) the light chain and/or the heavy chain of antibody 1490/1135;
(g) the light chain and/or the heavy chain of antibody 1514/1515;
(h) the light chain and/or the heavy chain of antibody 1520/1135;
(i) the light chain and/or the heavy chain of antibody 1524/1525;
(j) the light chain and/or the heavy chain of antibody 1526/1527;
(k) the light chain and/or the heavy chain of antibody 1542/1135; or
(l) the light chain and/or the heavy chain of antibody 1170/1167.

In exemplary embodiments, binding domain B1 comprises:
(a) the three CDRs of the heavy chain and/or the three CDRs of the light chain of antibody 1170/1171 (SEQ ID NOs: 32, 41 and 50 and/or SEQ ID NOs: 26, 27 and 61), or the exemplary heavy and light chain variable regions, or heavy and light antibody chains, which comprise said CDRs, as detailed above; or
(b) the three CDRs of the heavy chain and/or the three CDRs of the light chain of antibody 1526/1527 (SEQ ID NOs: 38, 47 and 58 and/or SEQ ID NOs: 26, 27 and 66), or the exemplary heavy and light chain variable regions, or heavy and light antibody chains, which comprise said CDRs, as detailed above; or
(c) the three CDRs of the heavy chain and/or the three CDRs of the light chain of antibody 1168/1135 (SEQ ID NOs: 34, 43 and 52 and/or SEQ ID NOs: 26, 27 and 62); or the exemplary heavy and light chain variable regions, or heavy and light antibody chains, which comprise said CDRs, as detailed above.

Tumour Cell-Targeting Domains

The bispecific polypeptides of the invention further comprise a binding domain (B2) which is capable of specifically binding a tumour cell-associated antigen.

By “tumour cell-associated antigen” we include proteins accessible on the extracellular surface of tumour cells, such that they are accessible to the bispecific polypeptides of the invention following administration into the body. In one embodiment, the tumour cell-associated antigen is tumour specific, i.e. it is found exclusively on tumour cells and not on normal, healthy cells. However, it will be appreciated by persons skilled in the art that the tumour cell-associated antigen may be preferentially expressed on tumour cells, i.e. it is expressed on tumour cells at a higher level than on normal, healthy cells (thus, expression of the antigen on tumour cells may be at least five times more than on normal, healthy cells, for example expression levels on tumour cells of at least ten times more, twenty times more, fifty time more or greater).

In one embodiment, binding domain B2 binds to a tumour cell-associated antigen selected from the group consisting of:
(a) products of mutated oncogenes and tumour suppressor genes;
(b) overexpressed or aberrantly expressed cellular proteins;
(c) tumour antigens produced by oncogenic viruses;
(d) oncofetal antigens;
(e) altered cell surface glycolipids and glycoproteins;
(f) cell type-specific differentiation antigens;
(g) hypoxia-induced antigens;
(h) tumour peptides presented by MHC class 1;
(i) epithelial tumour antigens;
(j) haematological tumour-associated antigens;
(k) cancer testis antigens; and
(l) melanoma antigens.

Thus, the tumour cell-associated antigen may be selected from the group consisting of 5T4, CD20, CD19, MUC-1, carcinoembryonic antigen (CEA), CA-125, C017-1A, EpCAM, HER2, EphA2, EphA3, DR5, FAP, OGD2, VEGFR, Her3 and EGFR.

In one embodiment, the tumour cell-associated antigen is selected from the group consisting of 5T4, EpCAM, HER2 and EGFR.

In one embodiment, the tumour cell-associated antigen is an oncofetal antigen. For example, the tumour cell-associated antigen may be 5T4.

In one embodiment, the tumour cell is a solid tumour cell.

For example, the solid tumour may be selected from the groups consisting of renal cell carcinoma, colorectal cancer, lung cancer, prostate cancer, breast cancer, melanomas, bladder cancer, brain/CNS cancer, cervical cancer, oesophageal cancer, gastric cancer, head/neck cancer, kidney cancer, liver cancer, lymphomas, ovarian cancer, pancreatic cancer and sarcomas.

Advantageously, binding domain B2 binds to the tumour cell-associated antigen with a K_{D} of less than 10×10⁻⁹M, for example less than 4×10⁻⁹M or less than 1.2×10⁻⁹M.

In exemplary embodiments, binding domain B2 comprises:
(a) the three CDRs of the light chain and/or the three CDRs of the heavy chain of antibody 1206/1207 (SEQ ID NOs: 26, 27 and 28 and/or SEQ ID NOs: 17, 19 and 22);
(b) the three CDRs of the light chain and/or the three CDRs of the heavy chain of antibody 1208/1135 (SEQ ID NOs: 26, 27 and 29 and/or SEQ ID NOs: 18, 20 and 23);
(c) the three CDRs of the light chain and/or the three CDRs of the heavy chain of antibody 1210/1211 (SEQ ID NOs: 26, 27 and 30 and/or SEQ ID NOs: 18, 20 and 24); or
(d) the three CDRs of the light chain and/or the three CDRs of the heavy chain of antibody 1212/1213 (SEQ ID NOs: 26, 27 and 31 and/or SEQ ID NOs: 18, 21 and 25).

In alternative embodiments, B2 can comprise CDRs selected from known antibodies to tumour associated antigens. For example, B2 may comprise the CDRs of an antibody to EpCAM, such as Edrecolomab (as disclosed in U.S. Pat. No. 7,557,190, the disclosure of which is incorporated herein by reference). Alternatively, B2 may comprise the CDRs of an antibody to EGFR, such as Cetuximab (as disclosed in U.S. Pat. No. 7,060,808, the disclosure of which is incorporated herein by reference). In a further embodiment, B2 may comprise the CDRs of an antibody to HER2, such as Herceptin (Drug Bank, Accession number: DB00072 (HER2), the disclosure of which is incorporated herein by reference).

Thus, binding domain B2 may comprise:
(a) the light chain variable region and/or the heavy chain variable region of antibody 1206/1207 (SEQ ID NO: 3 and/or SEQ ID NO: 1);
(b) the light chain variable region and/or the heavy chain variable region of antibody 1208/1135 (SEQ ID NO: 7 and/or SEQ ID NO: 5);
(c) the light chain variable region and/or the heavy chain variable region of antibody 1210/1211 (SEQ ID NO: 11 and/or SEQ ID NO: 9); or
(d) the light chain variable region and/or the heavy chain variable region of antibody 1212/1213 (SEQ ID NO: 15 and/or SEQ ID NO: 13).

Alternatively, B2 can comprise the heavy chain variable regions and/or light chain variable regions selected from known antibodies to tumour associated antigens, for example antibodies to EpCAM, EGFR and HER2, as described above.

It will be appreciated by skilled persons that binding domain B2 may alternatively comprise variants of said light chain variable regions and/or said heavy chain variable regions, for example having at least 90% sequence identity thereto.

In one embodiment, binding domain B2 comprises:
(a) the light chain and/or the heavy chain of antibody 1206/1207;
(b) the light chain and/or the heavy chain of antibody 1208/1135;
(c) the light chain and/or the heavy chain of antibody 1210/1211; or
(d) the light chain and/or the heavy chain of antibody 1212/1213.

Alternatively, B2 can comprise the heavy chain and/or light chain selected from known antibodies to tumour associated antigens, for example antibodies to EpCAM, EGFR and HER2, as described above.

Exemplary OX40-Tumour Cell-Associated Antigen Bispecific Antibodies

In one preferred embodiment of the bispecific polypeptides of the invention, binding domain B1 is an IgG and binding domain B2 is an scFv. Conversely, binding domain B1 may be an scFv and binding domain B2 may be an IgG.

Bispecific polypeptides of the invention may comprise the CDRs of the light chains of any of the B1 domains described above, and/or the CDRs of the heavy chains of any of the B1 domains described above, in combination with any of the CDRs of the light chains of any of the B2 domains described above, and/or the CDRs of the heavy chains of any of the B2 domains described above.

For example, in one embodiment of the invention B2 comprises the 3 CDRs of the light chain of antibody 1206/1207 and/or the 3 CDRs of the heavy chain of antibody 1206/1207 (SEQ ID NOs: 17, 19 and 22 and/or SEQ ID NOs 26, 27 and 28) or the corresponding heavy chain variable region and/or light chain variable region (SEQ ID NO: 1 and SEQ ID NO: 3); and B1 comprises the heavy chain CDR sequences of an antibody selected from Table C(1) and/or the light chain CDR sequences of an antibody selected from Table C(2) or the corresponding heavy chain variable region and/or light chain variable region, as laid out in Table D.

In a further embodiment of the invention B2 comprises the 3 CDRs of the light chain of antibody 1208/1135 and/or the 3 CDRs of the heavy chain of antibody 1208/1135 (SEQ ID NOs: 18, 20 and 23 and/or SEQ ID NOs 26, 27 and 29) or the corresponding heavy chain variable region and/or light chain variable region (SEQ ID NO: 5 and SEQ ID NO: 7); and B1 comprises the heavy chain CDR sequences of an antibody selected from Table C(1) and/or the light chain CDR sequences of an antibody selected from Table C(2) or the corresponding heavy chain variable region and/or light chain variable region, as laid out in Table D.

In a further embodiment of the invention B2 comprises the 3 CDRs of the light chain of antibody 1210/1211 and/or the 3 CDRs of the heavy chain of antibody 1210/1211 (SEQ ID NOs: 18, 20 and 24 and/or SEQ ID NOs 26, 27 and 30) or the corresponding heavy chain variable region and/or light chain variable region (SEQ ID NO: 9 and SEQ ID NO: 11); and B1 comprises the heavy chain CDR sequences of an antibody selected from Table C(1) and/or the light chain CDR sequences of an antibody selected from Table C(2) or the corresponding heavy chain variable region and/or light chain variable region, as laid out in Table D.

In a further embodiment of the invention B2 comprises the 3 CDRs of the light chain of antibody 1212/1213 and/or the 3 CDRs of the heavy chain of antibody 1212/1213 (SEQ ID NOs: 18, 21 and 25 and/or SEQ ID NOs 26, 27 and 31) or the corresponding heavy chain variable region and/or light chain variable region (SEQ ID NO: 13 and SEQ ID NO: 15); and B1 comprises the heavy chain CDR sequences of an antibody selected from Table C(1) and/or the light chain CDR sequences of an antibody selected from Table C(2) or the corresponding heavy chain variable region and/or light chain variable region, as laid out in Table D.

In a further embodiment of the invention B2 comprises the 3 CDRs of the light chain of a commercially available antibody to EpCAM, as described above, and/or the 3 CDRs of the light chain of the same antibody, or the corresponding heavy chain variable region and/or light chain variable region; and B1 comprises the heavy chain CDR sequences of an antibody selected from Table C(1) and/or the light chain CDR sequences of an antibody selected from Table C(2) or the corresponding heavy chain variable region and/or light chain variable region, as laid out in Table D.

In a further embodiment of the invention B2 comprises the 3 CDRs of the light chain of a commercially available antibody to EGFR, as described above, and/or the 3 CDRs of the light chain of the same antibody, or the corresponding heavy chain variable region and/or light chain variable region; and B1 comprises the heavy chain CDR sequences of an antibody selected from Table C(1) and/or the light chain CDR sequences of an antibody selected from Table C(2) or the corresponding heavy chain variable region and/or light chain variable region, as laid out in Table D.

In a further embodiment of the invention B2 comprises the 3 CDRs of the light chain of a commercially available antibody to HER2, as described above, and/or the 3 CDRs of the light chain of the same antibody, or the corresponding heavy chain variable region and/or light chain variable region; and B1 comprises the heavy chain CDR sequences of an antibody selected from Table C(1) and/or the light chain CDR sequences of an antibody selected from Table C(2) or the corresponding heavy chain variable region and/or light chain variable region, as laid out in Table D.

Thus, in exemplary bispecific polypeptides of the invention:
(a) B1 comprises the three CDRs of the heavy chain and/or the three CDRs of the light chain of antibody 1170/1167 (SEQ ID NOs: 32, 41 and 50 and/or SEQ ID NOs: 26, 27 and 60) and B2 comprises the three CDRs of the heavy chain and/or the three CDRs of the light chain of antibody 1210/1211 (SEQ ID NOs: 18, 20 and 24 and/or SEQ ID NOs: 26, 27 and 30);
(b) B1 comprises the three CDRs of the heavy chain and/or the three CDRs of the light chain of antibody 1482/1483 (SEQ ID NOs: 35, 44 and 53 and/or SEQ ID NOs: 26, 27 and 63) and B2 comprises the three CDRs of the heavy chain and/or the three CDRs of the light chain of antibody 1210/1211 (SEQ ID NOs: 18, 20 and 24 and/or SEQ ID NOs: 26, 27 and 30);
(c) B1 comprises the three CDRs of the heavy chain and/or the three CDRs of the light chain of antibody 1170/1167 (SEQ ID NOs: 32, 41 and 50 and/or SEQ ID NOs: 26, 27 and 60) and B2 comprises the three CDRs of the heavy chain and/or the three CDRs of the light chain of antibody 1208/1135 (SEQ ID NOs: 18, 20 and 23 and/or SEQ ID NOs: 26, 27 and 29);
(d) B1 comprises the three CDRs of the heavy chain and/or the three CDRs of the light chain of antibody 1482/1483 (SEQ ID NOs: 35, 44 and 53 and/or SEQ ID NOs: 26, 27 and 63) and B2 comprises the three CDRs of the heavy chain and/or the three CDRs of the light chain of antibody 1208/1135 (SEQ ID NOs: 18, 20 and 23 and/or SEQ ID NOs: 26, 27 and 29);
(e) B1 comprises the three CDRs of the heavy chain and/or the three CDRs of the light chain of antibody 1166/1167 (SEQ ID NOs: 32, 40 and 49 and/or SEQ ID NOs: 26, 27 and 60) and B2 comprises the three CDRs of the heavy chain and/or the three CDRs of the light chain of antibody 1210/1211 (SEQ ID NOs: 18, 20 and 24 and/or SEQ ID NOs: 26, 27 and 30);
(f) B1 comprises the three CDRs of the heavy chain and/or the three CDRs of the light chain of antibody 1170/1171 (SEQ ID NOs: 32, 41 and 50 and/or SEQ ID NOs: 26, 27 and 61) and B2 comprises the three CDRs of the heavy chain and/or the three CDRs of the light chain of antibody 1210/1211 (SEQ ID NOs: 18, 20 and 24 and/or SEQ ID NOs: 26, 27 and 30);
(g) B1 comprises the three CDRs of the heavy chain and/or the three CDRs of the light chain of antibody 1166/1167 (SEQ ID NOs: 32, 40 and 49 and/or SEQ ID NOs: 26, 27 and 60) and B2 comprises the three CDRs of the heavy chain and/or the three CDRs of the light chain of antibody 1208/1135 (SEQ ID NOs: 18, 20 and 23 and/or SEQ ID NOs: 26, 27 and 29);
(h) B1 comprises the three CDRs of the heavy chain and/or the three CDRs of the light chain of antibody 1170/1171 (SEQ ID NOs: 32, 41 and 50 and/or SEQ ID NOs: 26, 27 and 61) and B2 comprises the three CDRs of the heavy chain and/or the three CDRs of the light chain of antibody 1208/1135 (SEQ ID NOs: 18, 20 and 23 and/or SEQ ID NOs: 26, 27 and 29);
(i) B1 comprises the three CDRs of the heavy chain and/or the three CDRs of the light chain of antibody 1526/1527 (SEQ ID NOs: 38, 47 and 58 and/or SEQ ID NOs: 26, 27 and 66) and B2 comprises the three CDRs of the heavy chain and/or the three CDRs of the light chain of antibody 1210/1211 (SEQ ID NOs: 18, 20 and 24 and/or SEQ ID NOs: 26, 27 and 30); or
(j) B1 comprises the three CDRs of the heavy chain and/or the three CDRs of the light chain of antibody 1526/1527 (SEQ ID NOs: 38, 47 and 58 and/or SEQ ID NOs: 26, 27 and 66) and B2 comprises the three CDRs of the heavy chain and/or the three CDRs of the light chain of antibody 1208/1135 (SEQ ID NOs: 18, 20 and 23 and/or SEQ ID NOs: 26, 27 and 29).

Thus, in certain embodiments:
(a) B1 comprises the heavy chain variable region and/or the light chain variable region of antibody 1170/1167 (SEQ ID NO: 73 and/or SEQ ID NO: 67) and B2 comprises the heavy chain variable region and/or the light chain variable region of antibody 1210/1211 (SEQ ID NO: 9 and/or SEQ ID NO: 11);
(b) B1 comprises the heavy chain variable region and/or the light chain variable region of antibody 1482/1483 (SEQ ID NO: 83 and/or SEQ ID NO: 81) and B2 comprises the heavy chain variable region and/or the light chain variable region of antibody 1210/1211 (SEQ ID NO: 9 and/or SEQ ID NO: 11);
(c) B1 comprises the heavy chain variable region and/or the light chain variable region of antibody 1170/1167 (SEQ ID NO: 73 and/or SEQ ID NO: 67) and B2 comprises the heavy chain variable region and/or the light chain variable region of antibody 1208/1135 (SEQ ID NO: 5 and/or SEQ ID NO: 7);
(d) 81 comprises the heavy chain variable region and/or the light chain variable region of antibody 1482/1483 (SEQ ID NO: 83 and/or SEQ ID NO: 81) and B2 comprises the heavy chain variable region and/or the light chain variable region of antibody 1208/1135 (SEQ ID NO: 5 and/or SEQ ID NO: 7);
(e) B1 comprises the heavy chain variable region and/or the light chain variable region of antibody 1166/1167 (SEQ ID NO: 69 and/or SEQ ID NO: 67) and B2 comprises the heavy chain variable region and/or the light chain variable region of antibody 1210/1211 (SEQ ID NO: 9 and/or SEQ ID NO: 11);
(f) B1 comprises the heavy chain variable region and/or the light chain variable region of antibody 1170/1171 (SEQ ID NO: 73 and/or SEQ ID NO: 71) and B2 comprises the heavy chain variable region and/or the light chain variable region of antibody 1210/1211 (SEQ ID NO: 9 and/or SEQ ID NO: 11);
(g) B1 comprises the heavy chain variable region and/or the light chain variable region of antibody 1166/1167 (SEQ ID NO: 69 and/or SEQ ID NO: 67) and B2 comprises the heavy chain variable region and/or the light chain variable region of antibody 1208/1135 (SEQ ID NO: 5 and/or SEQ ID NO: 7);
(h) B1 comprises the heavy chain variable region and/or the light chain variable region of antibody 1170/1171 (SEQ ID NO: 73 and/or SEQ ID NO: 71) and B2 comprises the heavy chain variable region and/or the light chain variable region of antibody 1208/1135 (SEQ ID NO: 5 and/or SEQ ID NO: 7);
(i) B1 comprises the heavy chain variable region and/or the light chain variable region of antibody 1526/1527 (SEQ ID NO: 99 and/or SEQ ID NO: 97); and B2 comprises the heavy chain variable region and/or the light chain variable region of antibody 1210/1211 (SEQ ID NO: 9 and/or SEQ ID NO: 11);
(j) B1 comprises the heavy chain variable region and/or the light chain variable region of antibody 1526/1527 (SEQ ID NO: 99 and/or SEQ ID NO: 97); and B2 comprises the heavy chain variable region and/or the light chain variable region of antibody 1208/1135 (SEQ ID NO: 5 and/or SEQ ID NO: 7); or
(k) variants of said light chain variable regions and/or said heavy chain variable regions, for example having at least 90% sequence identity thereto (as discussed above).

The B1 domain may comprise the light chain variable region and/or the heavy chain variable region of any B1 domain described above, and the B2 domain may comprise the light chain variable region and/or the heavy chain variable region of any B2 domain described above, or variants of said light chain variable regions and/or said heavy chain variable regions having at least 90% sequence identity thereto.

Thus, preferred bispecific polypeptides of the invention include:
(a) B1 comprises the three CDRs of the heavy chain and/or the three CDRs of the light chain of antibody 1170/1171 (SEQ ID NOs: 32, 41 and 50 and/or SEQ ID NOs: 26, 27 and 61), or variable regions or antibody chains comprising said CDRs, as detailed above, and B2 comprises the three CDRs of the heavy chain and/or the three CDRs of the light chain of antibody 1210/1211 (SEQ ID NOs: 18, 20 and 24 and/or SEQ ID NOs: 26, 27 and 30) or variable regions or antibody chains comprising said CDRs, as detailed above;
(b) B1 comprises the three CDRs of the heavy chain and/or the three CDRs of the light chain of antibody 1170/1171 (SEQ ID NOs: 32, 41 and 50 and/or SEQ ID NOs: 26, 27 and 61) or variable regions or antibody chains comprising said CDRs, as detailed above, and B2 comprises the three CDRs of the heavy chain and/or the three CDRs of the light chain of antibody 1208/1135 (SEQ ID NOs: 18, 20 and 23 and/or SEQ ID NOs: 26, 27 and 29) or variable regions or antibody chains comprising said CDRs, as detailed above;
(c) B1 comprises the three CDRs of the heavy chain and/or the three CDRs of the light chain of antibody 1526/1527 (SEQ ID NOs: 38, 47 and 58 and/or SEQ ID NOs: 26, 27 and 66) or variable regions or antibody chains comprising said CDRs, as detailed above, and B2 comprises the three CDRs of the heavy chain and/or the three CDRs of the light chain of antibody 1210/1211 (SEQ ID NOs: 18, 20 and 24 and/or SEQ ID NOs: 26, 27 and 30) or variable regions or antibody chains comprising said CDRs, as detailed above; or
(d) B1 comprises the three CDRs of the heavy chain and/or the three CDRs of the light chain of antibody 1526/1527 (SEQ ID NOs: 38, 47 and 58 and/or SEQ ID NOs: 26, 27 and 66) or variable regions or antibody chains comprising said CDRs, as detailed above, and B2 comprises the three CDRs of the heavy chain and/or the three CDRs of the light chain of antibody 1208/1135 (SEQ ID NOs: 18, 20 and 23 and/or SEQ ID NOs: 26, 27 and 29) or variable regions or antibody chains comprising said CDRs, as detailed above.

In one particularly preferred embodiment, B1 comprises the three CDRs of the heavy chain and/or the three CDRs of the light chain of antibody 1170/1171 (SEQ ID NOs: 32, 41 and 50 and/or SEQ ID NOs: 26, 27 and 61), or variable regions or antibody chains comprising said CDRs, as detailed above, and B2 comprises the three CDRs of the heavy chain and/or the three CDRs of the light chain of antibody 1210/1211 (SEQ ID NOs: 18, 20 and 24 and/or SEQ ID NOs: 26, 27 and 30) or variable regions or antibody chains comprising said CDRs, as detailed above.

Typically, the bispecific antibody polypeptides of the invention will comprise constant region sequences, in addition to the above-defined variable region sequences.

An exemplary heavy chain constant region amino acid sequence which may be combined with any VH region sequence disclosed herein (to form a complete heavy chain) is the following IgG1 heavy chain constant region sequence:

[SEQ ID NO: 111]
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

Likewise, an exemplary light chain constant region amino acid sequence which may be combined with any VL region sequence disclosed herein (to form a complete light chain) is the kappa chain constant region sequence reproduced here:

[SEQ ID NO: 112]
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

Other light chain constant region sequences are known in the art and could also be combined with any VL region disclosed herein.

As discussed above, methods for the production of antibody polypeptides of the invention are well known in the art.

Conveniently, the antibody polypeptide is or comprises a recombinant polypeptide. Suitable methods for the production of such recombinant polypeptides are well known in the art, such as expression in prokaryotic or eukaryotic hosts cells (for example, see Green & Sambrook, 2012, Molecular Cloning, A Laboratory Manual, Fourth Edition, Cold Spring Harbor, N.Y., the relevant disclosures in which document are hereby incorporated by reference).

Antibody polypeptides of the invention can also be produced using a commercially available in vitro translation system, such as rabbit reticulocyte lysate or wheatgerm lysate (available from Promega). Preferably, the translation system is rabbit reticulocyte lysate. Conveniently, the translation system may be coupled to a transcription system, such as the TNT transcription-translation system (Promega). This system has the advantage of producing suitable mRNA transcript from an encoding DNA polynucleotide in the same reaction as the translation.

It will be appreciated by persons skilled in the art that antibody polypeptides of the invention may alternatively be synthesised artificially, for example using well known liquid-phase or solid phase synthesis techniques (such as t-Boc or Fmoc solid-phase peptide synthesis).

Polynucleotides, Vectors and Cells

A second aspect of the invention provides an isolated nucleic acid molecule encoding a bispecific polypeptide according to any one of the preceding claims, or a component polypeptide chain thereof. For example, the nucleic acid molecule may comprise any of the nucleotide sequences provided in Tables A and D.

Thus, a polynucleotide of the invention may encode any polypeptide as described herein, or all or part of B1 or all or part of B2. The terms “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or substantially isolated form. By substantially isolated, it is meant that there may be substantial, but not total, isolation of the polypeptide from any surrounding medium. The polynucleotides may be mixed with carriers or diluents which will not interfere with their intended use and still be regarded as substantially isolated.

A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Representative polynucleotides which encode examples of a heavy chain or light chain amino acid sequence of an antibody may comprise or consist of any one of the nucleotide sequences disclosed herein, for example the sequences set out in Tables A and D.

A suitable polynucleotide sequence may alternatively be a variant of one of these specific polynucleotide sequences. For example, a variant may be a substitution, deletion or addition variant of any of the above nucleic acid sequences. A variant polynucleotide may comprise 1, 2, 3, 4, 5, up to 10, up to 20, up to 30, up to 40, up to 50, up to 75 or more nucleic acid substitutions and/or deletions from the sequences given in the sequence listing.

Suitable variants may be at least 70% homologous to a polynucleotide of any one of nucleic acid sequences disclosed herein, preferably at least 80 or 90% and more preferably at least 95%, 97% or 99% homologous thereto. Preferably homology and identity at these levels is present at least with respect to the coding regions of the polynucleotides. Methods of measuring homology are well known in the art and it will be understood by those of skill in the art that in the present context, homology is calculated on the basis of nucleic acid identity. Such homology may exist over a region of at least 15, preferably at least 30, for instance at least 40, 60, 100, 200 or more contiguous nucleotides. Such homology may exist over the entire length of the unmodified polynucleotide sequence.

Methods of measuring polynucleotide homology or identity are known in the art. For example the UWGCG Package provides the BESTFIT program which can be used to calculate homology (e.g. used on its default settings) (Devereux et al, 1984; the disclosures of which are incorporated herein by reference).

The PILEUP and BLAST algorithms can also be used to calculate homology or line up sequences (typically on their default settings), for example as described in Altschul, 1993; Altschul et al, 1990, the disclosures of which are incorporated herein by reference).

Software for performing BLAST analysis is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff & Henikoff, 1992; the disclosures of which are incorporated herein by reference) alignments (B) of 50, expectation (E) of 10, M=5, N=4, and a comparison of both strands.

The BLAST algorithm performs a statistical analysis of the similarity between two sequences; see e.g. Karlin & Altschul, 1993; the disclosures of which are incorporated herein by reference. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologue may differ from a sequence in the relevant polynucleotide by less than 3, 5, 10, 15, 20 or more mutations (each of which may be a substitution, deletion or insertion). These mutations may be measured over a region of at least 30, for instance at least 40, 60 or 100 or more contiguous nucleotides of the homologue.

In one embodiment, a variant sequence may vary from the specific sequences given in the sequence listing by virtue of the redundancy in the genetic code. The DNA code has 4 primary nucleic acid residues (A, T, C and G) and uses these to “spell” three letter codons which represent the amino acids the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons. A variant polynucleotide of the invention may therefore encode the same polypeptide sequence as another polynucleotide of the invention, but may have a different nucleic acid sequence due to the use of different codons to encode the same amino acids.

A polypeptide of the invention may thus be produced from or delivered in the form of a polynucleotide which encodes, and is capable of expressing, it.

Polynucleotides of the invention can be synthesised according to methods well known in the art, as described by way of example in Green & Sambrook (2012, Molecular Cloning—a laboratory manual, 4^{th} edition; Cold Spring Harbor Press; the disclosures of which are incorporated herein by reference).

The nucleic acid molecules of the present invention may be provided in the form of an expression cassette which includes control sequences operably linked to the inserted sequence, thus allowing for expression of the polypeptide of the invention in vivo. These expression cassettes, in turn, are typically provided within vectors (e.g., plasmids or recombinant viral vectors). Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art (see Green & Sambrook, supra).

The invention also includes cells that have been modified to express a polypeptide of the invention. Such cells include transient, or preferably stable higher eukaryotic cell lines, such as mammalian cells or insect cells, lower eukaryotic cells, such as yeast or prokaryotic cells such as bacterial cells. Particular examples of cells which may be modified by insertion of vectors or expression cassettes encoding for a polypeptide of the invention include mammalian HEK293T, CHO, HeLa, NSO and COS cells. Preferably the cell line selected will be one which is not only stable, but also allows for mature glycosylation and cell surface expression of a polypeptide.

Such cell lines of the invention may be cultured using routine methods to produce a polypeptide of the invention, or may be used therapeutically or prophylactically to deliver antibodies of the invention to a subject. Alternatively, polynucleotides, expression cassettes or vectors of the invention may be administered to a cell from a subject ex vivo and the cell then returned to the body of the subject.

In one embodiment, the nucleic acid molecule encodes an antibody heavy chain or variable region thereof.

In one embodiment, the nucleic acid molecule encodes an antibody light chain or variable region thereof.

By “nucleic acid molecule” we include DNA (e.g. genomic DNA or complementary DNA) and mRNA molecules, which may be single- or double-stranded. By “isolated” we mean that the nucleic acid molecule is not located or otherwise provided within a cell.

In one embodiment, the nucleic acid molecule is a cDNA molecule.

It will be appreciated by persons skilled in the art that the nucleic acid molecule may be codon-optimised for expression of the antibody polypeptide in a particular host cell, e.g. for expression in human cells (for example, see Angov, 2011, the disclosures of which are incorporated herein by reference).

Also included within the scope of the invention are the following:
(a) a third aspect of the invention provides a vector (such as an expression vector) comprising a nucleic acid molecule according to the second aspect of the invention;
(b) a fourth aspect of the invention provides a host cell (such as a mammalian cell, e.g. human cell, or Chinese hamster ovary cell, e.g. CHOK1SV cells) comprising a nucleic acid molecule according to the second aspect of the invention or a vector according to the third aspect of the invention; and
(c) a fifth aspect of the invention provides a method of making an antibody polypeptide according to the first aspect of the invention comprising culturing a population of host cells according to the fourth aspect of the invention under conditions in which said polypeptide is expressed, and isolating the polypeptide therefrom.

In a sixth aspect, the present invention provides compositions comprising molecules of the invention, such as the antibodies, bispecific polypeptides, polynucleotides, vectors and cells described herein. For example, the invention provides a composition comprising one or more molecules of the invention, such as one or more antibodies and/or bispecific polypeptides of the invention, and at least one pharmaceutically acceptable carrier.

It will be appreciated by persons skilled in the art that additional compounds may also be included in the pharmaceutical compositions, including, chelating agents such as EDTA, citrate, EGTA or glutathione.

The pharmaceutical compositions may be prepared in a manner known in the art that is sufficiently storage stable and suitable for administration to humans and animals. For example, the pharmaceutical compositions may be lyophilised, e.g. through freeze drying, spray drying, spray cooling, or through use of particle formation from supercritical particle formation.

By “pharmaceutically acceptable” we mean a non-toxic material that does not decrease the effectiveness of the OX40 and 5T4-binding activity of the antibody polypeptide of the invention. Such pharmaceutically acceptable buffers, carriers or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000), the disclosures of which are incorporated herein by reference).

The term “buffer” is intended to mean an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

The term “diluent” is intended to mean an aqueous or non-aqueous solution with the purpose of diluting the antibody polypeptide in the pharmaceutical preparation. The diluent may be one or more of saline, water, polyethylene glycol, propylene glycol, ethanol or oils (such as safflower oil, corn oil, peanut oil, cottonseed oil or sesame oil).

The term “adjuvant” is intended to mean any compound added to the formulation to increase the biological effect of the antibody polypeptide of the invention. The adjuvant may be one or more of zinc, copper or silver salts with different anions, for example, but not limited to fluoride, chloride, bromide, iodide, tiocyanate, sulfite, hydroxide, phosphate, carbonate, lactate, glycolate, citrate, borate, tartrate, and acetates of different acyl composition. The adjuvant may also be cationic polymers such as cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals. Examples of carbohydrates include lactose, glucose, sucrose, mannitol, and cyclodextrines, which are added to the composition, e.g. for facilitating lyophilisation. Examples of polymers are starch, cellulose ethers, cellulose carboxymethylcellulose, hydroxypropylmethyl cellulose, hydroxyethyl cellulose, ethylhydroxyethyl cellulose, alginates, carageenans, hyaluronic acid and derivatives thereof, polyacrylic acid, polysulphonate, polyethylenglycol/polyethylene oxide, polyethyleneoxide/polypropylene oxide copolymers, polyvinylalcohol/polyvinylacetate of different degree of hydrolysis, and polyvinylpyrrolidone, all of different molecular weight, which are added to the composition, e.g., for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The antibody polypeptides of the invention may be formulated into any type of pharmaceutical composition known in the art to be suitable for the delivery thereof.

In one embodiment, the pharmaceutical compositions of the invention may be in the form of a liposome, in which the antibody polypeptide is combined, in addition to other pharmaceutically acceptable carriers, with amphipathic agents such as lipids, which exist in aggregated forms as micelles, insoluble monolayers and liquid crystals. Suitable lipids for liposomal formulation include, without limitation, monoglycerides, diglycerides, sulfatides, lysolecithin, phospholipids, saponin, bile acids, and the like. Suitable lipids also include the lipids above modified by poly(ethylene glycol) in the polar headgroup for prolonging bloodstream circulation time. Preparation of such liposomal formulations is can be found in for example U.S. Pat. No. 4,235,871, the disclosures of which are incorporated herein by reference.

The pharmaceutical compositions of the invention may also be in the form of biodegradable microspheres. Aliphatic polyesters, such as poly(lactic acid) (PLA), poly(glycolic acid) (PGA), copolymers of PLA and PGA (PLGA) or poly(caprolactone) (PCL), and polyanhydrides have been widely used as biodegradable polymers in the production of microspheres. Preparations of such microspheres can be found in U.S. Pat. No. 5,851,451 and in EP 0 213 303, the disclosures of which are incorporated herein by reference.

In a further embodiment, the pharmaceutical compositions of the invention are provided in the form of polymer gels, where polymers such as starch, cellulose ethers, cellulose carboxymethylcellulose, hydroxypropylmethyl cellulose, hydroxyethyl cellulose, ethylhydroxyethyl cellulose, alginates, carageenans, hyaluronic acid and derivatives thereof, polyacrylic acid, polyvinyl imidazole, polysulphonate, polyethylenglycol/polyethylene oxide, polyethyleneoxide/polypropylene oxide copolymers, polyvinylalcohol/polyvinylacetate of different degree of hydrolysis, and polyvinylpyrrolidone are used for thickening of the solution containing the agent. The polymers may also comprise gelatin or collagen.

Alternatively, the antibody polypeptide may simply be dissolved in saline, water, polyethylene glycol, propylene glycol, ethanol or oils (such as safflower oil, corn oil, peanut oil, cottonseed oil or sesame oil), tragacanth gum, and/or various buffers.

It will be appreciated that the pharmaceutical compositions of the invention may include ions and a defined pH for potentiation of action of the active antibody polypeptide. Additionally, the compositions may be subjected to conventional pharmaceutical operations such as sterilisation and/or may contain conventional adjuvants such as preservatives, stabilisers, wetting agents, emulsifiers, buffers, fillers, etc.

The pharmaceutical compositions according to the invention may be administered via any suitable route known to those skilled in the art. Thus, possible routes of administration include parenteral (intravenous, subcutaneous, and intramuscular), topical, ocular, nasal, pulmonar, buccal, oral, parenteral, vaginal and rectal. Also administration from implants is possible.

In one preferred embodiment, the pharmaceutical compositions are administered parenterally, for example, intravenously, intracerebroventricularly, intraarticularly, intra-arterially, intraperitoneally, intrathecally, intraventricularly, intrasternally, intracranially, intramuscularly or subcutaneously, or they may be administered by infusion techniques. They are conveniently used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Thus, the pharmaceutical compositions of the invention are particularly suitable for parenteral, e.g. intravenous, administration.

Alternatively, the pharmaceutical compositions may be administered intranasally or by inhalation (for example, in the form of an aerosol spray presentation from a pressurised container, pump, spray or nebuliser with the use of a suitable propellant, such as dichlorodifluoromethane, trichlorofluoro-methane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas). In the case of a pressurised aerosol, the dosage unit may be determined by providing a valve to deliver a metered amount. The pressurised container, pump, spray or nebuliser may contain a solution or suspension of the active polypeptide, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

The pharmaceutical compositions will be administered to a patient in a pharmaceutically effective dose. A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art. The administration of the pharmaceutically effective dose can be carried out both by single administration in the form of an individual dose unit or else several smaller dose units and also by multiple administrations of subdivided doses at specific intervals. Alternatively, the dose may be provided as a continuous infusion over a prolonged period.

Particularly preferred compositions are formulated for systemic administration. The composition may preferably be formulated for sustained release over a period of time. Thus the composition may be provided in or as part of a matrix facilitating sustained release. Preferred sustained release matrices may comprise a montanide or γ-polyglutamic acid (PGA) nanoparticles.

The antibody polypeptides can be formulated at various concentrations, depending on the efficacy/toxicity of the polypeptide being used. For example, the formulation may comprise the active antibody polypeptide at a concentration of between 0.1 μM and 1 mM, more preferably between 1 μM and 500 μM, between 500 μM and 1 mM, between 300 μM and 700 μM, between 1 μM and 100 μM, between 100 μM and 200 μM, between 200 μM and 300 μM, between 300 μM and 400 μM, between 400 μM and 500 μM, between 500 μM and 600 μM, between 600 μM and 700 μM, between 800 μM and 900 μM or between 900 μM and 1 mM. Typically, the formulation comprises the active antibody polypeptide at a concentration of between 300 μM and 700 μM.

Typically, the therapeutic dose of the antibody polypeptide (with or without a therapeutic moiety) in a human patient will be in the range of 100 μg to 700 mg per administration (based on a body weight of 70 kg). For example, the maximum therapeutic dose may be in the range of 0.1 to 10 mg/kg per administration, e.g. between 0.1 and 5 mg/kg or between 1 and 5 mg/kg or between 0.1 and 2 mg/kg. It will be appreciated that such a dose may be administered at different intervals, as determined by the oncologist/physician; for example, a dose may be administered daily, twice-weekly, weekly, bi-weekly or monthly.

It will be appreciated by persons skilled in the art that the pharmaceutical compositions of the invention may be administered alone or in combination with other therapeutic agents used in the treatment of cancers, such as antimetabolites, alkylating agents, anthracyclines and other cytotoxic antibiotics, vinca alkyloids, etoposide, platinum compounds, taxanes, topoisomerase I inhibitors, other cytostatic drugs, antiproliferative immunosuppressants, corticosteroids, sex hormones and hormone antagonists, and other therapeutic antibodies (such as antibodies against a tumour-associated antigen or an immune checkpoint modulator).

For example, the pharmaceutical compositions of the invention may be administered in combination with an immunotherapeutic agent that binds a target selected from the group consisting of PD-1/PD-1L, CTLA-4, CD137, CD40, GITR, LAG3, TIM3, CD27, VISTA and KIR.

Thus, the invention encompasses combination therapies comprising a bispecific polypeptide of the invention together with a further immunotherapeutic agent, effective in the treatment of cancer, which specifically binds to an immune checkpoint molecule. It will be appreciated that the therapeutic benefit of the further immunotherapeutic agent may be mediated by attenuating the function of an inhibitory immune checkpoint molecule and/or by activating the function of a stimulatory immune checkpoint or co-stimulatory molecule.

In one embodiment, the further immunotherapeutic agent is selected from the group consisting of:
(a) an immunotherapeutic agent that inhibits the function of PD-1 and/or PD-1L;
(b) an immunotherapeutic agent that inhibits the function of CTLA-4;
(c) an immunotherapeutic agent that activates the function of CD137;
(d) an immunotherapeutic agent that binds activates the function of CD40;
(e) an immunotherapeutic agent that inhibits the function of Lag3;
(f) an immunotherapeutic agent that inhibits the function of Tim3; and
(g) an immunotherapeutic agent that inhibits the function of VISTA.

Thus, the further immunotherapeutic agent may be a PD1 inhibitor, such as an anti-PD1 antibody, or antigen-binding fragment thereof capable of inhibiting PD1 function (for example, Nivolumab, Pembrolizumab, Lambrolizumab, PDR-001, MEDI-0680 and AMP-224). Alternatively, the PD1 inhibitor may comprise or consist of an anti-PD-L1 antibody, or antigen-binding fragment thereof capable of inhibiting PD1 function (for example, Durvalumab, Atezolizumab, Avelumab and MDX-1105).

In another embodiment, the further immunotherapeutic agent is a CTLA-4 inhibitor, such as an anti-CTLA-4 antibody or antigen-binding portion thereof.

In a further embodiment, the further immunotherapeutic agent activates CD137, such as an agonistic anti-CD137 antibody or antigen-binding portion thereof.

In a further embodiment, the further immunotherapeutic agent activates CD40, such as an agonistic anti-CD40 antibody or antigen-binding portion thereof.

In a further embodiment, the further immunotherapeutic agent inhibits the function of Lag3, Tim3 or VISTA (Lines et al. 2014).

It will be appreciated by persons skilled in the art that the presence of the two active agents (as detailed above) may provide a synergistic benefit in the treatment of a tumour in a subject. By “synergistic” we include that the therapeutic effect of the two agents in combination (e.g. as determined by reference to the rate of growth or the size of the tumour) is greater than the additive therapeutic effect of the two agents administered on their own. Such synergism can be identified by testing the active agents, alone and in combination, in a relevant cell line model of the solid tumour.

Also within the scope of the present invention are kits comprising polypeptides or other compositions of the invention and instructions for use. The kit may further contain one or more additional reagents, such as an additional therapeutic or prophylactic agent as discussed above.

Medical Uses and Methods

The polypeptides in accordance with the present invention may be used in therapy or prophylaxis. In therapeutic applications, polypeptides or compositions are administered to a subject already suffering from a disorder or condition, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as “therapeutically effective amount”. In prophylactic applications, polypeptides or compositions are administered to a subject not yet exhibiting symptoms of a disorder or condition, in an amount sufficient to prevent or delay the development of symptoms. Such an amount is defined as a “prophylactically effective amount”. The subject may have been identified as being at risk of developing the disease or condition by any suitable means.

Thus, a seventh aspect of the invention provides a bispecific polypeptide according to the first aspect of the invention for use in medicine.

An eighth aspect of the invention provides a bispecific polypeptide according to the first aspect of the invention for use in treating a neoplastic disorder in a subject.

By ‘treatment’ we include both therapeutic and prophylactic treatment of the patient. The term ‘prophylactic’ is used to encompass the use of an agent, or formulation thereof, as described herein which either prevents or reduces the likelihood of a neoplastic disorder, or the spread, dissemination, or metastasis of cancer cells in a patient or subject. The term ‘prophylactic’ also encompasses the use of an agent, or formulation thereof, as described herein to prevent recurrence of a neoplastic disorder in a patient who has previously been treated for the neoplastic disorder.

In one embodiment, the neoplastic disorder is associated with the formation of solid tumours within the subject's body.

Thus, the solid tumour may be selected from the group consisting of prostate cancer, breast cancer, lung cancer, colorectal cancer, melanomas, bladder cancer, brain/CNS cancer, cervical cancer, oesophageal cancer, gastric cancer, head/neck cancer, kidney cancer, liver cancer, lymphomas, ovarian cancer, pancreatic cancer and sarcomas.

For example, the solid tumour may be selected from the groups consisting of renal cell carcinoma, colorectal cancer, lung cancer, prostate cancer and breast cancer.

A ninth aspect of the invention provides a use of a bispecific polypeptide according to the first aspect of the invention in the preparation of a medicament for treating or preventing a neoplastic disorder in a subject.

In one embodiment, the neoplastic disorder is associated with the formation of solid tumours within the subject's body (for example, as detailed above).

A tenth aspect of the invention provides a method for the treatment or diagnosis of a neoplastic disorder in a subject, comprising the step of administering to the subject an effective amount of a bispecific polypeptide according to the first aspect of the invention.

In one embodiment, the neoplastic disorder is associated with the formation of solid tumours within the subject's body (for example, as detailed above).

In one embodiment, the subject is human.

In one embodiment, the method comprises administering the bispecific antibody systemically.

In one embodiment, the methods further comprises administering to the subject one or more additional therapeutic agents.

The listing or discussion of an apparently prior-published document in this specification should not necessarily be taken as an acknowledgement that the document is part of the state of the art or is common general knowledge.

The use of the word “a” or “an” when used in conjunction with the term “comprising” in the claims and/or the specification may mean “one,” but it is also consistent with the meaning of “one or more,” “at least one,” and “one or more than one.” These, and other, embodiments of the invention will be better appreciated and understood when considered in conjunction with the above description and the accompanying drawings. It should be understood, however, that the above description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

The following drawings form part of the present specification and are included to further demonstrate certain aspects of the present invention. The invention may be better understood by reference to one or more of these drawings in combination with the detailed description of specific embodiments presented herein.

### BRIEF DESCRIPTION OF FIGURES

Preferred, non-limiting examples which embody certain aspects of the invention will now be described, with reference to the following figures:

FIG. 1 shows a schematic representation of the structure of exemplary formats for a bispecific antibody of the invention. In each format, the constant regions are shown as filled light grey; variable heavy chain regions VH1 are shown as chequered black and white; variable light chain regions VL1 are shown as filled white; variable heavy chain regions VH2 are shown as filled black; and variable light chain regions VL2 are shown as white with diagonal lines. OX40 binding domains (binding domain 1) are typically represented as a pair of a chequered black and white domain with a filled white domain (VH1/VNL1); tumour-associated antigen binding domains (binding domain 2) are typically represented as a pair of a filled black domain and a white domain with diagonal lines (VH2N/L2). However, in all of the formats shown, it will be appreciated that binding domains 1 and 2 may be switched. That is, a OX40 binding domain may occur in a position shown in this figure for a tumour-associated antigen domain, and vice versa.

FIG. 2 shows an example of a dose-response experiment of 5T4 antibodies binding to 5T4-transfected B16 cells, analysed by flow cytometry.

FIG. 3 shows flow cytometry data showing normalized mean fluorescence intensity (MFI) of 5T4 mAb binding at a concentration of 2.5 μg/ml to 5T4-transfected B16 cells. The figure shows the mean±SD of the pooled data from four experiments, with 1-4 data points for each antibody, as indicated in Table 2. MFI values were normalised to reference antibody 1628.

FIG. 4 shows dose-response analysis of 5T4 antibody binding to cynomolgus 5T4-transfected CHO cells.

FIG. 5 is an illustration of 5T4 chimeras used for epitope mapping of 5T4 antibodies. A: Each of the indicated domains E1-E7 were replaced by mouse 5T4 sequence in human/mouse chimeras. B: aa 173-420 were replaced by mouse 5T4 sequence.

FIG. 6 is a plot of dissociation rate constant versus association rate constant for exemplary anti-OX40 antibodies, as determined by surface plasmon resonance.

FIG. 7 shows binding of exemplary anti-OX40 antibodies to human OX40 overexpressed on CHO cells, measured by flow cytometry.

FIG. 8 shows the level of IL-2 production by T cells when incubated in vitro with different exemplary anti-OX40 antibodies. The y-axis is the ratio of the top value of IL-2 production by a tested antibody/the top value of a reference antibody. Mean and SEM values from at least 4 donors are shown.

FIG. 9 shows dual binding to both targets of the bispecific antibodies measured by ELISA (coating: OX40-Fc, detection: biotinylated 5T4 and streptavidin-HRP, mean of duplicates is presented).

FIG. 10 shows induction of IL-2 production in CD4 T cells (mean and SEM of two donors) following stimulation with bispecific antibodies or negative control antibodies (combination of monospecific antibodies) in 5T4-coated wells

FIG. 11 shows induction of IL-2 production in CD4 T cells (mean and SEM of two donors) following stimulation with bispecific antibodies or negative control antibody (isotype control) A) 1170/1167-1210/1211 and 1170/1167-1208/1135 or isotype control in 5T4-coated wells B) 1170/1167-1210/1211 and isotype control with or without coated 5T4

FIG. 12 shows IL-2 production of CD4 T cells following stimulation with bispecific antibodies or a combination of monospecific antibodies at 0.5 nM in 5T4 or non-5T4-coated wells. IL-2 values are normalized and presented as fold change versus the isotype control. Mean and SEM of two donors.

FIG. 13 shows binding of the OX40-TAA bispecific antibodies: EpCAM-1168, EGFR-1168 and HER2-1168 to their corresponding antigens using ELISA. EpCAM, EGFR or HER2 were coated on ELISA plates, and bound bispecific OX40-TAA antibodies were detected with biotinylated OX40.

FIG. 14 shows TAA mediated OX40 dependent activation using a CD4 T cell agonist assay and IL2 as a readout. The wells were coated either with the target-TAA of the bsAb or without. The bispecific antibodies were added both to wells with and without TAA. A) EpCAM-1168, B) EGFR-1168 and C) HER2-1168.

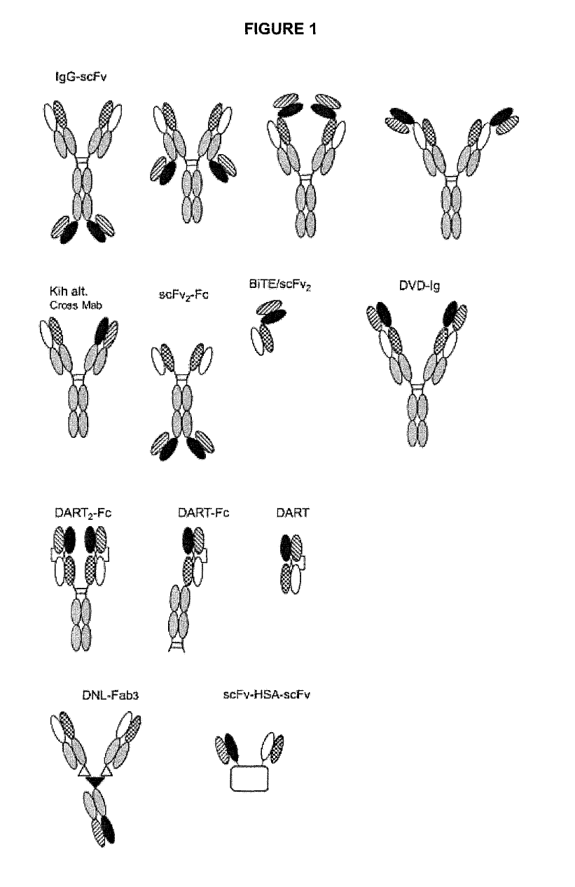

### TABLES (SEQUENCES)

TABLE A
VL and VH amino acid (aa) and nucleotide (nt) sequences

| SEQ | | | |
|---|---|---|---|
| ID | | | |
| NO. | CHAIN NO. | TYPE | SEQUENCE |
| 1 | 1206, heavy | aa | EVQLLESGGGLVQPGGSLRLSCAASGFTFSGSSMSW |
| | chain, VH | | VRQAPGKGLEWVSSIYYSGSGTYYADSVKGRFTISRD |
| | | | NSKNTLYLQMNSLRAEDTAVYYCARYGRNVHPYNLDY |
| | | | WGQGTLVTVSS |
| 2 | 1206, heavy | nt | GAGGTGCAGCTGTTGGAGAGCGGGGGAGGCTTGGT |
| | chain, VH | | ACAGCCTGGGGGGTCCCTGCGCCTCTCCTGTGCAG |
| | | | CCAGCGGATTCACCTTTTCTGGTTCTTCTATGTCTTG |
| | | | GGTCCGCCAGGCTCCAGGGAAGGGGCTGGAGTGG |
| | | | GTCTCATCTATTTACTACTCTGGTTCTGGTACATACT |
| | | | ATGCAGACTCCGTGAAGGGCCGGTTCACCATCTCCC |
| | | | GTGACAATTCCAAGAACACGCTGTATCTGCAAATGA |
| | | | ACAGCCTGCGTGCCGAGGACACGGCTGTATATTATT |
| | | | GTGCGCGCTACGGTCGTAACGTTCATCCGTACAACT |
| | | | TGGACTATTGGGGCCAGGGAACCCTGGTCACCGTC |
| | | | TCCTCA |
| 3 | 1207, light | aa | DIQMTQSPSSLSASVGDRVTITCRASQSISSYLNWYQQ |
| | chain VL | | KPGKAPKLLIYAASSLQSGVPSRFSGSGSGTDFTLTISS |
| | | | LQPEDFATYYCQQGYYYLPTFGQGTKLEIK |
| 4 | 1207, light | nt | GACATCCAGATGACCCAGTCTCCATCCTCCCTGAGC |
| | chain VL | | GCATCTGTAGGAGACCGCGTCACCATCACTTGCCGG |
| | | | GCAAGTCAGAGCATTAGCAGCTATTTAAATTGGTATC |
| | | | AGCAGAAACCAGGGAAAGCCCCTAAGCTCCTGATCT |
| | | | ATGCTGCATCCAGTTTGCAAAGTGGGGTCCCATCAC |
| | | | GTTTCAGTGGCAGTGGAAGCGGGACAGATTTCACTC |
| | | | TCACCATCAGCAGTCTGCAACCTGAAGATTTTGCAA |
| | | | CTTATTACTGTCAACAGGGTTACTACTACCTGCCCAC |
| | | | TTTTGGCCAGGGGACCAAGCTGGAGATCAAA |
| 5 | 1208, heavy | aa | EVQLLESGGGLVQPGGSLRLSCAASGFTFSSYAMSWV |
| | chain VH | | RQAPGKGLEWVSAISGSGGSTYYADSVKGRFTISRDN |
| | | | SKNTLYLQMNSLRAEDTAVYYCARSPYYYGANWIDYW |
| | | | GQGTLVTVSS |
| 6 | 1208, heavy | nt | GAGGTGCAGCTGTTGGAGAGCGGGGGAGGCTTGGT |
| | chain VH | | ACAGCCTGGGGGGTCCCTGCGCCTCTCCTGTGCAG |
| | | | CCAGCGGATTCACCTTTAGCAGCTATGCCATGAGCT |
| | | | GGGTCCGCCAGGCTCCAGGGAAGGGGCTGGAGTG |
| | | | GGTCTCAGCTATTAGTGGTAGTGGTGGTAGCACATA |
| | | | CTATGCAGACTCCGTGAAGGGCCGGTTCACCATCTC |
| | | | CCGTGACAATTCCAAGAACACGCTGTATCTGCAAAT |
| | | | GAACAGCCTGCGTGCCGAGGACACGGCTGTATATTA |
| | | | TTGTGCGCGCTCTCCGTACTACTACGGTGCTAACTG |
| | | | GATTGACTATTGGGGCCAGGGAACCCTGGTCACCGT |
| | | | CTCCTCA |
| 7 | 1135, light | aa | DIQMTQSPSSLSASVGDRVTITCRASQSISSYLNWYQQ |
| | chain VL | | KPGKAPKLLIYAASSLQSGVPSRFSGSGSGTDFTLTISS |
| | | | LQPEDFATYYCQQSYSTPYTFGQGTKLEIK |
| 8 | 1135, light | nt | GACATCCAGATGACCCAGTCTCCATCCTCCCTGAGC |
| | chain VL | | GCATCTGTAGGAGACCGCGTCACCATCACTTGCCGG |
| | | | GCAAGTCAGAGCATTAGCAGCTATTTAAATTGGTATC |
| | | | AGCAGAAACCAGGGAAAGCCCCTAAGCTCCTGATCT |
| | | | ATGCTGCATCCAGTTTGCAAAGTGGGGTCCCATCAC |
| | | | GTTTCAGTGGCAGTGGAAGCGGGACAGATTTCACTC |
| | | | TCACCATCAGCAGTCTGCAACCTGAAGATTTTGCAA |
| | | | CTTATTACTGTCAACAGAGTTACAGTACCCCTTATAC |
| | | | TTTTGGCCAGGGGACCAAGCTGGAGATCAAA |
| 9 | 1210, heavy | aa | EVQLLESGGGLVQPGGSLRLSCAASGFTFSSYAMSWV |
| | chain VH | | RQAPGKGLEWVSAISGSGGSTYYADSVKGRFTISRDN |
| | | | SKNTLYLQMNSLRAEDTAVYYCARYYGGYYSAWMDY |
| | | | WGQGTLVTVSS |
| 10 | 1210, heavy | nt | GAGGTGCAGCTGTTGGAGAGCGGGGGAGGCTTGGT |
| | chain VH | | ACAGCCTGGGGGGTCCCTGCGCCTCTCCTGTGCAG |
| | | | CCAGCGGATTCACCTTTAGCAGCTATGCCATGAGCT |
| | | | GGGTCCGCCAGGCTCCAGGGAAGGGGCTGGAGTG |
| | | | GGTCTCAGCTATTAGTGGTAGTGGTGGTAGCACATA |
| | | | CTATGCAGACTCCGTGAAGGGCCGGTTCACCATCTC |
| | | | CCGTGACAATTCCAAGAACACGCTGTATCTGCAAAT |
| | | | GAACAGCCTGCGTGCCGAGGACACGGCTGTATATTA |
| | | | TTGTGCGCGCTACTACGGTGGTTACTACTCTGCTTG |
| | | | GATGGACTATTGGGGCCAGGGAACCCTGGTCACCG |
| | | | TCTCCTCA |
| 11 | 1211, light | aa | DIQMTQSPSSLSASVGDRVTITCRASQSISSYLNWYQQ |
| | chain VL | | KPGKAPKLLIYAASSLQSGVPSRFSGSGSGTDFTLTISS |
| | | | LQPEDFATYYCQQTYGYLHTFGQGTKLEIK |
| 12 | 1211, light | nt | GACATCCAGATGACCCAGTCTCCATCCTCCCTGAGC |
| | chain VL | | GCATCTGTAGGAGACCGCGTCACCATCACTTGCCGG |
| | | | GCAAGTCAGAGCATTAGCAGCTATTTAAATTGGTATC |
| | | | AGCAGAAACCAGGGAAAGCCCCTAAGCTCCTGATCT |
| | | | ATGCTGCATCCAGTTTGCAAAGTGGGGTCCCATCAC |
| | | | GTTTCAGTGGCAGTGGAAGCGGGACAGATTTCACTC |
| | | | TCACCATCAGCAGTCTGCAACCTGAAGATTTTGCAA |
| | | | CTTATTACTGTCAACAGACTTACGGTTACCTGCACAC |
| | | | TTTTGGCCAGGGGACCAAGCTGGAGATCAAA |
| 13 | 1212, heavy | aa | EVQLLESGGGLVQPGGSLRLSCAASGFTFSSYAMSWV |
| | chain VH | | RQAPGKGLEWVSYISSYGGYTSYADSVKGRFTISRDN |
| | | | SKNTLYLQMNSLRAEDTAVYYCARYHSGVLDYVVGQG |
| | | | TLVTVSS |
| 14 | 1212, heavy | nt | GAGGTGCAGCTGTTGGAGAGCGGGGGAGGCTTGGT |
| | chain VH | | ACAGCCTGGGGGGTCCCTGCGCCTCTCCTGTGCAG |
| | | | CCAGCGGATTCACCTTTAGCAGCTATGCCATGAGCT |
| | | | GGGTCCGCCAGGCTCCAGGGAAGGGGCTGGAGTG |
| | | | GGTCTCATACATTTCTTCTTACGGTGGTTACACATCT |
| | | | TATGCAGACTCCGTGAAGGGCCGGTTCACCATCTCC |
| | | | CGTGACAATTCCAAGAACACGCTGTATCTGCAAATG |
| | | | AACAGCCTGCGTGCCGAGGACACGGCTGTATATTAT |
| | | | TGTGCGCGCTACCATTCTGGTGTTTTGGACTATTGG |
| | | | GGCCAGGGAACCCTGGTCACCGTCTCCTCA |
| 15 | 1213, light | aa | DIQMTQSPSSLSASVGDRVTITCRASQSISSYLNWYQQ |
| | chain VL | | KPGKAPKLLIYAASSLQSGVPSRFSGSGSGTDFTLTISS |
| | | | LQPEDFATYYCQQYYYHYLLTFGQGTKLEIK |
| 16 | 1213, light | nt | GACATCCAGATGACCCAGTCTCCATCCTCCCTGAGC |
| | chain VL | | GCATCTGTAGGAGACCGCGTCACCATCACTTGCCGG |
| | | | GCAAGTCAGAGCATTAGCAGCTATTTAAATTGGTATC |
| | | | AGCAGAAACCAGGGAAAGCCCCTAAGCTCCTGATCT |
| | | | ATGCTGCATCCAGTTTGCAAAGTGGGGTCCCATCAC |
| | | | GTTTCAGTGGCAGTGGAAGCGGGACAGATTTCACTC |
| | | | TCACCATCAGCAGTCTGCAACCTGAAGATTTTGCAA |
| | | | CTTATTACTGTCAACAGTACTACTACCATTACCTGCT |
| | | | CACTTTTGGCCAGGGGACCAAGCTGGAGATCAAA |

TABLE B
5T4 antibody sequences-CDR sequences

| Clone | | | | | | | | |
|---|---|---|---|---|---|---|---|---|
| name | | | | | | | | |
| (mAb) | VH | VL | H1 | H2 | H3 | L1 | L2 | L3 |
| 1206 | 1206 | 1207 | GFTFSG | IYYSGS | ARYGRN | QSISSY | AAS | QQGYYY |
| | | | SS (SEQ | GT (SEQ | VHPYNL | (SEQ ID | (SEQ ID | LPT |
| | | | ID NO: | ID NO: | DY (SEQ | NO: 26) | NO: 27) | (SEQ ID |
| | | | 17) | 19) | ID NO: | | | NO: 28) |
| | | | | | 22) | | | |
| 1208 | 1208 | 1135 | GFTFSS | ISGSGG | ARSPYY | QSISSY | AAS | QQSYST |
| | | | YA (SEQ | ST (SEQ | YGANWI | (SEQ ID | (SEQ ID | PYT |
| | | | ID NO: | ID NO: | DY (SEQ | NO: 26) | NO: 27) | (SEQ ID |
| | | | 18) | 20) | ID NO: | | | NO: 29) |
| | | | | | 23) | | | |
| 1210 | 1210 | 1211 | GFTFSS | ISGSGG | ARYYGG | QSISSY | AAS | QQTYGY |
| | | | YA (SEQ | ST (SEQ | YYSAW | (SEQ ID | (SEQ ID | LHT |
| | | | ID NO: | ID NO: | MDY | NO: 26) | NO: 27) | (SEQ ID |
| | | | 18) | 20) | (SEQ ID | | | NO: 30) |
| | | | | | NO: 24) | | | |
| 1212 | 1212 | 1213 | GFTFSS | ISSYGG | ARYHSG | QSISSY | AAS | QQYYYH |
| | | | YA (SEQ | YT (SEQ | VLDY | (SEQ ID | (SEQ ID | YLLT |
| | | | ID NO: | ID NO: | (SEQ ID | NO: 26) | NO: 27) | (SEQ ID |
| | | | 18) | 21) | NO: 25) | | | NO: 31) |

TABLE C(1)
Exemplary heavy chain CDR sequences (OX40 antibody)

| VH number | SEQ | H CDR1 | SEQ | H CDR2 | SEQ | H CDR3 |
|---|---|---|---|---|---|---|
| 1166 | 32 | GFTFGGYY | 40 | ISGSGGST | 49 | ARYDYASMDY |
| 1170 | | As 1166 | 41 | IPGSGGST | 50 | ARYDYYVVMDY |
| 1164 | 33 | GFTFYGSS | 42 | IYSSGGYT | 51 | ARGVPHGYFDY |
| 1168 | 34 | GFTFSGSS | 43 | ISYYGGYT | 52 | ARYFPHYYFDY |
| 1482 | 35 | GFTFSSYA | 44 | ISYYSGYT | 53 | ARGYGYLDY |
| 1490 | | As 1482 | | As 1168 | 54 | ARYYPHHYIDY |
| 1514 | 36 | GFTFGYYY | 45 | ISSYGSYT | 55 | ARSGYSNWANSFDY |
| 1520 | | As 1482 | | As 1166 | 56 | ARYYYSHGYYVYGTLDY |
| 1524 | 37 | GFTFGSYY | 46 | IGSYYGYT | 57 | ARHDYGALDY |
| 1526 | 38 | GFTFSGYS | 47 | IGYSGYGT | 58 | ARYYFHDYAAYSLDY |
| 1542 | 39 | GFTFGSSS | 48 | IGYYSYSTS | 59 | ARGYPHHYFDY |

TABLE C(2)
Exemplary light chain CDR sequences (OX40 antibody)

| VL number | SEQ | L CDR1 | SEQ | L CDR2 | SEQ | L CDR3 |
|---|---|---|---|---|---|---|
| 1167 | 26 | QSISSY | 27 | AAS | 60 | QQYYWYGLST |
| 1171 | | As 1167 | | As 1167 | 61 | QQGHGSYPHT |
| 1135 | | As 1167 | | As 1167 | 62 | QQSYSTPYT |
| 1483 | | As 1167 | | As 1167 | 63 | QQYGSLLT |
| 1515 | | As 1167 | | As 1167 | 64 | QQGDYTLFT |
| 1525 | | As 1167 | | As 1167 | 65 | QQYGPSGLFT |
| 1527 | | As 1167 | | As 1167 | 66 | QQYGSDSLLT |

TABLE D
Exemplary sequences (OX40 antibody)

| SEQ | | | |
|---|---|---|---|
| ID | | | |
| NO. | CHAIN NO. | TYPE | SEQUENCE |
| 67 | 1167, light chain | aa | DIQMTQSPSSLSASVGDRVTITCRASQSISSYLN |
| | VL | | WYQQKPGKAPKLLIYAASSLQSGVPSRFSGSG |
| | | | SGTDFTLTISSLQPEDFATYYCQQYYWYGLSTF |
| | | | GQGTKLEIK |
| 68 | 1167, light chain | nt | GACATCCAGATGACCCAGTCTCCATCCTCCC |
| | VL | | TGAGCGCATCTGTAGGAGACCGCGTCACCAT |
| | | | CACTTGCCGGGCAAGTCAGAGCATTAGCAGC |
| | | | TATTTAAATTGGTATCAGCAGAAACCAGGGAA |
| | | | AGCCCCTAAGCTCCTGATCTATGCTGCATCC |
| | | | AGTTTGCAAAGTGGGGTCCCATCACGTTTCA |
| | | | GTGGCAGTGGAAGCGGGACAGATTTCACTCT |
| | | | CACCATCAGCAGTCTGCAACCTGAAGATTTTG |
| | | | CAACTTATTACTGTCAACAGTACTACTGGTAC |
| | | | GGTCTGTCCACTTTTGGCCAGGGGACCAAGC |
| | | | TGGAGATCAAA |
| 69 | 1166, heavy | aa | EVQLLESGGGLVQPGGSLRLSCAASGFTFGGY |
| | chain VH | | YMSWVRQAPGKGLEWVSAISGSGGSTYYADS |
| | | | VKGRFTISRDNSKNTLYLQMNSLRAEDTAVYYC |
| | | | ARYDYASMDYWGQGTLVTVSS |
| 70 | 1166, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTGGTGG |
| | | | TTACTACATGTCTTGGGTCCGCCAGGCTCCA |
| | | | GGGAAGGGGCTGGAGTGGGTCTCAGCTATTA |
| | | | GTGGTAGTGGTGGTAGCACATACTATGCAGA |
| | | | CTCCGTGAAGGGCCGGTTCACCATCTCCCGT |
| | | | GACAATTCCAAGAACACGCTGTATCTGCAAAT |
| | | | GAACAGCCTGCGTGCCGAGGACACGGCTGT |
| | | | ATATTATTGTGCGCGCTACGACTACGCTTCTA |
| | | | TGGACTATTGGGGCCAGGGAACCCTGGTCAC |
| | | | CGTCTCCTCA |
| 71 | 1171, light chain | aa | DIQMTQSPSSLSASVGDRVTITCRASQSISSYLN |
| | VL | | WYQQKPGKAPKLLIYAASSLQSGVPSRFSGSG |
| | | | SGTDFTLTISSLQPEDFATYYCQQGHGSYPHTF |
| | | | GQGTKLEIK |
| 72 | 1171, light chain | nt | GACATCCAGATGACCCAGTCTCCATCCTCCC |
| | VL | | TGAGCGCATCTGTAGGAGACCGCGTCACCAT |
| | | | CACTTGCCGGGCAAGTCAGAGCATTAGCAGC |
| | | | TATTTAAATTGGTATCAGCAGAAACCAGGGAA |
| | | | AGCCCCTAAGCTCCTGATCTATGCTGCATCC |
| | | | AGTTTGCAAAGTGGGGTCCCATCACGTTTCA |
| | | | GTGGCAGTGGAAGCGGGACAGATTTCACTCT |
| | | | CACCATCAGCAGTCTGCAACCTGAAGATTTTG |
| | | | CAACTTATTACTGTCAACAGGGTCATGGTTCT |
| | | | TACCCGCACACTTTTGGCCAGGGGACCAAGC |
| | | | TGGAGATCAAA |
| 73 | 1170, heavy | aa | EVQLLESGGGLVQPGGSLRLSCAASGFTFGGY |
| | chain VH | | YMSWVRQAPGKGLEWVSYIPGSGGSTYYADS |
| | | | VKGRFTISRDNSKNTLYLQMNSLRAEDTAVYYC |
| | | | ARYDYYWMDYWGQGTLVTVSS |
| 74 | 1170, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTGGTGG |
| | | | TTACTACATGTCTTGGGTCCGCCAGGCTCCA |
| | | | GGGAAGGGGCTGGAGTGGGTCTCATACATTC |
| | | | CTGGTTCTGGTGGTTCTACATACTATGCAGAC |
| | | | TCCGTGAAGGGCCGGTTCACCATCTCCCGTG |
| | | | ACAATTCCAAGAACACGCTGTATCTGCAAATG |
| | | | AACAGCCTGCGTGCCGAGGACACGGCTGTAT |
| | | | ATTATTGTGCGCGCTACGACTACTACTGGATG |
| | | | GACTATTGGGGCCAGGGAACCCTGGTCACC |
| | | | GTCTCCTCA |
| 75 | 1135, light chain | aa | DIQMTQSPSSLSASVGDRVTITCRASQSISSYLN |
| | VL | | WYQQKPGKAPKLLIYAASSLQSGVPSRFSGSG |
| | | | SGTDFTLTISSLQPEDFATYYCQQSYSTPYTFG |
| | | | QGTKLEIK |
| 76 | 1135, light chain | nt | GACATCCAGATGACCCAGTCTCCATCCTCCC |
| | VL | | TGAGCGCATCTGTAGGAGACCGCGTCACCAT |
| | | | CACTTGCCGGGCAAGTCAGAGCATTAGCAGC |
| | | | TATTTAAATTGGTATCAGCAGAAACCAGGGAA |
| | | | AGCCCCTAAGCTCCTGATCTATGCTGCATCC |
| | | | AGTTTGCAAAGTGGGGTCCCATCACGTTTCA |
| | | | GTGGCAGTGGAAGCGGGACAGATTTCACTCT |
| | | | CACCATCAGCAGTCTGCAACCTGAAGATTTTG |
| | | | CAACTTATTACTGTCAACAGAGTTACAGTACC |
| | | | CCTTATACTTTTGGCCAGGGGACCAAGCTGG |
| | | | AGATCAAA |
| 77 | 1164, heavy | aa | EVQLLESGGGLVQPGGSLRLSCAASGFTFYGS |
| | chain VH | | SMYWVRQAPGKGLEWVSGIYSSGGYTSYADS |
| | | | VKGRFTISRDNSKNTLYLQMNSLRAEDTAVYYC |
| | | | ARGVPHGYFDYWGQGTLVTVSS |
| 78 | 1164, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTTACGG |
| | | | TTCTTCTATGTACTGGGTCCGCCAGGCTCCA |
| | | | GGGAAGGGGCTGGAGTGGGTCTCAGGTATTT |
| | | | ACTCTTCTGGTGGTTACACATCTTATGCAGAC |
| | | | TCCGTGAAGGGCCGGTTCACCATCTCCCGTG |
| | | | ACAATTCCAAGAACACGCTGTATCTGCAAATG |
| | | | AACAGCCTGCGTGCCGAGGACACGGCTGTAT |
| | | | ATTATTGTGCGCGCGGTGTTCCTCATGGTTAC |
| | | | TTTGACTATTGGGGCCAGGGAACCCTGGTCA |
| | | | CCGTCTCCTCA |
| 79 | 1168, heavy | aa | EVQLLESGGGLVQPGGSLRLSCAASGFTFSGS |
| | chain VH | | SMSWVRQAPGKGLEWVSSISYYGGYTYYADS |
| | | | VKGRFTISRDNSKNTLYLQMNSLRAEDTAVYYC |
| | | | ARYFPHYYFDYWGQGTLVTVSS |
| 80 | 1168, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTAGTGG |
| | | | TTCTTCTATGTCTTGGGTCCGCCAGGCTCCA |
| | | | GGGAAGGGGCTGGAGTGGGTCTCATCTATTT |
| | | | CTTACTACGGTGGTTACACATACTATGCAGAC |
| | | | TCCGTGAAGGGCCGGTTCACCATCTCCCGTG |
| | | | ACAATTCCAAGAACACGCTGTATCTGCAAATG |
| | | | AACAGCCTGCGTGCCGAGGACACGGCTGTAT |
| | | | ATTATTGTGCGCGCTACTTCCCGCATTACTAC |
| | | | TTTGACTATTGGGGCCAGGGAACCCTGGTCA |
| | | | CCGTCTCCTCA |
| 81 | 1483, light chain | aa | DIQMTQSPSSLSASVGDRVTITCRASQSISSYLN |
| | VL | | WYQQKPGKAPKLLIYAASSLQSGVPSRFSGSG |
| | | | SGTDFTLTISSLQPEDFATYYCQQYGSLLTFGQ |
| | | | GTKLEIK |
| 82 | 1483, light chain | nt | GACATCCAGATGACCCAGTCTCCATCCTCCC |
| | VL | | TGAGCGCATCTGTAGGAGACCGCGTCACCAT |
| | | | CACTTGCCGGGCAAGTCAGAGCATTAGCAGC |
| | | | TATTTAAATTGGTATCAGCAGAAACCAGGGAA |
| | | | AGCCCCTAAGCTCCTGATCTATGCTGCATCC |
| | | | AGTTTGCAAAGTGGGGTCCCATCACGTTTCA |
| | | | GTGGCAGTGGAAGCGGGACAGATTTCACTCT |
| | | | CACCATCAGCAGTCTGCAACCTGAAGATTTTG |
| | | | CAACTTATTACTGTCAACAGTACGGTTCTCTG |
| | | | CTCACTTTTGGCCAGGGGACCAAGCTGGAGA |
| | | | TCAAA |
| 83 | 1482, heavy | aa | EVQLLESGGGLVQPGGSLRLSCAASGFTFSSY |
| | chain VH | | AMSWVRQAPGKGLEWVSYISYYSGYTYYADSV |
| | | | KGRFTISRDNSKNTLYLQMNSLRAEDTAVYYCA |
| | | | RGYGYLDYWGQGTLVTVSS |
| 84 | 1482, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTAGCAG |
| | | | CTATGCCATGAGCTGGGTCCGCCAGGCTCCA |
| | | | GGGAAGGGGCTGGAGTGGGTCTCATACATTT |
| | | | CTTACTACTCTGGTTACACATACTATGCAGAC |
| | | | TCCGTGAAGGGCCGGTTCACCATCTCCCGTG |
| | | | ACAATTCCAAGAACACGCTGTATCTGCAAATG |
| | | | AACAGCCTGCGTGCCGAGGACACGGCTGTAT |
| | | | ATTATTGTGCGCGCGGTTACGGTTACTTGGA |
| | | | CTATTGGGGCCAGGGAACCCTGGTCACCGTC |
| | | | TCCTCA |
| 85 | 1490, heavy | aa | EVQLLESGGGLVQPGGSLRLSCAASGFTFSSY |
| | chain VH | | AMSWVRQAPGKGLEWVSGISYYGGYTYYADS |
| | | | VKGRFTISRDNSKNTLYLQMNSLRAEDTAVYYC |
| | | | ARYYPHHYIDYWGQGTLVTVSS |
| 86 | 1490, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTAGCAG |
| | | | CTATGCCATGAGCTGGGTCCGCCAGGCTCCA |
| | | | GGGAAGGGGCTGGAGTGGGTCTCAGGTATTT |
| | | | CTTACTACGGTGGTTACACATACTATGCAGAC |
| | | | TCCGTGAAGGGCCGGTTCACCATCTCCCGTG |
| | | | ACAATTCCAAGAACACGCTGTATCTGCAAATG |
| | | | AACAGCCTGCGTGCCGAGGACACGGCTGTAT |
| | | | ATTATTGTGCGCGCTACTACCCGCATCATTAC |
| | | | ATTGACTATTGGGGCCAGGGAACCCTGGTCA |
| | | | CCGTCTCCTCA |
| 87 | 1515, light chain | aa | DIQMTQSPSSLSASVGDRVTITCRASQSISSYLN |
| | VL | | WYQQKPGKAPKLLIYAASSLQSGVPSRFSGSG |
| | | | SGTDFTLTISSLQPEDFATYYCQQGDYTLFTFG |
| | | | QGTKLEIK |
| 88 | 1515, light chain | nt | GACATCCAGATGACCCAGTCTCCATCCTCCC |
| | VL | | TGAGCGCATCTGTAGGAGACCGCGTCACCAT |
| | | | CACTTGCCGGGCAAGTCAGAGCATTAGCAGC |
| | | | TATTTAAATTGGTATCAGCAGAAACCAGGGAA |
| | | | AGCCCCTAAGCTCCTGATCTATGCTGCATCC |
| | | | AGTTTGCAAAGTGGGGTCCCATCACGTTTCA |
| | | | GTGGCAGTGGAAGCGGGACAGATTTCACTCT |
| | | | CACCATCAGCAGTCTGCAACCTGAAGATTTTG |
| | | | CAACTTATTACTGTCAACAGGGTGATTACACT |
| | | | CTGTTCACTTTTGGCCAGGGGACCAAGCTGG |
| | | | AGATCAAA |
| 89 | 1514, heavy | aa | EVQLLESGGGLVQPGGSLRLSCAASGFTFGYY |
| | chain VH | | YMSWVRQAPGKGLEWVSGISSYGSYTYYADS |
| | | | VKGRFTISRDNSKNTLYLQMNSLRAEDTAVYYC |
| | | | ARSGYSNWANSFDYWGQGTLVTVSS |
| 90 | 1514, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTGGTTA |
| | | | CTACTACATGTCTTGGGTCCGCCAGGCTCCA |
| | | | GGGAAGGGGCTGGAGTGGGTCTCAGGTATTT |
| | | | CTTCTTACGGTAGTTACACATACTATGCAGAC |
| | | | TCCGTGAAGGGCCGGTTCACCATCTCCCGTG |
| | | | ACAATTCCAAGAACACGCTGTATCTGCAAATG |
| | | | AACAGCCTGCGTGCCGAGGACACGGCTGTAT |
| | | | ATTATTGTGCGCGCTCTGGTTACTCTAACTGG |
| | | | GCTAACTCTTTTGACTATTGGGGCCAGGGAA |
| | | | CCCTGGTCACCGTCTCCTCA |
| 91 | 1520, heavy | aa | EVQLLESGGGLVQPGGSLRLSCAASGFTFSSY |
| | chain VH | | AMSWVRQAPGKGLEWVSAISGSGGSTYYADS |
| | | | VKGRFTISRDNSKNTLYLQMNSLRAEDTAVYYC |
| | | | ARYYYSHGYYVYGTLDYWGQGTLVTVSS |
| 92 | 1520, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTAGCAG |
| | | | CTATGCCATGAGCTGGGTCCGCCAGGCTCCA |
| | | | GGGAAGGGGCTGGAGTGGGTCTCAGCTATTA |
| | | | GTGGTAGTGGTGGTAGCACATACTATGCAGA |
| | | | CTCCGTGAAGGGCCGGTTCACCATCTCCCGT |
| | | | GACAATTCCAAGAACACGCTGTATCTGCAAAT |
| | | | GAACAGCCTGCGTGCCGAGGACACGGCTGT |
| | | | ATATTATTGTGCGCGCTACTACTACTCTCATG |
| | | | GTTACTACGTTTACGGTACTTTGGACTATTGG |
| | | | GGCCAGGGAACCCTGGTCACCGTCTCCTCA |
| 93 | 1525, light chain | aa | DIQMTQSPSSLSASVGDRVTITCRASQSISSYLN |
| | VL | | WYQQKPGKAPKLLIYAASSLQSGVPSRFSGSG |
| | | | SGTDFTLTISSLQPEDFATYYCQQYGPSGLFTF |
| | | | GQGTKLEIK |
| 94 | 1525, light chain | nt | GACATCCAGATGACCCAGTCTCCATCCTCCC |
| | VL | | TGAGCGCATCTGTAGGAGACCGCGTCACCAT |
| | | | CACTTGCCGGGCAAGTCAGAGCATTAGCAGC |
| | | | TATTTAAATTGGTATCAGCAGAAACCAGGGAA |
| | | | AGCCCCTAAGCTCCTGATCTATGCTGCATCC |
| | | | AGTTTGCAAAGTGGGGTCCCATCACGTTTCA |
| | | | GTGGCAGTGGAAGCGGGACAGATTTCACTCT |
| | | | CACCATCAGCAGTCTGCAACCTGAAGATTTTG |
| | | | CAACTTATTACTGTCAACAGTACGGTCCGTCT |
| | | | GGTCTGTTCACTTTTGGCCAGGGGACCAAGC |
| | | | TGGAGATCAAA |
| 95 | 1524, heavy | aa | EVQLLESGGGLVQPGGSLRLSCAASGFTFGSY |
| | chain VH | | YMGWVRQAPGKGLEWVSSIGSYYGYTYYADS |
| | | | VKGRFTISRDNSKNTLYLQMNSLRAEDTAVYYC |
| | | | ARHDYGALDYWGQGTLVTVSS |
| 96 | 1524, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTGGTTC |
| | | | TTACTACATGGGTTGGGTCCGCCAGGCTCCA |
| | | | GGGAAGGGGCTGGAGTGGGTCTCATCTATTG |
| | | | GTTCTTACTACGGTTACACATACTATGCAGAC |
| | | | TCCGTGAAGGGCCGGTTCACCATCTCCCGTG |
| | | | ACAATTCCAAGAACACGCTGTATCTGCAAATG |
| | | | AACAGCCTGCGTGCCGAGGACACGGCTGTAT |
| | | | ATTATTGTGCGCGCCATGACTACGGTGCTTT |
| | | | GGACTATTGGGGCCAGGGAACCCTGGTCAC |
| | | | CGTCTCCTCA |
| 97 | 1527, light chain | aa | DIQMTQSPSSLSASVGDRVTITCRASQSISSYLN |
| | VL | | WYQQKPGKAPKLLIYAASSLQSGVPSRFSGSG |
| | | | SGTDFTLTISSLQPEDFATYYCQQYGSDSLLTF |
| | | | GQGTKLEIK |
| 98 | 1527, light chain | nt | GACATCCAGATGACCCAGTCTCCATCCTCCC |
| | VL | | TGAGCGCATCTGTAGGAGACCGCGTCACCAT |
| | | | CACTTGCCGGGCAAGTCAGAGCATTAGCAGC |
| | | | TATTTAAATTGGTATCAGCAGAAACCAGGGAA |
| | | | AGCCCCTAAGCTCCTGATCTATGCTGCATCC |
| | | | AGTTTGCAAAGTGGGGTCCCATCACGTTTCA |
| | | | GTGGCAGTGGAAGCGGGACAGATTTCACTCT |
| | | | CACCATCAGCAGTCTGCAACCTGAAGATTTTG |
| | | | CAACTTATTACTGTCAACAGTACGGTTCTGAT |
| | | | TCTCTGCTCACTTTTGGCCAGGGGACCAAGC |
| | | | TGGAGATCAAA |
| 99 | 1526, heavy | aa | EVQLLESGGGLVQPGGSLRLSCAASGFTFSGY |
| | chain VH | | SMYWVRQAPGKGLEWVSGIGYSGYGTYYADS |
| | | | VKGRFTISRDNSKNTLYLQMNSLRAEDTAVYYC |
| | | | ARYYFHDYAAYSLDYWGQGTLVTVSS |
| 100 | 1526, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTTCTGG |
| | | | TTACTCTATGTACTGGGTCCGCCAGGCTCCA |
| | | | GGGAAGGGGCTGGAGTGGGTCTCAGGTATT |
| | | | GGTTACTCTGGTTACGGTACATACTATGCAGA |
| | | | CTCCGTGAAGGGCCGGTTCACCATCTCCCGT |
| | | | GACAATTCCAAGAACACGCTGTATCTGCAAAT |
| | | | GAACAGCCTGCGTGCCGAGGACACGGCTGT |
| | | | ATATTATTGTGCGCGCTACTACTTCCATGACT |
| | | | ACGCTGCTTACTCTTTGGACTATTGGGGCCA |
| | | | GGGAACCCTGGTCACCGTCTCCTCA |
| 101 | 1542, heavy | aa | EVQLLESGGGLVQPGGSLRLSCAASGFTFGSS |
| | chain VH | | SMYWVRQAPGKGLEWVSGIGYYSYSTSYADS |
| | | | VKGRFTISRDNSKNTLYLQMNSLRAEDTAVYYC |
| | | | ARGYPHHYFDYWGQGTLVTVSS |
| 102 | 1542, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTGGTTC |
| | | | TTCTTCTATGTACTGGGTCCGCCAGGCTCCA |
| | | | GGGAAGGGGCTGGAGTGGGTCTCAGGTATT |
| | | | GGTTACTACTCTTACTCTACATCTTATGCAGA |
| | | | CTCCGTGAAGGGCCGGTTCACCATCTCCCGT |
| | | | GACAATTCCAAGAACACGCTGTATCTGCAAAT |
| | | | GAACAGCCTGCGTGCCGAGGACACGGCTGT |
| | | | ATATTATTGTGCGCGCGGTTACCCGCATCATT |
| | | | ACTTTGACTATTGGGGCCAGGGAACCCTGGT |
| | | | CACCGTCTCCTCA |

Mutated IgG1 Antibody Sequence

IgG1 LALA-sequence
(SEQ ID NO: 103)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISMKGQPREPQVYTLPPSRDELTKNQVSLTCL
VKGFYPSDIAVEWESNGQPENNYKTTPPVLDSDGSFFLYSKLTVDKSRWQ
QGNVFSCSVMHEALHNHYTQKSLSLSPGK

Linker Sequences

(SEQ ID NO: 104)
SGGGGSGGGGS
(SEQ ID NO: 105)
SGGGGSGGGGSAP
(SEQ ID NO: 106)
NFSQP
(SEQ ID NO: 107)
KRTVA
(SEQ ID NO: 108)
GGGSGGGG
(SEQ ID NO: 109)
GGGGSGGGGS
(SEQ ID NO: 110)
GGGGSGGGGSGGGGS
(SEQ ID NO: 116)
GSTSGSGKPGSGEGSTKG
(SEQ ID NO: 117)
THTCPPCPEPKSSDK
(SEQ ID NO: 118)
GGGS
(SEQ ID NO: 119)
EAAKEAAKGGGGS
(SEQ ID NO: 120)
EAAKEAAK
(SG)m, where m = 1 to 7.

IgG Constant Region Sequences

IgG1 heavy chain constant region sequence:
[SEQ ID NO: 111]
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
IgG1 light chain constant region sequence:
[SEQ ID NO: 112]
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC
Modified IgG4 heavy chain constant region sequence
[SEQ ID NO: 113]
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNRYTQKSLSLSLGK
Modified IgG4 heavy chain constant region sequence
[SEQ ID NO: 114]
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
Wild type IgG4 heavy chain constant region
sequence
[SEQ ID NO: 115]
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK

### EXAMPLES

### Example 1—Selection of 5T4 Antibodies from Alligator-GOLD™ Library

Phage display selections against h5T4 were performed using the scFv library ALLIGATOR-GOLD™, a fully human scFv library containing more than 1×10¹⁰ unique members (Alligator Bioscience AB, Lund, Sweden). Several different selection strategies were employed, including solid phase selection, selection in solution using biotinylated 5T4-Fc, selection with biotinylated 5T4-Fc coupled to streptavidin beads as well as one round of selection against 5T4 expressing B16 cells using a phage stock that previously had been selected against the recombinant h5T4-Fc. Prior to selection, phage stocks were pre-selected against streptavidin, Beriglobin or SLIT2 in order to remove potential binders to streptavidin, the Fc part of the target and binders cross reactive to other leucine rich repeat proteins.

To identify specific binders from the phage selection, approximately 1250 individual clones were screened in phage format using ELISA coated with 5T4-Fc or non-target protein (Biglycan or Orencia). This was followed by sequence analysis as well as screening as soluble scFv in full-curve ELISA, ELISA performed at 50° C. and FACS analysis of selected clones. Based on this, 14 unique candidate scFv were chosen which bound to recombinant 5T4 and to 5T4 expressing cells without showing positive response to non-target molecules or to 5T4 negative cells.

The selected 14 5T4 scFv clones were converted to full IgG1 for further characterization. A reference anti-5T4 antibody, designated 1628 (selected from a representative prior art disclosure), was used in this study as a positive control.

Among the 14 clones, four clones were selected for further evaluation in bispecific antibody format. These four clones are described further below, and compared to the reference clone 1628.

### Example 2—Binding to Human 5T4 Measured by ELISA

Materials and Methods

ELISA was performed using a standard protocol. Plates (#655074, Greiner Bio-One GmbH, Germany) were pre-coated with 0.5 μg/ml 5T4-Fc (obtained from Peter L. Stern, University of Manchester) overnight. 5T4 antibodies were diluted from 6 to 1.5×10⁻³ μg/ml in 1:4 dilutions and added in duplicates of 50 μl to each well. Binding was detected with rabbit anti-h kappa L-chain-HRP (P0129, Dako Denmark) and the ELISA was developed with SuperSignal ELISA PICO Chemiluminescent substrate (Thermo Scientific Pierce, Rockford, Ill. USA) for 2-10 minutes and read in an automated microplate based multi-detection reader (FLUOstar OPTIMA, Netherlands).

Results and Conclusions

The results show that the majority of the 5T4 mAbs bind with similar potency to 5T4 as 1628 (Table 1) with EC50 values in the sub-nM range. However, clone 1208 exhibits a slightly higher EC50 value.

TABLE 1
Summary of the obtained EC50 in the ELISA of all 5T4 mAb
with the confidence intervals and number of experiments

| | | EC50 (nM) | |
|---|---|---|---|
| Clone name | Mean EC50 (nM) | 95% Confidence Intervals | n |
| Reference 1628 | 0.56 | 0.3-1.0 | 8 |
| 1206 | 0.64 | 0.3-1.4 | 4 |
| 1208 | 2.24 | 2.0-2.4 | 1 |
| 1210 | 0.48 | 0.2-1.1 | 4 |
| 1212 | 0.56 | 0.2-1.2 | 4 |

### Example 3—Binding to 5T4 Expressed on the Cell Surface Determined by Flow Cytometry

Materials and Methods

Analysis of 5T4 mAb binding with flow cytometry was performed using 5T4-transfected cell lines and as negative control, mock transfected cells. Three different transfected cell lines used were used for this study; B16, A9 and CHO, transfected either with a 5T4 construct or with an empty vector control construct. Cells were stained with 5T4 antibodies diluted in FACS buffer (PBS, 0.5% BSA and 0.02% NaN₃). Binding was detected with the secondary antibody anti-IgG (Fc)-PE (109-115-098, Jackson ImmunoResearch Europe, UK) diluted 1:100. Samples were analysed either on a FACSCalibur or a FACSverse (BD Biosciences, Heidelberg, Germany) and mean fluorescence intensity (MFI) determined.

Results and Conclusions

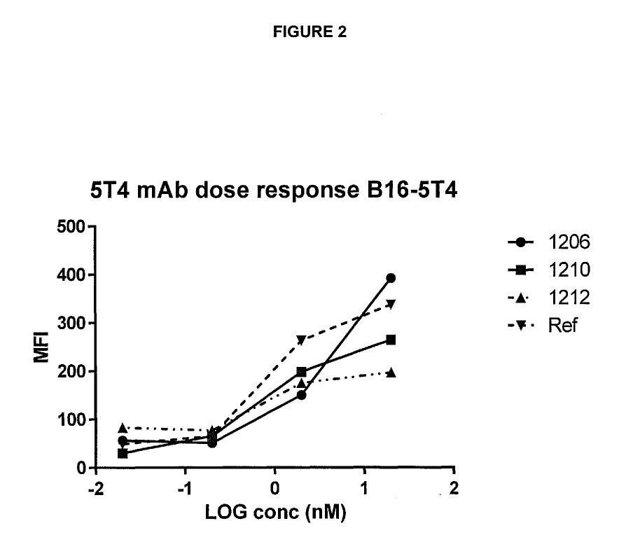

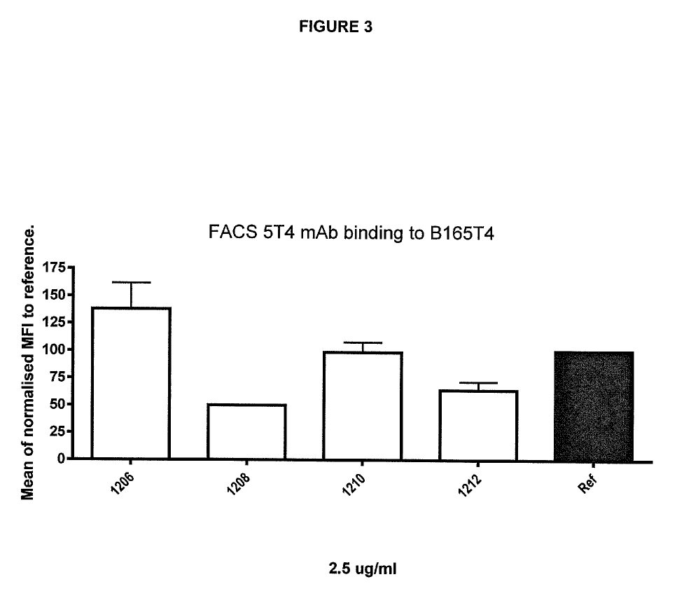

In flow cytometric analysis of 5T4 antibody binding to 5T4-transfected B16 cells, most antibodies show good binding. Large variations in EC50 values between individual experiments were observed. Therefore, results are summarized as mean EC50 in nM as well as mean EC50 normalized to the internal control 1628 (Table 2). An example of dose-response curves for binding of 5T4 mAb to 5T4-transfected B16 cells is shown in FIG. 2. In FIG. 3, normalized MFI values at a fixed concentration of 2.5 μg/ml antibody is shown. Taken together, the data indicate that most antibodies bind well to 5T4-transfected B 16 cells, with clone1208 exhibiting weaker binding.

TABLE 2
Potency of 5T4 antibodies as determined by flow
cytometric analysis of 5T4-transfected B16 cells

| | EC50 (nM) | | Normalized EC50* | | |
|---|---|---|---|---|---|
| Clone | Mean | SD | Mean | SD | n |
| 1206 | 1.8 | 1.6 | 3.9 | 2.7 | 4 |
| 1208 | 0.7 | | 9.3 | | 1 |
| 1210 | 0.8 | 0.7 | 1.6 | 1.3 | 4 |
| 1212 | 1.4 | 2.2 | 1.1 | 0.3 | 4 |
| Reference 1628 | 1.1 | 1.4 | 1.0 | | 4 |

*EC50 value normalized to 1628

In a new attempt to calculate EC50 with flow cytometry a 5T4 mAb dose response experiment was performed using CHO cells stably transfected with human 5T4. A one to four titration series was performed starting from 2.5 nM. The data are summarized in Table 3.

TABLE 3
Summary of EC50 values, EC50 95% confidence
intervals and EC50 normalised to 1628 in flow
cytometric analysis of 5T4-transfected CHO cells.
Data was normalised and the EC50
values were calculated by nonlinear regression.

| | 1206 | 1208 | 1210 | 1628 |
|---|---|---|---|---|
| EC50 nM | 0.51 | 2.07 | 0.81 | 0.51 |
| EC50 (95% | 0.2 to 1.2 | 1.6. to 2.7 | 0.3 to 2.2 | 0.14 to 1.8 |
| confidence intervals) | | | | |
| Normalized to | 1.0 | 4.1 | 1.6 | 1.0 |
| reference 1628 | | | | |

Finally, binding potency to 5T4-transfected A9 cells was evaluated in two individual experiments. As in the experiments performed with B16-5T4 cells, the absolute EC50 values determined in individual experiments vary, and data is therefore presented as normalized to the reference 1628 (Table 4). Results indicate that the 5T4 antibodies bind with comparable potency to the reference 1628.

TABLE 4
Potency of 5T4 antibodies as determined by flow
cytometric analysis of 5T4-transfected A9 cells

| | Normalized EC50* | | |
|---|---|---|---|
| Clone | Mean | SD | n |
| 1206 | 2.9 | 1.9 | 2 |
| 1208 | 3.9 | 2.2 | 2 |
| 1210 | 1.8 | 0.2 | 2 |
| 1212 | 0.4 | — | 1 |
| 1628 | 1.0 | — | 2 |

*EC50 value normalized to reference 1628

To summarize, the binding potency of four 5T4 antibodies was evaluated by flow cytometry using three different 5T4-transfected cell lines (B16, CHO and A9). The conclusion from these studies is that all antibodies exhibit reasonable binding, with clone 1208 in general exhibiting lower potency than the other clones.

### Example 4—Binding to Cynomolgus 5T4

Materials and Methods

The potency of 5T4 antibodies in binding to cynomolgus 5T4 was determined by flow cytometry. CHO cells were stably transfected with Macaca mulatta (cynomolgus) 5T4. Cells were stained with 5T4 antibodies diluted in FACS buffer (PBS, 0.5% BSA and 0.02% NaN₃) using a 1:4 titration starting at 2.5 nM. Binding was detected with the secondary antibody anti-IgG (Fc)-PE (109-115-098, Jackson ImmunoResearch Europe, UK) diluted 1:100. Samples were analysed either on a FACSCalibur or a FACSverse (BD Biosciences, Heidelberg, Germany) and mean fluorescence intensity (MFI) determined. Three experiments were performed with comparable results, although only one experiment included a full dose-response curve whereas the other two experiments included only three antibody concentrations. To compare the EC50 values between human and cynomolgus 5T4, the cy5T4/hu5T4 ratio was calculated from the experiment with the full dose-response.

Results and Conclusions

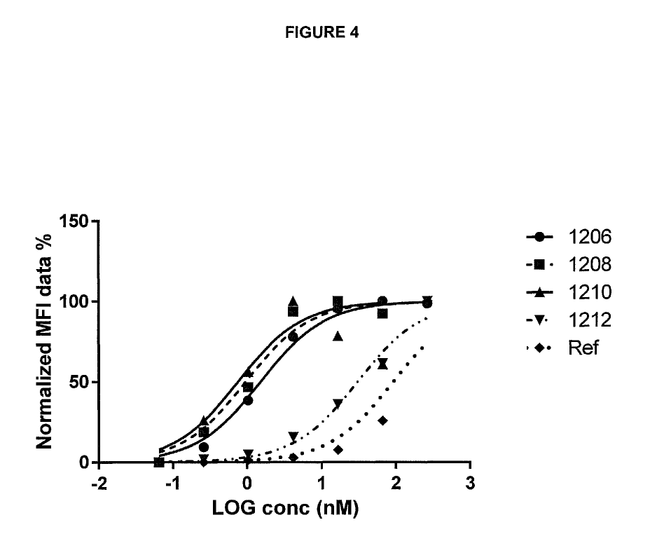

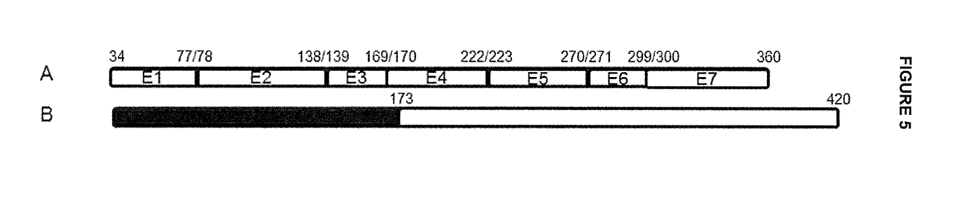

The three experiments that were performed demonstrate good binding to cynomolgus 5T4 by clones 1206, 1208 and 1210 and weak binding by 1212 and the reference 1628 (FIG. 4, Table 5) Clone 1206 had a relatively good potency, but low efficacy. Comparison of the relative EC50 values between cynomolgus and human 5T4 for selected clones shows that clones 1206, 1208 and 1210 have a relatively high affinity for cynomolgus 5T4 whereas 1212 does not.

TABLE 5
EC50 values for cyno5T4 transfected cells and
EC50 95% confidence intervals and the EC50cyno:EC50 human

| | Antibody | | | | |
|---|---|---|---|---|---|
| | 1206 | 1208 | 1210 | 1212 | 1628 |
| EC50 nM | 1.53 | 0.96 | 0.70 | 30.7 | 93.0 |
| EC50 (95% | 1.1 to 2.1 | 0.5 to 1.8 | 0.3 to 1.7 | 21 to 45 | 37 to 235 |
| confidence | | | | | |
| intervals) | | | | | |
| Ratio | 3.0 | 0.5 | 0.9 | 140 | 182 |
| EC50cyno- | | | | | |
| 5T4/h5T4 | | | | | |

Data were normalised and the EC50 values were calculated by nonlinear regression

### Example 5—Affinity Determined by Surface Plasmon Resonance

Materials and Methods

Binding kinetics of the 5T4-specific mAbs have been studied using two different SPR-based platforms, the Biacore 3000 (GE Healthcare) and the MASS-1 platform (Sierra Sensors). Briefly, 5T4 was captured at the sensor chip surface either via direct amine coupling (Biacore platform) or using a streptavidin coated chip and biotinylated 5T4 (MASS-1 platform). The different 5T4-specific mAbs were then injected over the chip in increasing concentrations and the association and dissociation rates studied in real time. A 1:1 Langmuir model was used for curve fitting.

Results and Conclusions

A summary of binding rate constants and affinities obtained using the two platforms is presented in Table 6. It should however be taken into consideration that the assay setup used allows for bivalent binding of the mAbs to the antigen. This will give rise to avidity effects that lead to a significant underestimation of the off-rates (kd) and thus also the affinity value (KD). The different 5T4 antibodies show different binding characteristics, with 1208 and the reference 1628 displaying very low off-rates while on-rates vary less between the binders. It is obvious that there are significant variations between the two assays, with an over 10-fold difference for 1206 and 1628. For 1628 this is likely due the difficulty in accurate curve fitting when the off-rate becomes very low (close to no dissociation).

TABLE 6
Summary of binding kinetics of 5T4-specific mAbs

| | Biacore | | | MASS-1 | | |
|---|---|---|---|---|---|---|
| Clone | ka (1/Ms) | kd (1/s) | KD (M) | ka (1/Ms) | kd (1/s) | KD (nM) |
| 1206 | 1.3E+05 | 2.8E−04 | 2.3E−09 | 1.4E+06 | 1.3E−04 | 9.7E−11 |
| 1208 | — | — | — | 2.1E+05 | 2.1E−06 | 9.8E−12 |
| 1210 | 5.0E+05 | 1.0E−04 | 2.0E−10 | 5.1E+05 | 1.9E−04 | 3.7E−10 |
| 1212 | 4.5E+05 | 8.1E−04 | 1.8E−10 | — | — | — |
| 1628 | 6.4E+05 | 2.6E−08 | 4.1E−14 | 1.5E+06 | 2.1E−06 | 1.5E−12 |

### Example 6—Domain Mapping of 5T4 Antibodies

Materials and Methods

Epitope mapping was performed by investigation of loss of binding by the antibodies using a panel of human/mouse chimeric 5T4 constructs by flow cytometry. This strategy was possible since none of the 5T4 antibodies cross-react with murine 5T4. Two strategies were used for the epitope mapping as illustrated in FIG. 5. In one approach, seven human/mouse 5T4 chimeras were constructed based on dividing 5T4 into seven different domains (FIG. 5). By replacing each domain with the corresponding mouse sequence seven human/mouse 7 5T4 human/mouse chimeras were generated. The chimeras were generated using the human protein 5T4 sequence NP_006661.1 (reference mRNA sequence NM_006670.4) and the corresponding mouse sequence NP_035757.2 (reference mRNA sequence NM_011627.4). The human/mouse chimeric DNA constructs, as well as human and mouse wild-type 5T4, were cloned into pcDNA3.1 expression vectors. Stably transfected CHO cells were generated and 5T4 expressing cells enriched by MACS sorting, resulting in 60-80% positive cells. In the other approach, cells transfected with a human/mouse 5T4 chimera (Woods et al., 2002) was used, in which mouse sequence in amino acid 173-420 replaced the human sequence (FIG. 5). As controls human 5T4 and mouse 5T4-transfected cells were used.

For flow cytometric analysis, cells were stained with different 5T4 antibodies diluted in FACS buffer (PBS, 0.5% BSA). Binding was detected with the secondary antibody anti-IgG (Fc)-PE (109-115-098, Jackson ImmunoResearch Europe, UK) diluted 1:100. Samples were analysed by FACSverse (BD Biosciences, Heidelberg, Germany) and % positive cells were determined. To compensate for variations in % 5T4 positive cells in the various transfected populations, binding levels were normalized within each chimera by dividing % positive cells for each clone with % positive cells for the clones resulting in the highest % positive cells (% pos cells_{clonX}/% positive cellsₘₐₓ). A normalized value≤0.75 was defined as mAb binding being dependent on the replaced region, whereas a normalized value≤0.25 was defined as complete dependence.

Results and Conclusion

The four 5T4 antibodies were shown to be more or less dependent on at least one of domains E2, E3, E4, E6 or aa 173-420, whereas no clear dependence on E1, E5 or E7 was observed (Table 7).

All four antibodies had a distinct binding pattern:
1. Clone 1208; dependent on E2 and E4
2. Clone 1210; dependent on E2, E4 and aa173-420
3. Clone 1206; dependent on E2, E3, E4 and aa173-420
4. Clone 1212 dependent on E6 aa173-420

The reference antibody 1628 differed from all the exemplary antibodies of the invention, and was completely dependent on E4 and aa173-420.

TABLE 7
Summary of epitope mapping results summarized as normalized
values for one representative experiment.

| Clone | Group | E1 | E2 | E3 | E4 | E5 | E6 | E7 | aa173-420 |
|---|---|---|---|---|---|---|---|---|---|
| 1628 | | 0.96 | 1.00 | 1.00 | 0.06 | 0.96 | 0.81 | 0.91 | 0.00 |
| 1208 | 1 | 0.95 | 0.68 | 0.96 | 0.65 | 1.00 | 0.94 | 0.99 | 1.00 |
| 1210 | 2 | 0.99 | 0.02 | 0.90 | 0.69 | 0.89 | 0.89 | 0.96 | 0.00 |
| 1206 | 3 | 0.96 | 0.74 | 0.25 | 0.52 | 0.90 | 0.94 | 0.94 | 0.30 |
| 1212 | 4 | 0.89 | 0.90 | 0.93 | 1.00 | 0.84 | 0.03 | 0.88 | −0.01 |

The experiment was repeated once for E2, E3 and E6 chimeras and three times for the E4 chimera with high reproducibility. mAbs with a normalized binding value≤0.75 are indicated in bold.

### Example 7—Characterisation of OX40 Antibodies

Characteristics of exemplary OX40 antibodies are summarised in Table 8 below.

TABLE 8
Characteristics of exemplary OX40 antibodies

| | | Dose | | | huOX40 | | | | | |
|---|---|---|---|---|---|---|---|---|---|---|
| | CDR H3 | response | | M. mulatta | binding | | | Dissociation | Association | Association |
| | length | ELISA | T-cell | OX40 | FACS | Hydrophathy | Isoelectric | constant K_{D} | rate constant | rate constant |
| Antibody | (IMGT) | (IgG) | agonist | binding | (CHO) | index | point | (M) | ka (1/Ms) | kd (1/s) |
| 1166/1167 | 10 | <1 nM | Yes | Yes | Yes | −0.392 | 9.11 | 3.22E−10 | 9.01E+04 | 2.90E−05 |
| 1170/1171 | 10 | <1 nM | Yes | Yes | Yes | −0.415 | 9.11 | 2.50E−10 | 1.45E+06 | 3.63E−04 |
| 1164/1135 | 11 | <1 nM | Yes | Yes | Yes | −0.398 | 9.21 | 3.08E−10 | 2.51E+05 | 7.73E−05 |
| 1168/1135 | 11 | <1 nM | Yes | Yes | Yes | −0.404 | 9.19 | 3.27E−10 | 5.18E+05 | 1.69E−04 |
| 1482/1483 | 9 | <1 nM | Yes | Yes | Yes | −0.381 | 9.19 | 7.53E−10 | 7.76E+05 | 5.84E−04 |
| 1490/1135 | 11 | <1 nM | Yes | Yes | Yes | −0.407 | 9.18 | 3.07E−10 | 3.87E+06 | 1.19E−03 |
| 1514/1515 | 14 | <1 nM | Yes | Yes | Yes | −0.399 | 9.11 | 6.40E−10 | 2.57E+05 | 1.64E−04 |
| 1520/1135 | 17 | <1 nM | Yes | Yes | Yes | −0.394 | 9.18 | 5.55E−10 | 6.20E+05 | 3.44E−04 |
| 1524/1525 | 10 | <1 nM | Yes | Yes | Yes | −0.391 | 9.11 | 8.11E−10 | 1.71E+06 | 1.39E−03 |
| 1526/1527 | 15 | <1 nM | Yes | Yes | Yes | −0.388 | 8.99 | 4.30E−10 | 4.35E+05 | 1.87E−04 |
| 1542/1135 | 11 | <1 nM | Yes | Yes | Yes | −0.411 | 9.2 | 4.63E−10 | 2.16E+05 | 1.00E−04 |

Two anti-OX40 antibodies were synthesised solely for use as reference antibodies for the purposes of comparison in these studies. They are designated herein as “72” or “72/76”, and “74” or “74/78”, respectively.

Measurement of Kinetic Constants by Surface Plasmon Resonance

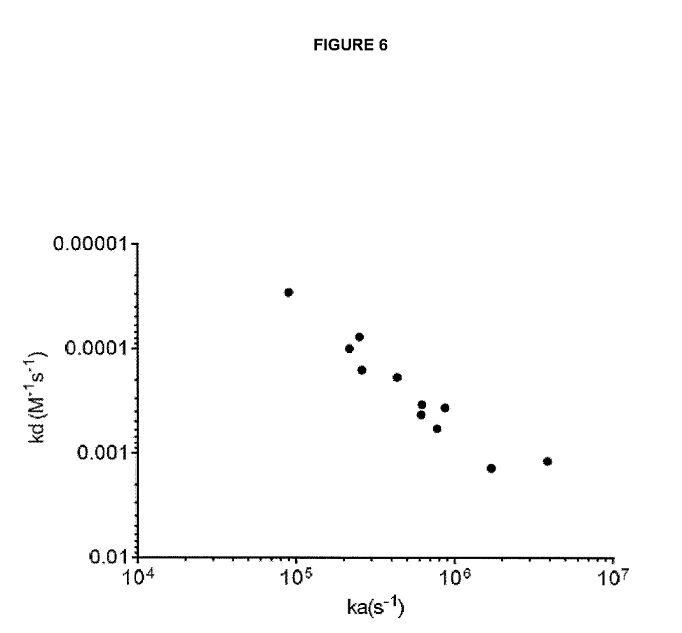

Human OX40 (R&D systems, #3358_OX) was immobilized to the Biacore™ sensor chip, CM5, using conventional amine coupling. The tested antibodies and controls (serially diluted 1/3 or 1/2 100-2 nM) were analyzed for binding in HBS-P (GE, #BR-1003-68) at a flow rate of 30 μl/ml. The association was followed for 3 minutes and the dissociation for 20 minutes. Regeneration was performed twice using 50 mM NaOH for 60 seconds. The kinetic parameters and the affinity constants were calculated using 1:1 Langmuir model with drifting baseline. The tested antibodies were overall in the subnanomolar-nanomolar range with varying on and off rates (FIG. 6 and Table 8). Most of the antibodies had affinities<5 nM. The kinetic parameters and the affinity constants were calculated using BIAevaluation 4.1 software.

Measurement by ELISA of Binding to Human and Murine OX40, and to CD137 and CD40 by ELISA

ELISA plates were coated with human OX40 (R&D Systems, 3388-OX), CD40 (Ancell), or CD137 (R&D Systems) at 0.1 or 0.5 μg/ml. The ELISA plates were washed with PBST and then blocked with PBST+2% BSA for 1 h at room temperature and then washed again with PBST. The antibodies were added in dilution series to the ELISA plates for 1 h at room temperature and then washed with PBST. Binding was detected using goat anti human kappa light chain HRP, incubated for 1 h at room temperature. SuperSignal Pico Luminescent was used as substrate and luminescence was measured using Fluostar Optima.

All the tested OX40 antibodies bound to human OX40 and displayed EC50 value below 1 nM. The antibodies did not bind to murine OX40 or to the other TNFR super family members tested (data not shown).

Measurement of Binding to Human OX40 Over-Expressed on CHO Cells

The extracellular part of human OX40 was fused to the transmembrane and intracellular part of hCD40 and cloned into pcDNA3.0. The vector was subsequently stably transfected into CHO cells. Expression of OX40 was confirmed by incubating with commercial OX40 antibody (huOX40, BD Biosciences) for 30 min at 4° C. and then detected with a-huIgG-PE (Jackson Immunoresearch) 30 min 4° C. For the assay, the transfected cells were incubated with the test antibodies and controls for 30 min at 4° C. and then detected with a-huIgG-PE (Jackson Immunoresearch) 30 min 4° C. Cells were analyzed by flow cytometry with FACS Verse (BD Biosciences).

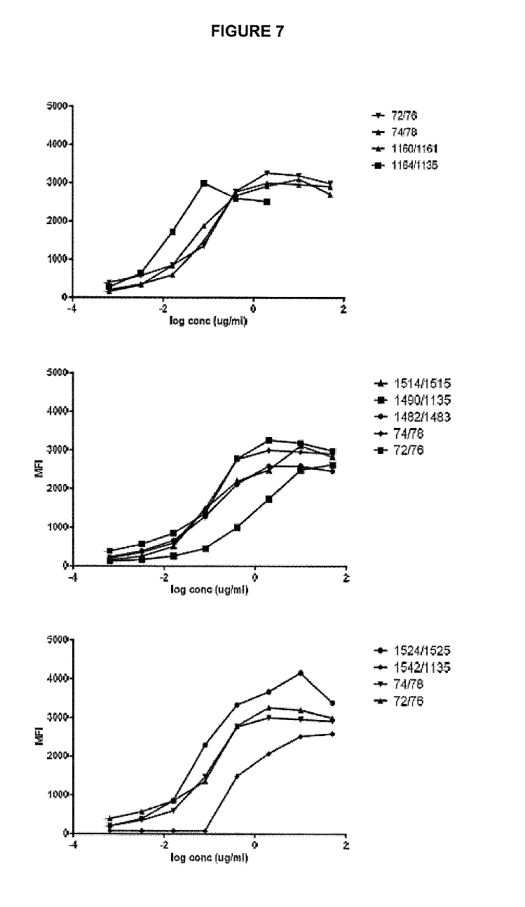

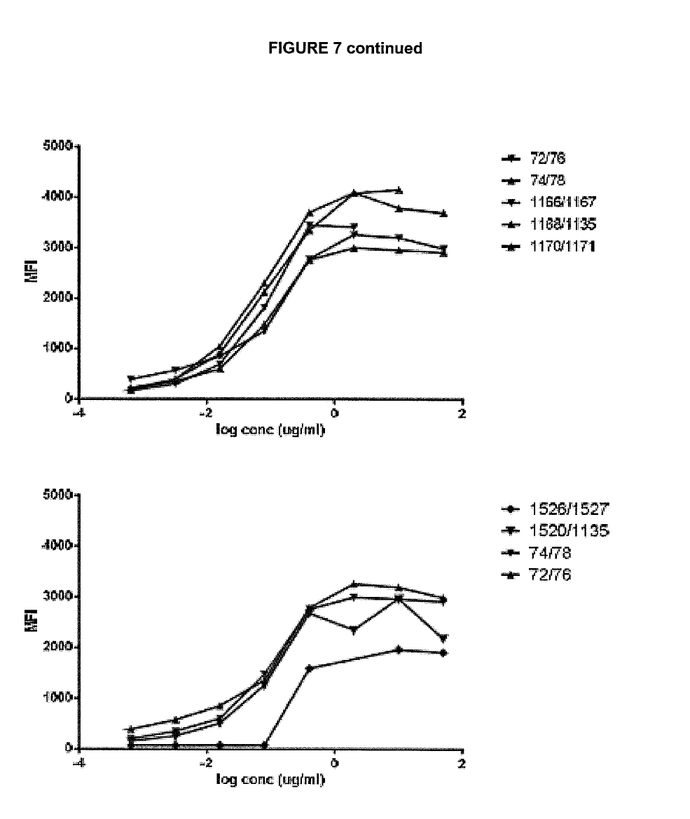

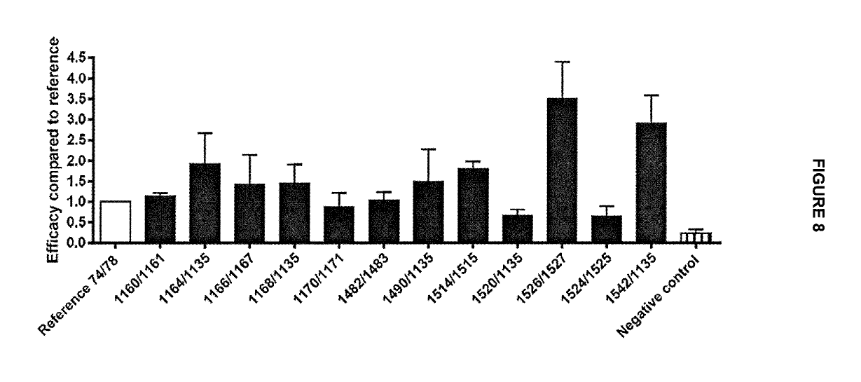

All clones bound to hOX40 overexpressed on CHO cells in a dose dependent manner (FIG. 7).

### Example 8—Sequence Analysis of OX40 Antibodies

The CDR sequences of both the heavy and light chain variable regions were analysed for each antibody. Table 9 illustrates the analysis as conducted for the VH CDR3 sequences. Positions in Table 9 are defined according to IMGT numbering system. The following patterns were identified.

The VH regions all comprise:

(a) a heavy chain CDR1 sequence which is 8 amino acids in length and comprises the consensus sequence: “G, F, T, F, G/Y/S, G/Y/S, Y/S, Y/S/A”;

(b) a heavy chain CDR2 sequence which is 8 amino acids in length and comprises the consensus sequence: “I, G/Y/S/T, G/S/Y, S/Y, G/S/Y, G/S/Y, G/S/Y, T”; and

(c) a heavy chain CDR3 sequence which is 9 to 17 amino acids in length and which comprises the consensus sequence of: “A, R, G/Y/S/H, G/Y/F/V/D, G/Y/P/F, -/H/S, -/N/D/H, -/Y/G, -/Y, -/Y, -/W/A/V, -/A/Y, -/D/A/Y/G/H/N, Y/S/W/A/T, L/M/I/F, D, Y”.

The VL regions all comprise:

(a) a light chain CDR1 sequence which consists of the sequence: “Q, S, I, S, S, Y”;

(b) a light chain CDR2 sequence which consists of the sequence: “A, A, S”;

(c) a light chain CDR3 sequence which is 8 to 10 amino acids in length and comprises the consensus sequence: “Q,Q, S/Y/G, -/Y/H/G, -/S/Y/G/D, S/Y/G/D, S/Y/G/T, P/L, Y/S/H/L/F, T”.

Within the consensus sequence for the heavy chain CDR3, two sub-families were identified. Each antibody in the first sub-family comprises a VH CDR3 sequence of 10 amino acids in length which comprises the consensus sequence “A, R, Y/H, D, Y, A/Y/G, S/W/A, M/L, D, Y”. Antibodies in this family are referred to as family Z and are identified as such in Table 9. Each antibody in the second sub-family comprises a VH CDR3 sequence of 11 amino acids in length which comprises the consensus sequence “A, R, G/Y, V/F/Y, P, H, G/Y/H, Y, F/I, D, Y”. Antibodies in this family are referred to as family P and are identified as such in Table 9. Antibodies of family Z or P are preferred. Antibodies having a VH sequence in family P typically also include a VL sequence with a CDR3 sequence of “Q, Q, S, Y, S, T, P, Y, T”, a CDR1 sequence “Q,S,I,S,S,Y” and a CDR2 sequence of “A,A,S”. Accordingly antibodies with a VL region comprising these three CDR sequences are preferred.

TABLE 9
Analysis conducted for the VH CDR3 sequences

| | | | | | | | | | | | | | | | | | | CDRH3 | FAM- |
|---|---|---|---|---|---|---|---|---|---|---|---|---|---|---|---|---|---|---|---|
| VH | 105 | 106 | 107 | 108 | 109 | 110 | 111 | 111.1 | 111.2 | 112.2 | 112.1 | 112 | 113 | 114 | 115 | 116 | 117 | LENGTH | ILY |
| 1482 | A | R | G | Y | G | | | | | | | | | Y | L | D | Y | 9 | |
| 1166 | A | R | Y | D | Y | | | | | | | | A | S | M | D | Y | 10 | Z |
| 1170 | A | R | Y | D | Y | | | | | | | | Y | W | M | D | Y | 10 | Z |
| 1524 | A | R | H | D | Y | | | | | | | | G | A | L | D | Y | 10 | Z |
| 1164 | A | R | G | V | P | H | | | | | | | G | Y | F | D | Y | 11 | P |
| 1168 | A | R | Y | F | P | H | | | | | | | Y | Y | F | D | Y | 11 | P |
| 1490 | A | R | Y | Y | P | H | | | | | | | H | Y | I | D | Y | 11 | P |
| 1542 | A | R | G | Y | P | H | | | | | | | H | Y | F | D | Y | 11 | P |
| 1514 | A | R | S | G | Y | S | N | | | | W | A | N | S | F | D | Y | 14 | |
| 1526 | A | R | Y | Y | F | H | D | Y | | | A | A | Y | S | L | D | Y | 15 | |
| 1520 | A | R | Y | Y | Y | S | H | G | Y | Y | V | Y | G | T | L | D | Y | 17 | |

### Example 9—Domain Mapping of OX40 Antibodies

The extracellular part of OX40 consists of four domains, each of which can be subdivided into two modules. Genes of OX40 human/mouse chimeras were synthesized using standard laboratory techniques. The different chimeras were designed by exchanging domains or modules of the human OX40 with corresponding mouse OX40. The chimeras were designed based on evaluation of the human and mouse sequences and 3D investigation of human OX40. The synthesized genes were assigned project specific ID numbers (see Table 10). The constructs were cloned into pcDNA3.1 vector (Invitrogen).

The mouse/human chimeras were transiently transfected into FreeStyle 293-F cells (Invitrogen), incubated 48 hours in FreeStyle 293 expression medium (Invitrogen) 37 C, 8% CO₂, 135 rpm. The transfected cells were incubated with human OX40 antibodies, human OX40L (hOX40L, RnD Systems), mouse OX40L (mOX40L, RnD Systems) and controls for 30 min 4° C. and then detected with a-huIgG-PE (Jackson Immunoresearch) 30 min 4° C. Cells were analyzed with FACS Verse (BD Biosciences). Binding to the different chimeric constructs were calculated as relative (mean fluorescence intensity) MFI compared to the binding of the isotype control. Results are shown in Table 11.

None of the human OX40 antibodies tested bind to murine OX40. Accordingly, if a given antibody does not bind to a particular chimera, this indicates that the antibody is specific for one of the domains which has been replaced with a murine domain in that chimera.

TABLE 10
Identity of chimeric constructs

| ID | Description of coding region |
|---|---|
| construct | of the chimeric DNA constructs |
| 1544 | Human OX40 with mouse domains 1A, 1B and |
| | 2A (aa 30-81) |
| 1545 | Human OX40 with mouse domains 1B, 2A and |
| | 2B (aa 43-107) |
| 1546 | Human OX40 with mouse domains 2A, 2B and |
| | module 3 (aa 66-126) |
| 1547 | Human OX40 with mouse domain 2B, module 3 |
| | and domain 4A (aa 83-141) |
| 1548 | Human OX40 with mouse module 3 and domains |
| | 4A and 4B (aa 108-167) |
| 1549 | Human OX40 with mouse domains 1A and 4B |
| | and region non-annotated extracellular region |
| | (aa 30-65 and aa 127-214) |
| 84 | Construct containing the full length OX40 sequence |
| 57 | Empty vector (negative control) |

At least four separate binding patterns were identified.

Pattern A:

Antibodies 1170/1171, 1524/1525, and 1526/1527 display a similar binding pattern and depend on residues in the same domains for binding. Amino acid residues critical for binding are likely located in module B in domain 2, and in module A of domain 2. The majority of the antibodies with CDRH3 family “Z” bind according to pattern A (1166/1167 being the exception), indicating that antibodies with this type of CDRH3 are predisposed to bind this epitope.

Pattern B:

Antibodies 1168/1135, 1542/1135, 1520/1135, 1490/1135, 1482/1483 and 1164/1135 display a similar binding pattern and depend mainly on residues located in Domain 3 for binding. All antibodies with CDRH3 family “P” binds with this pattern, demonstrating that the similarity in CDRH3 sequence reveals a common binding epitope.

Pattern C:

Antibody 1166/1167 has a unique binding pattern and likely depends on residues located in module A and module B in domain 2 for binding. However, both modules must be exchanged simultaneously to abolish binding, suggesting a structurally complex epitope.

Pattern D:

Antibody 1514/1515 displays a unique binding profile and likely depends mostly on amino acids located in module B in domain 2 for binding.

Reference antibody 72 binds according to pattern B. The binding pattern of the human OX40 ligand is similar to pattern C.

TABLE 11
Results from domain mapping experiment

| Chimeric OX40 | | 1490/ | 1170/ | 1524/ | 1526/ | 1482/ | 1514/ | 1164/ | 1168/ | 1520/ | 1542/ | 1166/ | polyclonal |
|---|---|---|---|---|---|---|---|---|---|---|---|---|---|
| construct | Antibody | 1135 | 1171 | 1525 | 1527 | 1483 | 1515 | 1135 | 1135 | 1135 | 1135 | 1167 | antibody |
| 1544 | | 4.2 | 2.0 | 1.5 | 1.1 | 8.4 | 11.6 | 14.0 | 13.2 | 9.4 | 7.1 | 14.2 | 50.9 |
| 1545 | | 28.9 | 1.0 | 0.9 | 1.3 | 44.4 | 1.2 | 37.3 | 46.6 | 32.6 | 40.2 | 1.0 | 58.6 |
| 1546 | | 1.0 | 0.8 | 0.9 | 0.9 | 0.9 | 0.9 | 0.9 | 0.9 | 0.9 | 0.9 | 0.8 | 30.0 |
| 1547 | | 1.1 | 2.3 | 1.0 | 1.0 | 1.0 | 1.1 | 1.0 | 1.0 | 0.9 | 1.0 | 15.2 | 43.9 |
| 1548 | | 1.1 | 20.3 | 15.4 | 12.7 | 1.1 | 17.2 | 1.0 | 1.0 | 1.1 | 1.2 | 15.8 | 31.7 |
| 1549 | | 5.9 | 12.1 | 11.2 | 8.5 | 8.7 | 10.6 | 14.1 | 15.1 | 8.5 | 13.0 | 11.3 | 26.3 |
| 84 | | 14.2 | 33.4 | 31.4 | 21.4 | 24.9 | 25.9 | 27.5 | 29.6 | 25.4 | 29.4 | 27.6 | 53.2 |
| 57 | | 1.0 | 1.0 | 1.0 | 1.0 | 1.0 | 1.1 | 1.0 | 0.9 | 1.0 | 1.1 | 1.0 | 1.2 |

### Example 10—Cross Reactivity with Macaca mulatta

The extracellular part of OX40 from Macaca mulatta was fused to the transmembrane and intracellular part of hCD40 and cloned into pcDNA3.0. The vector was subsequently stably transfected into HEK cells (macOX40-HEK).

Expression of OX40 was confirmed by incubating with commercial OX40 antibody (huOX40, BD Biosciences) for 30 min at 4° C. and then detected with a-huIgG-PE (Jackson Immunoresearch) 30 min 4° C. For the assay, the transfected cells were incubated with the test antibodies and controls for 30 min at 4° C. and then detected with a-huIgG-PE (Jackson Immunoresearch) 30 min 4° C. Cells were analyzed by flow cytometry with FACS Verse (BD Biosciences).

As shown in Table 12 below, tested antibodies bind to Macaca mulatta OX40 with EC50 values comparable to those achieved for human OX40, suggesting that Macaca mulatta will be suitable for use in toxicology studies.

Macaca mulatta is genetically very similar to Macaca fascicularis (cynomolgus monkey) making it very likely that cynomolgus monkey is also a suitable species for toxicology studies.

TABLE 12
Binding to human and monkey OX40 (95% confidence intervals)

| OX40 | Binding to M mulatta | Binding to human |
|---|---|---|
| antibody | OX40, EC50 (μg/ml) | OX40, EC50 (μg/ml) |
| 1166/1167 | 0.1595 to 0.2425 | 0.1415 to 0.2834 |
| 1168/1135 | 0.09054 to 0.1939 | 0.06360 to 0.1308 |
| 1482/1483 | 0.1565 to 0.3120 | 0.08196 to 0.1822 |
| 1520/1135 | 0.1632 to 0.3587 | 0.09247 to 0.2749 |
| 1526/1527 | 0.2921 to 0.5888 | 0.1715 to 0.4292 |
| 1542/1135 | 0.7221 to 1.414 | 0.3223 to 0.5525 |

### Example 11—Agonistic Activity in a Human T Cell Assay

Human T cells were obtained by negative T cell selection kit from Miltenyi from PBMC from leucocyte filters obtained from the blood bank (Lund University Hospital). The OX40 antibodies were coated to the surface of a 96 well culture plate (Corning Costar U-shaped plates (#3799) and cultured with a combination of immobilized anti-CD3 antibody (UCHT1), at 3 μg/ml, and soluble anti-CD28 antibody (CD28.2), at 5 μg/ml. Anti-CD3 was pre-coated overnight at 4° C. On the following day, after one wash with PBS, the OX40 antibodies were coated 1-2 h at 37° C. After 72 h incubation in a moisture chamber at 37° C., 5% CO₂ the IL-2 levels in the supernatant were measured.

The ability of the antibodies to stimulate human T cells to produce IL-2 was compared with the reference antibody 74 and the relative activity is displayed in FIG. 8. The majority of the antibodies provided T cell activation levels that were comparable with the reference antibody. A number of antibodies provided higher levels of T cell activation.

### Example 12—Production of OX40-5T4 Bispecific Antibodies

OX40-5T4 bispecific antibodies, based on four OX40 and two 5T4 antibodies were cloned as bsAb with the 5T4 binding moieties cloned as a scFv and fused to the C-terminus of the heavy chain of the OX40 IgG. Bispecific antibodies were produced by transient transfection of Freestyle293 cells (Thermo Fischer) and purified by Protein A chromatography. The OX40-5T4 bispecific antibodies are listed in Table 13.

TABLE 13
OX40-5T4 bispecific antibodies

| | | OX40 parental clone | | 5T4 parental clone | |
|---|---|---|---|---|---|
| | Protein name | VH | VL | VH | VL |
| 1 | 1170/1167-1210/1211 | 1170 | 1167 | 1210 | 1211 |
| 2 | 1482/1483-1210/1211 | 1482 | 1483 | 1210 | 1211 |
| 3 | 1170/1167-1208/1135 | 1170 | 1167 | 1208 | 1135 |
| 4 | 1482/1483-1208/1135 | 1482 | 1483 | 1208 | 1135 |
| 5 | 1166/1167-1210/1211 | 1166 | 1167 | 1210 | 1211 |
| 6 | 1170/1171-1210/1211 | 1170 | 1171 | 1210 | 1211 |
| 7 | 1166/1167-1208/1135 | 1166 | 1167 | 1208 | 1135 |
| 8 | 1170/1171-1208/1135 | 1170 | 1171 | 1208 | 1135 |

### Example 13—Binding to Human OX40 and 5T4 by OX40-5T4 Bispecific Antibodies Measured by ELISA

Materials and Methods

A dual ELISA was used to detect simultaneous binding of the two targets by the bispecific antibodies. ELISA plates were coated with OX40-Fc (R&D systems, #3388-OX, 0.5 μg/ml) over night at 4° C. followed by blocking with PBST+2% bovine serum albumin (BSA) for 1 h at room temperature (RT). Bispecific antibodies and monospecific controls: 1166/1167 (targeting OX40) and 1210/1211 (targeting 5T4), were added in duplicates in serial dilutions to the plates and incubated for 1 h at RT. Biotinylated 5T4-Fc (0.5 μg/ml), followed by HRP-labelled streptavidin (Pierce #21126) were used for detection. Super signal ELISA Pico Chemiluminescent Substrate (Thermo Scientific, #37069) was used as substrate and luminescence was measured using FLUOstar Optima.

Results and Conclusions

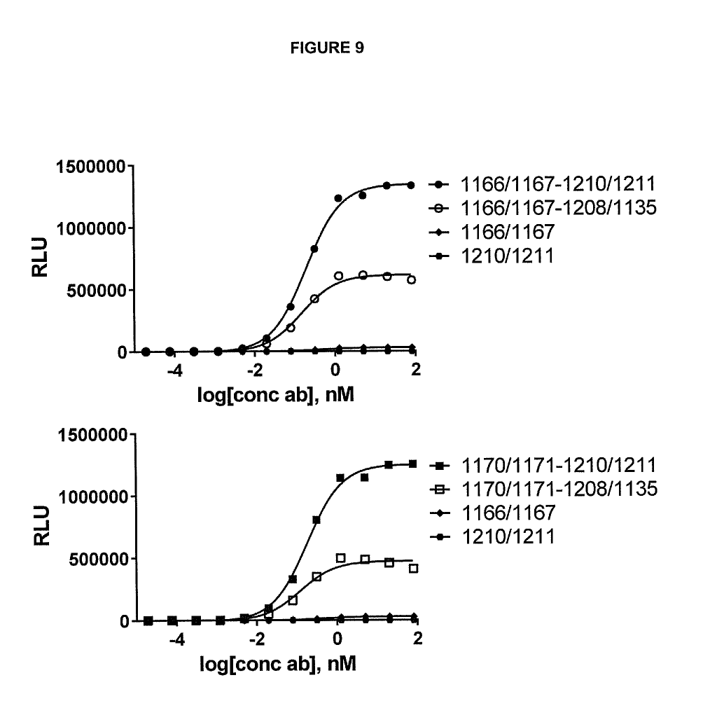

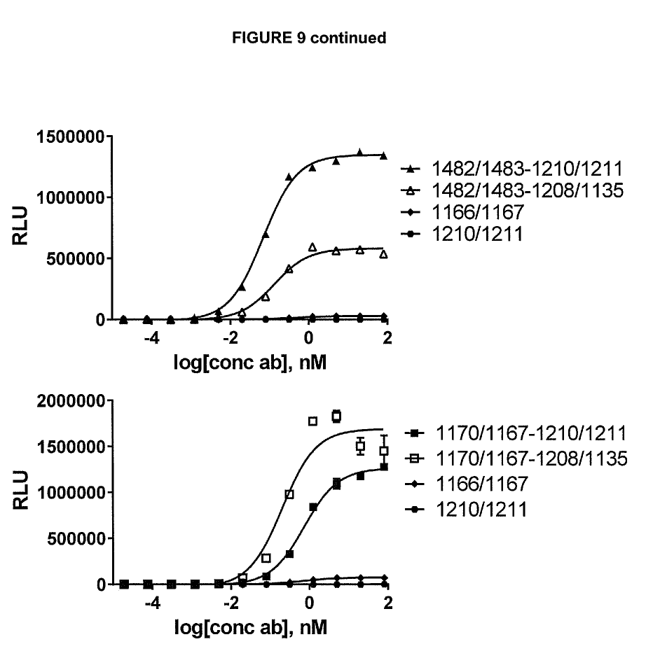

Simultaneous binding to both targets was shown for the bispecific antibodies as shown in FIG. 9 whereas the two negative control monospecific antibodies 1166/1167 and 1210/1211 did not bind to both targets.

In Table 14, the EC50 values of the eight bispecific antibodies are summarized.

TABLE 14
EC50 of the bispecific antibodies in dual ELISA (nM)

| Bispecific | 1166/1167- | 1170/1171- | 1482/1483- | 1166/1167- | 1170/1171- | 1482/1483- | 1170/1167- | 1170/1167- |
|---|---|---|---|---|---|---|---|---|
| antibody | 1210/1211 | 1210/1211 | 1210/1211 | 1208/1135 | 1208/1135 | 1208/1135 | 1210/1211 | 1208/1135 |
| EC50 | 0.19 | 0.18 | 0.069 | 0.15 | 0.12 | 0.13 | 0.74 | 0.20 |
| (nM) | | | | | | | | |

### Example 14—Functional Activity of OX40-5T4 Bispecific Antibodies in a Human CD4+ T Cells Assay

Materials and Methods

The functional activity of OX40-5T4 bispecific antibodies was evaluated in a CD4 T cell assay, where cells were cultured in microtiter plates coated with 5T4-Fc and CD3 antibody. Peripheral blood mononuclear cells were isolated by density gradient centrifugation using Ficoll-Paque (GE Healthcare #17-1440-02) from leucocytes concentrates obtained from healthy donors (Clinical Immunology and Transfusion Medicine, Labmedicin Region Skåne, Lund Sweden). CD4⁺T cells were enriched by negative selection using the CD4⁺ T cell isolation kit (Miltenyi 130-096-533). 96-well culture plates (Thermo Scientific Nunc #268200) were coated overnight at 4° C. with anti-CD3 antibody (UCHT-1, 1 μg/ml), followed by coating of 5T4-Fc (1.25 μg/ml) 2 h at 37° C. Wells not coated with 5T4 were used as negative control.

OX40-5T4 bispecific antibodies were diluted in RPMI containing 10% FCS and 10 mM Hepes and added in duplicates in a serial dilution to the wells with or without coated 5T4 30 min prior to the addition of CD4 T cells. Negative controls, comprising the corresponding monospecific antibodies or isotype control antibody were used at equimolar concentrations. Following 72 h of incubation at 37° C., 5% C002, supernatants were harvested and IL-2 levels measured using BD OptEIA Human IL-2 ELISA set (BD Biosciences, #55190). SuperSignal ELISA Pico Chemiluminescent Substrate (Thermo Scientific, #37069) was used as substrate and luminescence was measured using FLUOstar Optima. Data is presented as mean±SEM.

Results and Conclusions

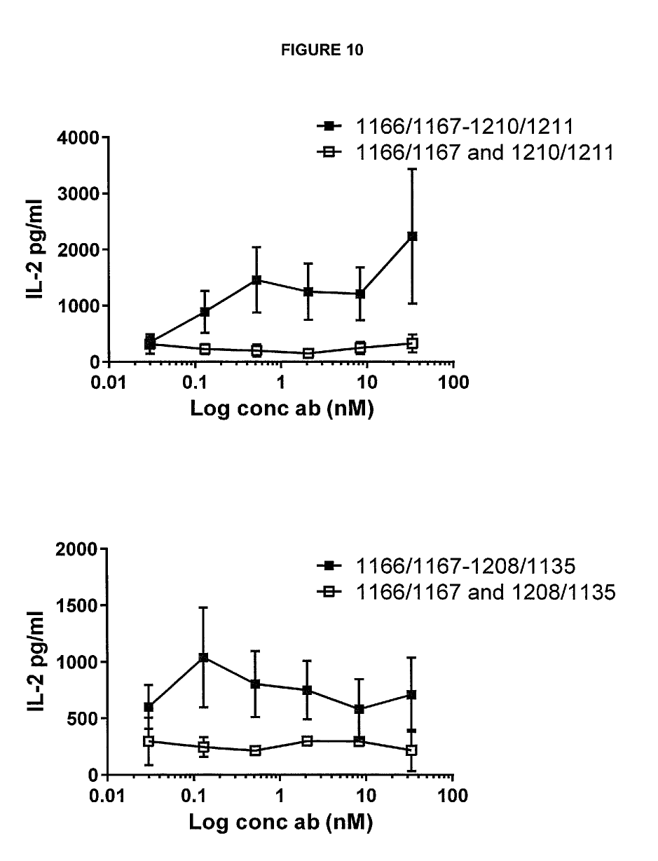

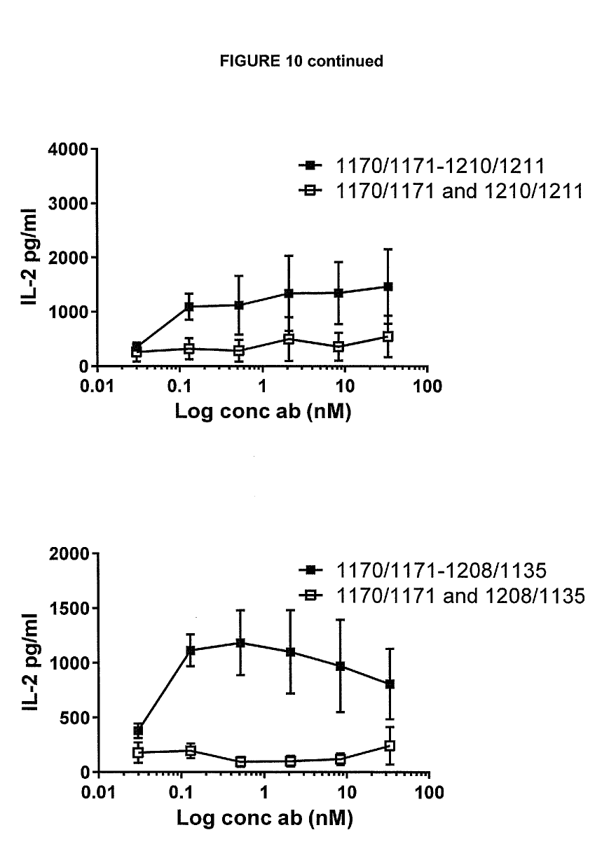

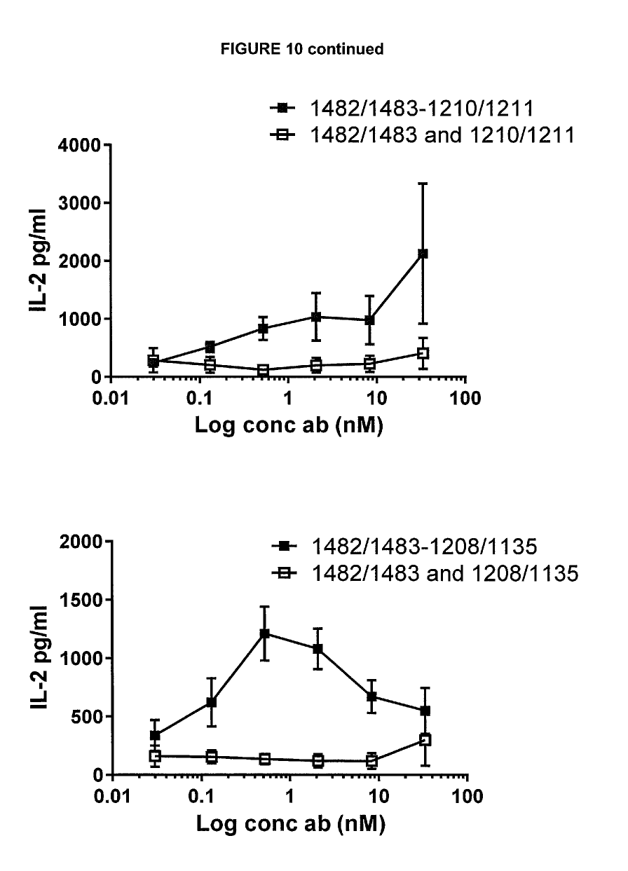

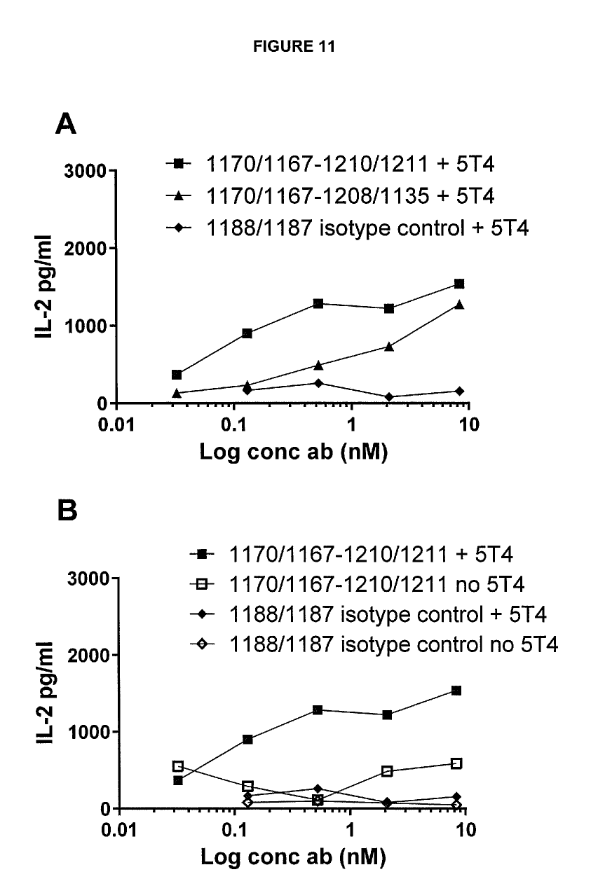

As shown in FIG. 10 and in FIG. 11 the OX40-5T4 bispecific antibodies specifically activate CD4+ T cells in a dose-dependent manner, whereas no activation is obtained upon incubation with the combination of monospecific antibodies, or with isotype control antibody.

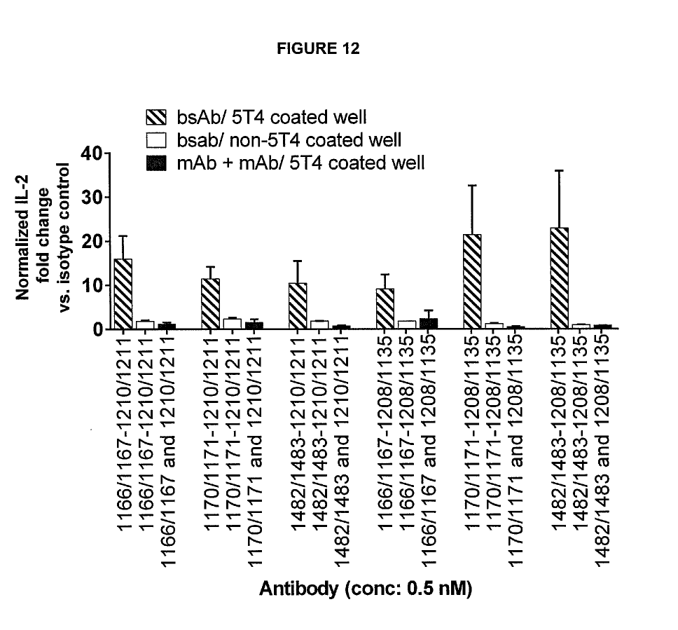

To reduce the variation between different plates, different background levels with or without 5T4 coating and different donors, samples were normalized to the sample of the negative control (the isotype control 1188/1187) on the corresponding ELISA plate (with same coating, from same donor), and is presented in FIG. 12 as fold change in IL-2 versus the isotype control.

### Example 15—TAA-OX40 bsAbs Dual-Binding ELISA

Materials and Methods

Bispecific antibodies against three tumor associated antigen, EpCam, HER2 and EGFR were generated. A scFv (1168/1135) binding to OX40 was fused to the C-terminal end of three different IgG antibodies with the sequences corresponding to the binding domains of an EpCAM binding antibody, an EGFR binding antibody and a HER2 binding antibody. TAA-OX40 bsAbs were produced by transient transfection of Expi293™ (Thermo Fischer Scientific) and purified by Protein A chromatography. Table 15 shows a summary of the generated TAA-OX40 bsAbs,

Bispecific binding to both targets, OX40 and TAA, was evaluated using a standard ELISA protocol. Plates (#655074, Greiner Bio-One GmbH, Germany) were pre-coated with 0.5 μg/ml TAA, either hEGFR-His, (SinoBiological), hEpCAM-Fc, (SinoBiological) or HER2-His, (SinoBiological) overnight. The different TAA-OX40 bsAb were diluted from 20 μg/ml in 1:4 dilutions and added in duplicates of 50 μl to each well. Biotinylated human OX40, OX40-bio (Ancell, #513-030, at 0.5 μg/ml), was used to detect bound TAA-bsAb antibody and the binding was detected with Streptavidin-HRP (Pierce #21126). The ELISA was developed with SuperSignal ELISA PICO Chemiluminescent substrate (Thermo Scientific Pierce, Rockford, Ill. USA) during 2-10 minutes and read in an automated microplate based multi-detection reader (FLUOstar OPTIMA, Netherlands).

TABLE 15
Summary of generated TAA-OX40 bsAbs

| mAb | scFv | TAA-OX40 bsAb name | |
|---|---|---|---|
| EpCam (2414) | OX40 (1168/1135) | 2414-1168 | 15 |
| EGFR (2424) | OX40 (1168/1135) | 2424-1168 | |
| HER2 (2078) | OX40 (1168/1135) | 2078-1168 | |

Results and Conclusions

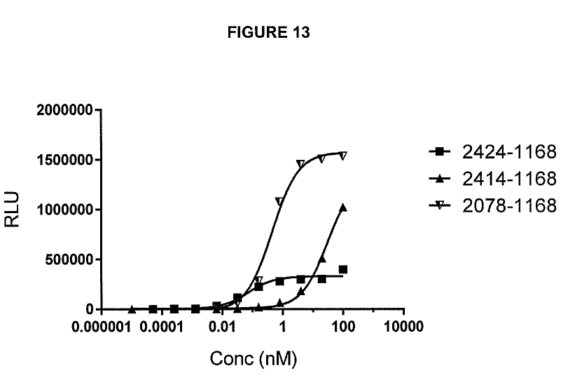

EGFR-OX40 and Her2-OX40 bsAbs binds to both targets in a dual ELISA (as shown in FIG. 13) with EC50 values in the low nM range. The EC50 for EpCam-OX40 (2414-1168) was not calculated.

### Example 16—Functional Activity of TAA-OX40 Bispecific Antibodies on Human CD4+ T Cells Cultured in TAA Coated Plates

Materials and Methods

The functional activity of the three TAA-OX40 bsAb binding to EpCAM, EGFR and HER2 was evaluated in a CD4 T cell assay, where cells were cultured in microtiter plates coated with CD3 antibody and either EGFR, EpCam or HER2. As negative control, parallel wells were coated with only CD3 antibody (no TAA). Peripheral blood mononuclear cells (MNC) were isolated by density gradient centrifugation using Ficoll-Paque (ρ 1.077 g/ml) (GE Healthcare #17-1440-02) from leucocytes concentrate obtained from healthy donors (Clinical Immunology and Transfusion Medicine, Labmedicin Region Skåne, Lund Sweden). CD4⁺T cells were enriched by negative selection using the CD4⁺ T cell isolation kit (Miltenyi 130-096-533). Plates were coated overnight at 4° C. with 1 μg/ml αCD3, clone UTCH-1 (BD, #555329), washed and coated with 5 μg/ml TAA for 2 h at 37° C. After the TAA coating, plates were washed and blocked for a minimum of 30 minutes with RPMI (Gibco #61870010) containing 10% FCS (Heat inactivated, Gibco #10270-106 lot 41Q9248K) and 10 mM Hepes (Gibco #15630056).

TAA-OX40 bsAbs were diluted in RPMI containing 10% FCS and 10 mM Hepes and added to the plates 30 minutes before addition of CD4⁺ T cells (0.07×10⁶ cells/well). Assay plates were incubated for 68 h at 37° C., and culture supernatant harvested. IL2 levels in the supernatants were measured by ELISA (BD OptiEIA #555142).

Results and Conclusions

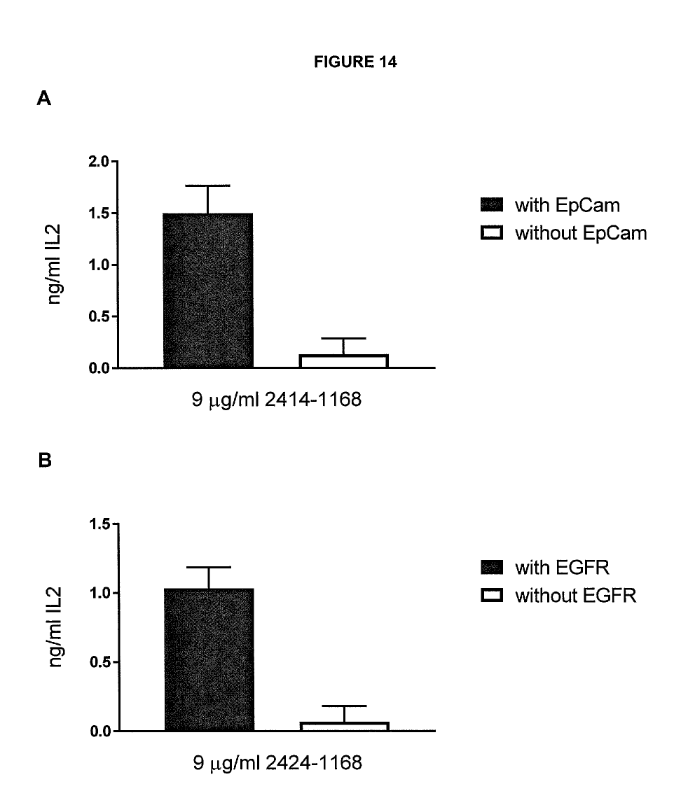

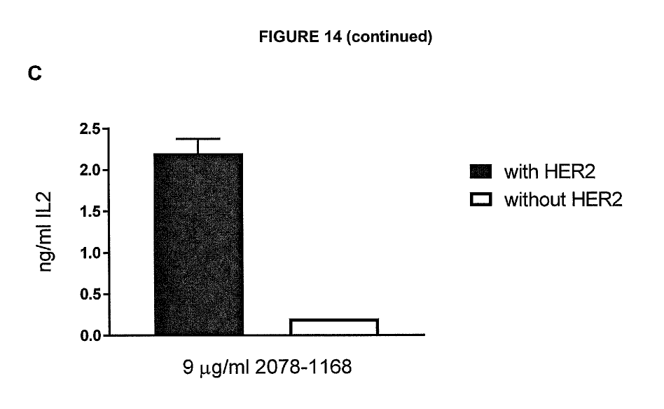

The functionality of the EpCAM-1168 (FIG. 14A), EGFR-1168 (FIG. 14B) and HER2-1168 (FIG. 14C) bsAbs was analysed in a CD4 T cell assay and it was concluded that the generated bsAbs induce TAA mediated OX40 activation in the presence of TAA and not in the absence of TAA (wells coated with only CD3 antibody). The results support a broader concept that cell surface expressed TAA/receptor could induce an OX40 mediated immune cell activation.

### REFERENCES

Altschul et al, 1990, J Mol Biol 215:403-10,
Altschul, 1993, J Mol Evol 36:290-300
Angov, 2011, Biotechnol. J. 6(6):650-659
Boerner et al., 1991. J. Immunol. 147:86-95,
Bruhns et al., 2009, Blood. 113(16):3716-25
Bruhns, 2012, Blood. 119(24):5640-9.
Caceci et al. Byte 9:340-362, 1984
Castro, F. V. et al. (2012) Leukemia. 26(7): 1487-1498.
Chan & Carter, 2016, Nature Reviews Immunology 10, 301-316
Cheever, M. A. et al. (2009). Clin. Cancer Res. 15, 5323-5337.
Cole et al., 1984. Mol. Cell. Biol. 62:109-120
Cole et al., 1985, In: Monoclonal antibodies and Cancer Therapy, Alan R. Liss, pp. 77
Cote et al., 1983. Proc. Natl. Acad. Sci. USA 80:2026-2030
Damelin, M. et al. (2011) Cancer Res 71, 4236-4246.
Devereux et al. 1984, Nucleic Acids Research 12:387-395
Elkord, E. et al. (2009) Expert Rev Anticancer Ther 9, 1705-1709.
Gebauer & Skerra, 2009, Curr Opin Chem Biol 13(3): 245-255
Henikoff & Henikoff, 1992, Proc. Natl. Acad. Sci. USA 89:10915-10919
Hezareh et al., 2001, J Virol, 75(24):12161-8
Hinton et al., 2004, J Biol Chem. 279(8):6213-6
Hole, N. and Stern, P. L. (1988) Br. J Cancer 57, 239-246.
Hogarth and Pietersz, 2012, Nature Reviews Drug Discovery 11, 311-331
Hoogenboom & Winter, 1991, J. Mol. Biol. 227:381
Jones et al., 1986. Nature 321:522-525
Ju and Jung, 2014, Curr Opin Biotechnol. 30:128-39.
Karlin & Altschul, 1993, Proc. Natl. Acad. Sci. USA 90:5873-5787
Kiefer, J. D. and Neri, D. (2016) Immunol. Rev. 270, 178-192.
Kohler et al., 1975. Nature 256:4950497
Kozbor et al., 1985. J. Immunol. Methods 81:31-42
Leabman et al. 2013, mAbs, 5:6, 896-903
Lines et al. 2014 Cancer Immunol Res. 2(6): 510-517.
Lund, K. et al 2014. mAbs 6, 1038-1050.
Marks et al., 1991, J. Mol. Biol. 222:581
Meziere et al. (1997) J. Immunol. 159, 3230-3237
Oganesyan et al. 2008 Acta Crystallogr D Biol Crystallogr. 64(Pt 6): 700-704.
Oganesyan et al. 2008 Mol Immunol. 45(7):1872-82.
Orlandi. et al, 1989. Proc. Natl. Acad. Sci. U.S.A. 86:3833-3837
Patriarca, C. et al. 2012. Cancer Treat Rev 38, 68-75.
Presta, 1992, Curr. Op. Struct. Biol. 2:593-596
Pule et al., 2003, Cytotherapy 5(3):211-26
Riechmann et al., 1988, Nature 332:323-329
Sazinsky et al., 2008 Proc. Natl. Acad. Sci. U.S.A 105(51) 20167-20172;
Schnell et al. 2013. Biochim Biophys Acta 1828, 1989-2001.
Shields et al., 2001, J Biol Chem. 276(9):6591-604
Southall et al. (1990) Br. J Cancer 61, 89-95.
Southgate et al. (2010) PLoS. ONE. 5, e9982.
Stewart et al. 2014 Journal for ImmunoTherapy of Cancer 2:29
Strohl, 2009, Curr Opin Biotechnol 20(6):685-91
Thompson et al., 1994, Nucleic Acids Res. 22(22):4673-80
Thursell et al., 1983, Biochem. Biophys. Res. Comm. 111:166
Vaccaro et al., 2005, Nat. Biotechnol. 23(10):1283-8
Veber et al., 1978, Proc. Natl. Acad. Sci. USA 75:2636
Verhoeyen et al., 1988, Science 239:1534-15361
Vidarsson et al. 2014, Front Immunol. 5:520
Wang et al. 2015 Front Immunol; 6: 368.
Whitlow et al. 1993 Protein Eng. 6(8):989-95.
Winter et al., 1991, Nature 349:293-299,
Wong & Lohman, Proc. Natl. Acad. Sci. USA 90, 5428-5432, 1993
Woods et al., 2002, Biochem J 366(1):353-365
Yao et al. 2013, Int J Cancer December 15; 133(12):2925-33
Yarden, Y. 2001. Eur J Cancer 37 Suppl 4, S3-8.
Yarden, Y., and Sliwkowski, M. X. 2001. Nat Rev Mol Cell Biol 2, 127-137.

### PATENT REFERENCES

US 2010/0021483
U.S. Pat. No. 4,816,567
US 2012/0251531
U.S. Pat. No. 4,235,871
U.S. Pat. No. 5,851,451
EP 0 213 303
U.S. Pat. No. 7,557,190
U.S. Pat. No. 7,060,808

1. A bispecific polypeptide comprising a first binding domain, designated B1, which is capable of binding specifically to OX40, and a second binding domain, designated B2, which is capable of specifically binding to a tumour cell-associated antigen.

2. A polypeptide according to claim 1, wherein the first and/or second binding domains are/is selected from the group consisting of antibodies and antigen-binding fragments thereof.

3. A polypeptide according to claim 2 wherein the antigen-binding fragment is selected from the group consisting of: Fv fragments (such as a single chain Fv fragment, or a disulphide-bonded Fv fragment), Fab-like fragments (such as a Fab fragment; a Fab′ fragment or a F(ab)₂ fragment) and domain antibodies.

4. A polypeptide according to any one of the preceding claims wherein the polypeptide is a bispecific antibody.

5. A polypeptide according to claim 4 wherein:
(a) binding domain B1 and/or binding domain B2 is an intact IgG antibody;
(b) binding domain B1 and/or binding domain B2 is an Fv fragment;
(c) binding domain B1 and/or binding domain B2 is a Fab fragment; and/or
(d) binding domain B1 and/or binding domain B2 is a single domain antibody.

6. A polypeptide according to claim 4 or 5 wherein the bispecific antibody comprises a human Fc region or a variant of a said region, where the region is an IgG1, IgG2, IgG3 or IgG4 region, preferably an IgG1 or IgG4 region.

7. A polypeptide according to claim 6 wherein the Fc exhibits no or very low affinity for FcgR.

8. A polypeptide according to claim 6 or 7 wherein the Fc region is a variant of a human IgG1 Fc region comprising a mutation at one or more of the following positions:
L234, L235, P239, D265, N297 and/or P329.

9. A polypeptide according to claim 8 wherein alanine is present at the mutated positions(s).

10. A polypeptide according to claim 9 wherein the Fc region is a variant of a human IgG1 Fc region comprising the double mutations L234A and L235A.

11. A polypeptide according to any one of claims 4 to 10 wherein the bispecific antibody is selected from the groups consisting of:
(a) bivalent bispecific antibodies, such as IgG-scFv bispecific antibodies (for example, wherein B1 is an intact IgG and B2 is an scFv attached to B1 at the N-terminus of a light chain and/or at the C-terminus of a light chain and/or at the N-terminus of a heavy chain and/or at the C-terminus of a heavy chain of the IgG, or vice versa);
(b) monovalent bispecific antibodies, such as a DuoBody® or a ‘knob-in-hole’ bispecific antibody (for example, an scFv-KIH, scFv-KIH^{r}, a BiTE-KIH or a BiTE-KIH^{r};
(c) scFv₂-Fc bispecific antibodies (for example, ADAPTIR™ bispecific antibodies);
(d) BiTE/scFv₂ bispecific antibodies;
(e) DVD-Ig bispecific antibodies;
(f) DART-based bispecific antibodies (for example, DART₂-Fc, DART₂-Fc or DART);
(g) DNL-Fab₃ bispecific antibodies; and
(h) scFv-HSA-scFv bispecific antibodies.

12. A polypeptide according to claim 11 wherein the bispecific antibody is an IgG-scFv bispecific antibody.

13. A polypeptide according to any one of the preceding claims wherein binding domain B1 and binding domain B2 are fused directly to each other.

14. A polypeptide according to any one of claims 1 to 12 wherein binding domain B1 and binding domain B2 are joined via a polypeptide linker.

15. A polypeptide according to claim 14 wherein the linker is selected from the group consisting of the amino acid sequence SGGGGSGGGGS (SEQ ID NO: 104), SGGGGSGGGGSAP (SEQ ID NO: 105), NFSQP (SEQ ID NO: 106), KRTVA (SEQ ID NO: 107), GGGSGGGG (SEQ ID NO: 108), GGGGSGGGGS (SEQ ID NO: 109), GGGGSGGGGSGGGGS (SEQ ID NO: 110), GSTSGSGKPGSGEGSTKG (SEQ ID NO: 116), THTCPPCPEPKSSDK (SEQ ID NO: 117), GGGS (SEQ ID NO: 118), EAAKEAAKGGGGS (SEQ ID NO: 119), EAAKEAAK (SEQ ID NO: 120), or (SG)m, where m=1 to 7.

16. A polypeptide according to any one of the preceding claims, wherein the polypeptide is incapable of inducing antibody dependent cell cytotoxicity (ADCC), antibody-dependent cellular phagocytosis (ADCP) and/or complement-dependent cytotoxicity (CDC).

17. A polypeptide according to any one of the preceding claims, wherein the polypeptide is capable of inducing tumour immunity.

18. A polypeptide according to any one of the preceding claims, wherein the polypeptide is capable of inducing:
(a) activation of cytotoxic T cells, i.e. CD8+ T cells;
(b) activation of helper T cells, i.e. CD4⁺ T cells;
(c) activation of dendritic cells; and/or
(d) activation of natural killer cells; and/or
(e) reprogramming of Tregs into effector T cells.

19. A polypeptide according to any one of the preceding claims wherein binding domain B1 binds to human OX40 with a K_{D} of less than 50×10⁻¹⁰M or less than 25×10⁻¹⁰M, more preferably less than 10, 9, 8, 7, or 6×10⁻¹⁰M, most preferably less than 5×10⁻¹⁰M.

20. A polypeptide according to any one of the preceding claims, wherein B1 exhibits at least one of the following functional characteristics when present independently of B2:
I. binding to human OX40 with a K_{D} value which is less than 10×10⁻¹⁰M, more preferably less than 5×10⁻¹⁰M;
II. does not bind to murine OX40; and
III. does not bind to other human TNFR superfamily members, for example human CD137 or CD40

21. A polypeptide according to any one of the preceding claims, wherein B1 comprises any one, two, three, four, five or all six features independently selected from the following:
(a) a heavy chain CDR1 sequence which is 8 amino acids in length and comprises the consensus sequence: “G, F, T, F, G/Y/S, G/Y/S, Y/S, Y/S/A”;
(b) a heavy chain CDR2 sequence which is 8 amino acids in length and comprises the consensus sequence: “I, G/Y/S/T, G/S/Y, S/Y, G/S/Y, G/S/Y, G/S/Y, T”;
(c) a heavy chain CDR3 sequence which is 9 to 17 amino acids in length and which comprises the consensus sequence of: “A, R, G/Y/S/H, G/Y/F/V/D, G/Y/P/F, -/H/S, -/N/D/H, -/Y/G, -/Y, -/Y, -/W/A/V, -/A/Y, -/D/A/Y/G/H/N, Y/S/W/A/T, L/M/I/F, D, Y”
(d) a light chain CDR1 sequence which consists of the sequence: “Q, S, I, S, S, Y”;
(e) a light chain CDR2 sequence which consists of the sequence: “A, A, S”;
(f) a light chain CDR3 sequence which is 8 to 10 amino acids in length and comprises the consensus sequence: “Q,Q, S/Y/G, -/Y/H/G, -/S/Y/G/D, S/Y/G/D, S/Y/G/T, P/L, Y/S/H/L/F, T”;
wherein the heavy chain CDR3 sequence of (c) is preferably a sequence of 10 amino acids in length which comprises the consensus sequence “A, R, Y/H, D, Y, A/Y/G, S/W/A, M/L, D, Y” or a CDR3 sequence of 11 amino acids in length which comprises the consensus sequence “A, R, G/Y, V/F/Y, P, H, G/Y/H, Y, F/I, D, Y”; and
the light chain CDR3 sequence of (f) preferably consists of the sequence “Q, Q, S, Y, S, T, P, Y, T”.

22. A polypeptide according to any one of the preceding claims, wherein B1 comprises all three heavy chain CDR sequences of a VH sequence as shown in Table C(1) and/or all three light chain CDR sequences of a VL sequence as shown in Table C(2), or wherein B1 comprises a heavy chain VH sequence and/or a light chain VL sequence as shown in Table D.

23. A polypeptide according to any one of the preceding claims wherein binding domain B1 comprises:
(a) the three CDRs of the heavy chain and/or the three CDRs of the light chain of antibody 1166/1167 (SEQ ID NOs: 32, 40 and 49 and/or SEQ ID NOs: 26, 27 and 60);
(b) the three CDRs of the heavy chain and/or the three CDRs of the light chain of antibody 1170/1171 (SEQ ID NOs: 32, 41 and 50 and/or SEQ ID NOs: 26, 27 and 61);
(c) the three CDRs of the heavy chain and/or the three CDRs of the light chain of antibody 1164/1135 (SEQ ID NOs: 33, 42 and 51 and/or SEQ ID NOs: 26, 27 and 62);
(d) the three CDRs of the heavy chain and/or the three CDRs of the light chain of antibody 1168/1135 (SEQ ID NOs: 34, 43 and 52 and/or SEQ ID NOs: 26, 27 and 62);
(e) the three CDRs of the heavy chain and/or the three CDRs of the light chain of antibody 1482/1483 (SEQ ID NOs: 35, 44 and 53 and/or SEQ ID NOs: 26, 27 and 63);
(f) the three CDRs of the heavy chain and/or the three CDRs of the light chain of antibody 1490/1135 (SEQ ID NOs: 35, 43 and 54 and/or SEQ ID NOs: 26, 27 and 62);
(g) the three CDRs of the heavy chain and/or the three CDRs of the light chain of antibody 1514/1515 (SEQ ID NOs: 36, 45 and 55 and/or SEQ ID NOs: 26, 27 and 64);
(h) the three CDRs of the heavy chain and/or the three CDRs of the light chain of antibody 1520/1135 (SEQ ID NOs 35, 40 and 56 and/or SEQ ID NOs: 26, 27 and 62);
(i) the three CDRs of the heavy chain and/or the three CDRs of the light chain of antibody 1524/1525 (SEQ ID NOs: 37, 46 and 57 and/or SEQ ID NOs: 26, 27 and 65);
(j) the three CDRs of the heavy chain and/or the three CDRs of the light chain of antibody 1526/1527 (SEQ ID NOs: 38, 47 and 58 and/or SEQ ID NOs: 26, 27 and 66);
(k) the three CDRs of the heavy chain and/or the three CDRs of the light chain of antibody 1542/1135 (SEQ ID NOs: 39, 48 and 59 and/or SEQ ID NOs: 26, 27 and 62); or
(l) the three CDRs of the heavy chain and/or the three CDRs of the light chain of antibody 1170/1167 (SEQ ID NOs: 32, 41 and 50 and/or SEQ ID NOs: 26, 27 and 60).

24. A polypeptide according to any one of the preceding claims wherein binding domain B1 comprises:
(a) the heavy chain variable region and/or the light chain variable region of antibody 1166/1167 (SEQ ID NO: 69 and/or SEQ ID NO: 67);
(b) the heavy chain variable region and/or the light chain variable region of antibody 1170/1171 (SEQ ID NO: 73 and/or SEQ ID NO: 71);
(c) the heavy chain variable region and/or the light chain variable region of antibody 1164/1135 (SEQ ID NO: 77 and/or SEQ ID NO: 75);
(d) the heavy chain variable region and/or the light chain variable region of antibody 1168/1135 (SEQ ID NO: 79 and/or SEQ ID NO: 75);
(e) the heavy chain variable region and/or the light chain variable region of antibody 1482/1483 (SEQ ID NO: 83 and/or SEQ ID NO: 81);
(f) the heavy chain variable region and/or the light chain variable region of antibody 1490/1135 (SEQ ID NO: 85 and/or SEQ ID NO: 75);
(g) the heavy chain variable region and/or the light chain variable region of antibody 1514/1515 (SEQ ID NO: 89 and/or SEQ ID NO: 87);
(h) the heavy chain variable region and/or the light chain variable region of antibody 1520/1135 (SEQ ID NO: 91 and/or SEQ ID NO: 75);
(i) the heavy chain variable region and/or the light chain variable region of antibody 1524/1525 (SEQ ID NO: 95 and/or SEQ ID NO: 93);
(j) the heavy chain variable region and/or the light chain variable region of antibody 1526/1527 (SEQ ID NO: 99 and/or SEQ ID NO: 97);
(k) the heavy chain variable region and/or the light chain variable region of antibody 1542/1135 (SEQ ID NO: 101 and/or SEQ ID NO: 75);
(l) the heavy chain variable region and/or the light chain variable region of antibody 1170/1167 (SEQ ID NO: 73 and/or SEQ ID NO: 67); or
(m) variants of said heavy chain variable regions and/or said heavy chain variable regions having at least 90% sequence identity thereto.

25. A polypeptide according to any one of the preceding claims wherein binding domain B1 comprises:
(a) the light chain and/or the heavy chain of antibody 1166/1167;
(b) the light chain and/or the heavy chain of antibody 1170/1171;
(c) the light chain and/or the heavy chain of antibody 1164/1135;
(d) the light chain and/or the heavy chain of antibody 1168/1135;
(e) the light chain and/or the heavy chain of antibody 1482/1483;
(f) the light chain and/or the heavy chain of antibody 1490/1135;
(g) the light chain and/or the heavy chain of antibody 1514/1515;
(h) the light chain and/or the heavy chain of antibody 1520/1135;
(i) the light chain and/or the heavy chain of antibody 1524/1525;
(j) the light chain and/or the heavy chain of antibody 1526/1527;
(k) the light chain and/or the heavy chain of antibody 1542/1135; or
(l) the light chain and/or the heavy chain of antibody 1170/1167.

26. A polypeptide according to any one of the preceding claims wherein binding domain B1 comprises the heavy chain variable region and/or the light chain variable region of antibody 1170/1171 (SEQ ID NO: 73 and/or SEQ ID NO: 71)

27. A polypeptide according to any one of the preceding claims wherein binding domain B1 comprises the heavy chain variable region and/or the light chain variable region of antibody 1526/1527 (SEQ ID NO: 99 and/or SEQ ID NO: 97).

28. A polypeptide according to any one of the preceding claims wherein binding domain B1 comprises the heavy chain variable region and/or the light chain variable region of antibody 1168/1135 (SEQ ID NO: 79 and/or SEQ ID NO: 75).

29. A polypeptide according to any one of the preceding claims wherein binding domain B2 binds to a tumour cell-associated antigen selected from the group consisting of:
(a) products of mutated oncogenes and tumour suppressor genes;
(b) overexpressed or aberrantly expressed cellular proteins;
(c) tumour antigens produced by oncogenic viruses;
(d) oncofetal antigens;
(e) altered cell surface glycolipids and glycoproteins;
(f) cell type-specific differentiation antigens;
(g) hypoxia-induced antigens;
(h) tumour peptides presented by MHC class I;
(i) epithelial tumour antigens;
(j) haematological tumour-associated antigens;
(k) cancer testis antigens; and
(l) melanoma antigens.

30. A polypeptide according to any one of the preceding claims wherein the tumour cell-associated antigen is selected from the group consisting of 5T4, CD20, CD19, MUC-1, carcinoembryonic antigen (CEA), CA-125, CO17-1A, EpCAM, HER2, EphA2, EphA3, DR5, FAP, OGD2, VEGFR, Her3 and EGFR

31. A polypeptide according to any one of the preceding claims wherein the tumour cell-associated antigen is an oncofetal antigen.

32. A polypeptide according to any one of the preceding claims wherein the tumour cell-associated antigen is 5T4.

33. A polypeptide according to claim 30, wherein the tumour cell-associated antigen is selected from the group consisting of EGFR, EpCAM and HER2.

34. A polypeptide according to any one of the preceding claims wherein the tumour cell is a solid tumour cell.

35. A polypeptide according to claim 34 wherein the solid tumour is selected from the groups consisting of renal cell carcinoma, colorectal cancer, lung cancer, prostate cancer, breast cancer, melanomas, bladder cancer, brain/CNS cancer, cervical cancer, oesophageal cancer, gastric cancer, head/neck cancer, kidney cancer, liver cancer, lymphomas, ovarian cancer, pancreatic cancer and sarcomas.

36. A polypeptide according to any one of the preceding claims wherein binding domain B2 binds to the tumour cell-associated antigen with a K_{D} of less than 10×10⁻⁹M, for example less than 4×10⁻⁹M or less than 1.2×10⁻⁹M.

37. A polypeptide according to any one of the preceding claims wherein binding domain B2 comprises:
(a) the three CDRs of the light chain and/or the three CDRs of the heavy chain of antibody 1206/1207 (SEQ ID NOs: 26, 27 and 28 and/or SEQ ID NOs: 17, 19 and 22);
(b) the three CDRs of the light chain and/or the three CDRs of the heavy chain of antibody 1208/1135 (SEQ ID NOs: 26, 27 and 29 and/or SEQ ID NOs: 18, 20 and 23);
(c) the three CDRs of the light chain and/or the three CDRs of the heavy chain of antibody 1210/1211 (SEQ ID NOs: 26, 27 and 30 and/or SEQ ID NOs: 18, 20 and 24); and
(d) the three CDRs of the light chain and/or the three CDRs of the heavy chain of antibody 1212/1213 (SEQ ID NOs: 26, 27 and 31 and/or SEQ ID NOs: 18, 21 and 25).

38. A polypeptide according to any one of the preceding claims wherein binding domain B2 comprises:
(a) the light chain variable region and/or the heavy chain variable region of antibody 1206/1207 (SEQ ID NO: 3 and/or SEQ ID NO: 1);
(b) the light chain variable region and/or the heavy chain variable region of antibody 1208/1135 (SEQ ID NO: 7 and/or SEQ ID NO: 5);
(c) the light chain variable region and/or the heavy chain variable region of antibody 1210/1211 (SEQ ID NO: 11 and/or SEQ ID NO: 9);
(d) the light chain variable region and/or the heavy chain variable region of antibody 1212/1213 (SEQ ID NO: 15 and/or SEQ ID NO: 13); or
(e) variants of said light chain variable regions and/or said heavy chain variable regions having at least 90% sequence identity thereto.

39. A polypeptide according to any one of the preceding claims wherein binding domain B2 comprises:
(a) the light chain and/or the heavy chain of antibody 1206/1207;
(b) the light chain and/or the heavy chain of antibody 1208/1135;
(c) the light chain and/or the heavy chain of antibody 1210/1211; or
(d) the light chain and/or the heavy chain of antibody 1212/1213.

40. A polypeptide according to any one of the preceding claims wherein binding domain B2 comprises the light chain variable region and the heavy chain variable region of antibody 1208/1135 (SEQ ID NO: 7 and SEQ ID NO: 5).

41. A polypeptide according to any one of claims 1 to 39 wherein binding domain B2 comprises the light chain variable region and the heavy chain variable region of antibody 1210/1211 (SEQ ID NO: 11 and SEQ ID NO: 9).

42. A polypeptide according to any one of the preceding claims wherein binding domain B1 is an IgG and binding domain B2 is an scFv.

43. A polypeptide according to any one of claims 1 to 40 wherein binding domain B1 is an scFv and binding domain B2 is an IgG.

44. A polypeptide according to any one of the preceding claims wherein:
(a) B1 comprises the three CDRs of the heavy chain and/or the three CDRs of the light chain of antibody 1170/1167 (SEQ ID NOs: 32, 41 and 50 and/or SEQ ID NOs: 26, 27 and 60) and B2 comprises the three CDRs of the heavy chain and/or the three CDRs of the light chain of antibody 1210/1211 (SEQ ID NOs: 18, 20 and 24 and/or SEQ ID NOs: 26, 27 and 30);
(b) B1 comprises the three CDRs of the heavy chain and/or the three CDRs of the light chain of antibody 1482/1483 (SEQ ID NOs: 35, 44 and 53 and/or SEQ ID NOs: 26, 27 and 63) and B2 comprises the three CDRs of the heavy chain and/or the three CDRs of the light chain of antibody 1210/1211 (SEQ ID NOs: 18, 20 and 24 and/or SEQ ID NOs: 26, 27 and 30);
(c) B1 comprises the three CDRs of the heavy chain and/or the three CDRs of the light chain of antibody 1170/1167 (SEQ ID NOs: 32, 41 and 50 and/or SEQ ID NOs: 26, 27 and 60) and B2 comprises the three CDRs of the heavy chain and/or the three CDRs of the light chain of antibody 1208/1135 (SEQ ID NOs: 18, 20 and 23 and/or SEQ ID NOs: 26, 27 and 29);
(d) B1 comprises the three CDRs of the heavy chain and/or the three CDRs of the light chain of antibody 1482/1483 (SEQ ID NOs: 35, 44 and 53 and/or SEQ ID NOs: 26, 27 and 63) and B2 comprises the three CDRs of the heavy chain and/or the three CDRs of the light chain of antibody 1208/1135 (SEQ ID NOs: 18, 20 and 23 and/or SEQ ID NOs: 26, 27 and 29);
(e) B1 comprises the three CDRs of the heavy chain and/or the three CDRs of the light chain of antibody 1166/1167 (SEQ ID NOs: 32, 40 and 49 and/or SEQ ID NOs: 26, 27 and 60) and B2 comprises the three CDRs of the heavy chain and/or the three CDRs of the light chain of antibody 1210/1211 (SEQ ID NOs: 18, 20 and 24 and/or SEQ ID NOs: 26, 27 and 30);
(f) B1 comprises the three CDRs of the heavy chain and/or the three CDRs of the light chain of antibody 1170/1171 (SEQ ID NOs: 32, 41 and 50 and/or SEQ ID NOs: 26, 27 and 61) and B2 comprises the three CDRs of the heavy chain and/or the three CDRs of the light chain of antibody 1210/1211 (SEQ ID NOs: 18, 20 and 24 and/or SEQ ID NOs: 26, 27 and 30);
(g) B1 comprises the three CDRs of the heavy chain and/or the three CDRs of the light chain of antibody 1166/1167 (SEQ ID NOs: 32, 40 and 49 and/or SEQ ID NOs: 26, 27 and 60) and B2 comprises the three CDRs of the heavy chain and/or the three CDRs of the light chain of antibody 1208/1135 (SEQ ID NOs: 18, 20 and 23 and/or SEQ ID NOs: 26, 27 and 29);
(h) B1 comprises the three CDRs of the heavy chain and/or the three CDRs of the light chain of antibody 1170/1171 (SEQ ID NOs: 32, 41 and 50 and/or SEQ ID NOs: 26, 27 and 61) and B2 comprises the three CDRs of the heavy chain and/or the three CDRs of the light chain of antibody 1208/1135 (SEQ ID NOs: 18, 20 and 23 and/or SEQ ID NOs: 26, 27 and 29);
(i) B1 comprises the three CDRs of the heavy chain and/or the three CDRs of the light chain of antibody 1526/1527 (SEQ ID NOs: 38, 47 and 58 and/or SEQ ID NOs: 26, 27 and 66) and B2 comprises the three CDRs of the heavy chain and/or the three CDRs of the light chain of antibody 1210/1211 (SEQ ID NOs: 18, 20 and 24 and/or SEQ ID NOs: 26, 27 and 30); or
(j) B1 comprises the three CDRs of the heavy chain and/or the three CDRs of the light chain of antibody 1526/1527 (SEQ ID NOs: 38, 47 and 58 and/or SEQ ID NOs: 26, 27 and 66) and B2 comprises the three CDRs of the heavy chain and/or the three CDRs of the light chain of antibody 1208/1135 (SEQ ID NOs: 18, 20 and 23 and/or SEQ ID NOs: 26, 27 and 29)

45. A polypeptide according to any one of the preceding claims wherein:
(a) B1 comprises the heavy chain variable region and/or the light chain variable region of antibody 1170/1167 (SEQ ID NO: 73 and/or SEQ ID NO: 67) and B2 comprises the heavy chain variable region and/or the light chain variable region of antibody 1210/1211 (SEQ ID NO: 9 and/or SEQ ID NO: 11);
(b) B1 comprises the heavy chain variable region and/or the light chain variable region of antibody 1482/1483 (SEQ ID NO: 83 and/or SEQ ID NO: 81) and B2 comprises the heavy chain variable region and/or the light chain variable region of antibody 1210/1211 (SEQ ID NO: 9 and/or SEQ ID NO: 11);
(c) B1 comprises the heavy chain variable region and/or the light chain variable region of antibody 1170/1167 (SEQ ID NO: 73 and/or SEQ ID NO: 67) and B2 comprises the heavy chain variable region and/or the light chain variable region of antibody 1208/1135 (SEQ ID NO: 5 and/or SEQ ID NO: 7);
(d) B1 comprises the heavy chain variable region and/or the light chain variable region of antibody 1482/1483 (SEQ ID NO: 83 and/or SEQ ID NO: 81) and B2 comprises the heavy chain variable region and/or the light chain variable region of antibody 1208/1135 (SEQ ID NO: 5 and/or SEQ ID NO: 7);
(e) B1 comprises the heavy chain variable region and/or the light chain variable region of antibody 1166/1167 (SEQ ID NO: 69 and/or SEQ ID NO: 67) and B2 comprises the heavy chain variable region and/or the light chain variable region of antibody 1210/1211 (SEQ ID NO: 9 and/or SEQ ID NO: 11);
(f) B1 comprises the heavy chain variable region and/or the light chain variable region of antibody 1170/1171 (SEQ ID NO: 73 and/or SEQ ID NO: 71) and B2 comprises the heavy chain variable region and/or the light chain variable region of antibody 1210/1211 (SEQ ID NO: 9 and/or SEQ ID NO: 11);
(g) B1 comprises the heavy chain variable region and/or the light chain variable region of antibody 1166/1167 (SEQ ID NO: 69 and/or SEQ ID NO: 67) and B2 comprises the heavy chain variable region and/or the light chain variable region of antibody 1208/1135 (SEQ ID NO: 5 and/or SEQ ID NO: 7);
(h) B1 comprises the heavy chain variable region and/or the light chain variable region of antibody 1170/1171 (SEQ ID NO: 73 and/or SEQ ID NO: 71) and B2 comprises the heavy chain variable region and/or the light chain variable region of antibody 1208/1135 (SEQ ID NO: 5 and/or SEQ ID NO: 7);
(i) B1 comprises the heavy chain variable region and/or the light chain variable region of antibody 1526/1527 (SEQ ID NO: 99 and/or SEQ ID NO: 97); and B2 comprises the heavy chain variable region and/or the light chain variable region of antibody 1210/1211 (SEQ ID NO: 9 and/or SEQ ID NO: 11);
(j) B1 comprises the heavy chain variable region and/or the light chain variable region of antibody 1526/1527 (SEQ ID NO: 99 and/or SEQ ID NO: 97); and B2 comprises the heavy chain variable region and/or the light chain variable region of antibody 1208/1135 (SEQ ID NO: 5 and/or SEQ ID NO: 7); or
(k) variants of said light chain variable regions and/or said heavy chain variable regions having at least 90% sequence identity thereto.

46. A polypeptide according to any one of the preceding claims wherein:
(a) B1 comprises the three CDRs of the heavy chain and/or the three CDRs of the light chain of antibody 1170/1171 (SEQ ID NOs: 32, 41 and 50 and/or SEQ ID NOs: 26, 27 and 61), or variable regions or antibody chains comprising said CDRs, as defined in claim 42 or 43, and B2 comprises the three CDRs of the heavy chain and/or the three CDRs of the light chain of antibody 1210/1211 (SEQ ID NOs: 18, 20 and 24 and/or SEQ ID NOs: 26, 27 and 30) or variable regions or antibody chains comprising said CDRs, as defined in claim 42 or 43;
(b) B1 comprises the three CDRs of the heavy chain and/or the three CDRs of the light chain of antibody 1170/1171 (SEQ ID NOs: 32, 41 and 50 and/or SEQ ID NOs: 26, 27 and 61) or variable regions or antibody chains comprising said CDRs, as defined in claim 42 or 43, and B2 comprises the three CDRs of the heavy chain and/or the three CDRs of the light chain of antibody 1208/1135 (SEQ ID NOs: 18, 20 and 23 and/or SEQ ID NOs: 26, 27 and 29) or variable regions or antibody chains comprising said CDRs, as defined in claim 42 or 43;
(c) B1 comprises the three CDRs of the heavy chain and/or the three CDRs of the light chain of antibody 1526/1527 (SEQ ID NOs: 38, 47 and 58 and/or SEQ ID NOs: 26, 27 and 66) or variable regions or antibody chains comprising said CDRs, as defined in claim 42 or 43, and B2 comprises the three CDRs of the heavy chain and/or the three CDRs of the light chain of antibody 1210/1211 (SEQ ID NOs: 18, 20 and 24 and/or SEQ ID NOs: 26, 27 and 30) or variable regions or antibody chains comprising said CDRs, as defined in claim 42 or 43; or
(d) B1 comprises the three CDRs of the heavy chain and/or the three CDRs of the light chain of antibody 1526/1527 (SEQ ID NOs: 38, 47 and 58 and/or SEQ ID NOs: 26, 27 and 66) or variable regions or antibody chains comprising said CDRs, as defined in claim 42 or 43, and B2 comprises the three CDRs of the heavy chain and/or the three CDRs of the light chain of antibody 1208/1135 (SEQ ID NOs: 18, 20 and 23 and/or SEQ ID NOs: 26, 27 and 29) or variable regions or antibody chains comprising said CDRs, as defined in claim 42 or 43.

47. A polypeptide according to any one of the preceding claims comprising a heavy chain constant region having an amino acid sequence of SEQ ID NO: 111 and/or a light chain constant region having an amino acid sequence of SEQ ID NO: 112.

48. A polypeptide according to any one of the preceding claims comprising a heavy chain constant region having an amino acid sequence of SEQ ID NO: 103 and/or a light chain constant region having an amino acid sequence of SEQ ID NO: 112.

49. An isolated nucleic acid molecule encoding a bispecific polypeptide according to any one of the preceding claims, or a component polypeptide chain thereof.

50. A nucleic acid molecule according to claim 49 wherein the molecule is a cDNA molecule.

51. A nucleic acid molecule according to claim 49 or 50 encoding an antibody heavy chain or variable region thereof.

52. A nucleic acid molecule according to any one of claims 50 to 51 encoding an antibody light chain or variable region thereof.

53. A vector comprising a nucleic acid molecule according to any one of claims 50 to 52.

54. A vector according to claim 53 wherein the vector is an expression vector.

55. A recombinant host cell comprising a nucleic acid molecule according to any one of claims 47 to 50 or a vector according to claim 53 or 54.

56. A host cell according to claim 55 wherein the host cell is a bacterial cell.

57. A host cell according to claim 55 wherein the host cell is a mammalian cell.

58. A host cell according to claim 55 wherein the host cell is a human cell.

59. A method for producing bispecific polypeptide according to any one of claims 1 to 48, the method comprising culturing a host cell as defined in any of claims 55 to 58 under conditions which permit expression of the bispecific polypeptide or component polypeptide chain thereof.

60. A pharmaceutical composition comprising an effective amount of bispecific polypeptide according to any one of the claims 1 to 48 and a pharmaceutically-acceptable diluent, carrier or excipient.

61. A pharmaceutical composition according to claim 60 adapted for parenteral delivery.

62. A pharmaceutical composition according to claim 60 adapted for intravenous delivery.

63. A bispecific polypeptide according to any one of the claims 1 to 48 for use in medicine.

64. A bispecific polypeptide according to any one of the claims 1 to 48 for use in treating or preventing a neoplastic disorder in a subject.

65. A polypeptide for use according to claim 64 wherein the neoplastic disorder is associated with the formation of solid tumours within the subject's body.

66. A polypeptide for use according to claim 65 wherein the solid tumour is selected from the group consisting of prostate cancer, breast cancer, lung cancer, colorectal cancer, melanomas, bladder cancer, brain/CNS cancer, cervical cancer, oesophageal cancer, gastric cancer, head/neck cancer, kidney cancer, liver cancer, lymphomas, ovarian cancer, pancreatic cancer and sarcomas.

67. A polypeptide for use according to claim 66 wherein the solid tumour is selected from the groups consisting of renal cell carcinoma, colorectal cancer, lung cancer, prostate cancer and breast cancer.

68. A polypeptide for use according to any one of claims 64 to 67 wherein the polypeptide is for use in combination with one or more additional therapeutic agents.

69. A polypeptide for use according to claim 68 wherein the one or more additional therapeutic agents is/are an immunotherapeutic agent that binds a target selected from the group consisting of PD-1/PD-1L, CTLA-4, CD137, CD40, GITR, LAG3, TIM3, CD27, VISTA and KIR.

70. Use of a bispecific polypeptide according to any one of claims 1 to 48 in the preparation of a medicament for treating or preventing a neoplastic disorder in a subject.

71. A use according to claim 70 wherein the neoplastic disorder is associated with the formation of solid tumours within the subject's body.

72. A use according to claim 71 wherein the solid tumour is selected from the group consisting of prostate cancer, breast cancer, lung cancer, colorectal cancer, melanomas, bladder cancer, brain/CNS cancer, cervical cancer, oesophageal cancer, gastric cancer, head/neck cancer, kidney cancer, liver cancer, lymphomas, ovarian cancer, pancreatic cancer and sarcomas.

73. A use according to claim 72 wherein the solid tumour is selected from the groups consisting of renal cell carcinoma, colorectal cancer, lung cancer, prostate cancer and breast cancer.

74. A use according to any one of claims 70 to 73 wherein the polypeptide is for use in combination with one or more additional therapeutic agents.

75. A polypeptide for use according to claim 74 wherein the one or more additional therapeutic agents is/are an immunotherapeutic agent that binds a target selected from the group consisting of PD-1/PD-1L, CTLA-4, CD137, CD40, GITR, LAG3, TIM3, CD27 and KIR.

76. A method for the treatment or diagnosis of a neoplastic disorder in a subject, comprising the step of administering to the subject an effective amount of a bispecific polypeptide according to any one of the claims 1 to 48.

77. A method according to claim 76 wherein the neoplastic disorder is associated with the formation of solid tumours within the subject's body.

78. A method according to claim 77 wherein the solid tumour is selected from the group consisting of prostate cancer, breast cancer, lung cancer, colorectal cancer, melanomas, bladder cancer, brain/CNS cancer, cervical cancer, oesophageal cancer, gastric cancer, head/neck cancer, kidney cancer, liver cancer, lymphomas, ovarian cancer, pancreatic cancer and sarcomas.

79. A method according to claim 78 wherein the solid tumour is selected from the groups consisting of renal cell carcinoma, colorectal cancer, lung cancer, prostate cancer and breast cancer.

80. A method according to any one of claims 76 to 79 wherein the subject is human.

81. A method according to any one of claims 76 to 80 wherein the method comprises administering the bispecific antibody systemically.

82. A method according to any one of claims 76 to 81 further comprising administering to the subject one or more additional therapeutic agents.

83. A method according to any one of claims 76 to 82 wherein the one or more additional therapeutic agents is/are an immunotherapeutic agent that binds a target selected from the group consisting of PD-1/PD-1L, CTLA-4, CD137, CD40, GITR, LAG3, TIM3, CD27 and KIR.

84. A bispecific polypeptide substantially as described herein with reference to the description and figures.

85. A polynucleotide substantially as described herein with reference to the description and figures.

86. A pharmaceutical composition substantially as described herein with reference to the description and figures.

87. Use of a bispecific polypeptide substantially as described herein with reference to the description and figures.

88. A method of treatment substantially as described herein with reference to the description and figures.


SEQUENCE LISTING (132 sequences)

SEQ ID NO: 1 - protein, 121 aa, Artificial Sequence
  1206, heavy chain, VH
EVQLLESGGGLVQPGGSLRLSCAASGFTFSGSSMSWVRQAPGKGLEWVSSIYYSGSGTYYADSVKGRFTISRDNSKNTLYLQMNSLRAEDTAVYYCARYGRNVHPYNLDYWGQGTLVTVSS

SEQ ID NO: 2 - DNA, 363 bp, Artificial Sequence
  1206, heavy chain, VH
gaggtgcagctgttggagagcgggggaggcttggtacagcctggggggtccctgcgcctctcctgtgcagccagcggattcaccttttctggttcttctatgtcttgggtccgccaggctccagggaaggggctggagtgggtctcatctatttactactctggttctggtacatactatgcagactccgtgaagggccggttcaccatctcccgtgacaattccaagaacacgctgtatctgcaaatgaacagcctgcgtgccgaggacacggctgtatattattgtgcgcgctacggtcgtaacgttcatccgtacaacttggactattggggccagggaaccctggtcaccgtctcctca

SEQ ID NO: 3 - protein, 107 aa, Artificial Sequence
  1207, light chain VL
DIQMTQSPSSLSASVGDRVTITCRASQSISSYLNWYQQKPGKAPKLLIYAASSLQSGVPSRFSGSGSGTDFTLTISSLQPEDFATYYCQQGYYYLPTFGQGTKLEIK

SEQ ID NO: 4 - DNA, 321 bp, Artificial Sequence
  1207, light chain VL
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagggttactactacctgcccacttttggccaggggaccaagctggagatcaaa

SEQ ID NO: 5 - protein, 121 aa, Artificial Sequence
  1208, heavy chain VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARSPYYYGANWIDYWGQGTLVTVSS

SEQ ID NO: 6 - DNA, 363 bp, Artificial Sequence
  1208, heavy chain VH
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcagctattagtggtagtggtggtagcacatactatgcagactccgtgaagggccggttcaccatctcccgtgacaattccaagaacacgctgtatctgcaaatgaacagcctgcgtgccgaggacacggctgtatattattgtgcgcgctctccgtactactacggtgctaactggattgactattggggccagggaaccctggtcaccgtctcctca

SEQ ID NO: 7 - protein, 107 aa, Artificial Sequence
  1135, light chain VL
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIK

SEQ ID NO: 8 - DNA, 321 bp, Artificial Sequence
  1135, light chain VL
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagagttacagtaccccttatacttttggccaggggaccaagctggagatcaaa

SEQ ID NO: 9 - protein, 121 aa, Artificial Sequence
  1210, heavy chain VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARYYGGYYSAWMDYWGQGTLVTVSS

SEQ ID NO: 10 - DNA, 363 bp, Artificial Sequence
  1210, heavy chain VH
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcagctattagtggtagtggtggtagcacatactatgcagactccgtgaagggccggttcaccatctcccgtgacaattccaagaacacgctgtatctgcaaatgaacagcctgcgtgccgaggacacggctgtatattattgtgcgcgctactacggtggttactactctgcttggatggactattggggccagggaaccctggtcaccgtctcctca

SEQ ID NO: 11 - protein, 107 aa, Artificial Sequence
  1211, light chain VL
DIQMTQSPSSLSASVGDRVTITCRASQSISSYLNWYQQKPGKAPKLLIYAASSLQSGVPSRFSGSGSGTDFTLTISSLQPEDFATYYCQQTYGYLHTFGQGTKLEIK

SEQ ID NO: 12 - DNA, 321 bp, Artificial Sequence
  1211, light chain VL
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagacttacggttacctgcacacttttggccaggggaccaagctggagatcaaa

SEQ ID NO: 13 - protein, 117 aa, Artificial Sequence
  1212, heavy chain VH
EVQLLESGGGLVQPGGSLRLSCAASGFTFSSYAMSWVRQAPGKGLEWVSYISSYGGYTSYADSVKGRFTISRDNSKNTLYLQMNSLRAEDTAVYYCARYHSGVLDYWGQGTLVTVSS

SEQ ID NO: 14 - DNA, 351 bp, Artificial Sequence
  1212, heavy chain VH
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcatacatttcttcttacggtggttacacatcttatgcagactccgtgaagggccggttcaccatctcccgtgacaattccaagaacacgctgtatctgcaaatgaacagcctgcgtgccgaggacacggctgtatattattgtgcgcgctaccattctggtgttttggactattggggccagggaaccctggtcaccgtctcctca

SEQ ID NO: 15 - protein, 108 aa, Artificial Sequence
  1213, light chain VL
DIQMTQSPSSLSASVGDRVTITCRASQSISSYLNWYQQKPGKAPKLLIYAASSLQSGVPSRFSGSGSGTDFTLTISSLQPEDFATYYCQQYYYHYLLTFGQGTKLEIK

SEQ ID NO: 16 - DNA, 324 bp, Artificial Sequence
  1213, light chain VL
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtactactaccattacctgctcacttttggccaggggaccaagctggagatcaaa

SEQ ID NO: 17 - protein, 8 aa, Artificial Sequence
  1206 VH CDR H1
GFTFSGSS

SEQ ID NO: 18 - protein, 8 aa, Artificial Sequence
  1208 VH CDR H1 or 1210 VH CDR H1 or 1212 VH CDR H1
GFTFSSYA

SEQ ID NO: 19 - protein, 8 aa, Artificial Sequence
  1206 VH CDR H2
IYYSGSGT

SEQ ID NO: 20 - protein, 8 aa, Artificial Sequence
  1208 VH CDR H2 or 1210 VH CDR H2
ISGSGGST

SEQ ID NO: 21 - protein, 8 aa, Artificial Sequence
  1212 VH CDR H2
ISSYGGYT

SEQ ID NO: 22 - protein, 14 aa, Artificial Sequence
  1206 VH CDR H3
ARYGRNVHPYNLDY

SEQ ID NO: 23 - protein, 14 aa, Artificial Sequence
  1208 VH CDR H3
ARSPYYYGANWIDY

SEQ ID NO: 24 - protein, 14 aa, Artificial Sequence
  1210 VH CDR H3
ARYYGGYYSAWMDY

SEQ ID NO: 25 - protein, 10 aa, Artificial Sequence
  1212 VH CDR H3
ARYHSGVLDY

SEQ ID NO: 26 - protein, 6 aa, Artificial Sequence
  1207 VL CDR L1 or 1135 VL CDR L1 or 1211 VL CDR L1 or 1213 VL CDR L1 or VL 1167 L CDR1 or VL 1171 L CDR1 or VL 1135 L CDR1 or VL 1483 L CDR1 or VL 1515 L CDR1 or VL 1525 L CDR1 or VL 1527 L CDR1
QSISSY

SEQ ID NO: 27 - skipped

SEQ ID NO: 28 - protein, 9 aa, Artificial Sequence
  1207 VL CDR L3
QQGYYYLPT

SEQ ID NO: 29 - protein, 9 aa, Artificial Sequence
  1135 VL CDR L3
QQSYSTPYT

SEQ ID NO: 30 - protein, 9 aa, Artificial Sequence
  1211 VL CDR L3
QQTYGYLHT

SEQ ID NO: 31 - protein, 10 aa, Artificial Sequence
  1213 VL CDR L3
QQYYYHYLLT

SEQ ID NO: 32 - protein, 8 aa, Artificial Sequence
  VH 1166 H CDR1 or VH 1170 H CDR1
GFTFGGYY

SEQ ID NO: 33 - protein, 8 aa, Artificial Sequence
  VH 1164 H CDR1
GFTFYGSS

SEQ ID NO: 34 - protein, 8 aa, Artificial Sequence
  VH 1168 H CDR1
GFTFSGSS

SEQ ID NO: 35 - protein, 8 aa, Artificial Sequence
  VH 1482 H CDR1 or VH 1490 H CDR1 or VH 1520 H CDR1
GFTFSSYA

SEQ ID NO: 36 - protein, 8 aa, Artificial Sequence
  VH 1514 H CDR1
GFTFGYYY

SEQ ID NO: 37 - protein, 8 aa, Artificial Sequence
  VH 1524 H CDR1
GFTFGSYY

SEQ ID NO: 38 - protein, 8 aa, Artificial Sequence
  VH 1526 H CDR1
GFTFSGYS

SEQ ID NO: 39 - protein, 8 aa, Artificial Sequence
  VH 1542 H CDR1
GFTFGSSS

SEQ ID NO: 40 - protein, 8 aa, Artificial Sequence
  VH 1166 H CDR2 or VH 1520 H CDR2
ISGSGGST

SEQ ID NO: 41 - protein, 8 aa, Artificial Sequence
  VH 1170 H CDR2
IPGSGGST

SEQ ID NO: 42 - protein, 8 aa, Artificial Sequence
  VH 1164 H CDR2
IYSSGGYT

SEQ ID NO: 43 - protein, 8 aa, Artificial Sequence
  VH 1168 H CDR2 or VH 1490 H CDR2
ISYYGGYT

SEQ ID NO: 44 - protein, 8 aa, Artificial Sequence
  VH 1482 H CDR2
ISYYSGYT

SEQ ID NO: 45 - protein, 8 aa, Artificial Sequence
  VH 1514 H CDR2
ISSYGSYT

SEQ ID NO: 46 - protein, 8 aa, Artificial Sequence
  VH 1524 H CDR2
IGSYYGYT

SEQ ID NO: 47 - protein, 8 aa, Artificial Sequence
  VH 1526 H CDR2
IGYSGYGT

SEQ ID NO: 48 - protein, 9 aa, Artificial Sequence
  VH 1542 H CDR2
IGYYSYSTS

SEQ ID NO: 49 - protein, 10 aa, Artificial Sequence
  VH 1166 H CDR3
ARYDYASMDY

SEQ ID NO: 50 - protein, 10 aa, Artificial Sequence
  VH 1170 H CDR3
ARYDYYWMDY

SEQ ID NO: 51 - protein, 11 aa, Artificial Sequence
  VH 1164 H CDR3
ARGVPHGYFDY

SEQ ID NO: 52 - protein, 11 aa, Artificial Sequence
  VH 1168 H CDR3
ARYFPHYYFDY

SEQ ID NO: 53 - protein, 9 aa, Artificial Sequence
  VH 1482 H CDR3
ARGYGYLDY

SEQ ID NO: 54 - protein, 11 aa, Artificial Sequence
  VH 1490 H CDR3
ARYYPHHYIDY

SEQ ID NO: 55 - protein, 14 aa, Artificial Sequence
  VH 1514 H CDR3
ARSGYSNWANSFDY

SEQ ID NO: 56 - protein, 17 aa, Artificial Sequence
  VH 1520 H CDR3
ARYYYSHGYYVYGTLDY

SEQ ID NO: 57 - protein, 10 aa, Artificial Sequence
  VH 1524 H CDR3
ARHDYGALDY

SEQ ID NO: 58 - protein, 15 aa, Artificial Sequence
  VH 1526 H CDR3
ARYYFHDYAAYSLDY

SEQ ID NO: 59 - protein, 11 aa, Artificial Sequence
  VH 1542 H CDR3
ARGYPHHYFDY

SEQ ID NO: 60 - protein, 10 aa, Artificial Sequence
  VL 1167 L CDR3
QQYYWYGLST

SEQ ID NO: 61 - protein, 10 aa, Artificial Sequence
  VL 1171 L CDR3
QQGHGSYPHT

SEQ ID NO: 62 - protein, 9 aa, Artificial Sequence
  VL 1135 L CDR3
QQSYSTPYT

SEQ ID NO: 63 - protein, 8 aa, Artificial Sequence
  VL 1483 L CDR3
QQYGSLLT

SEQ ID NO: 64 - protein, 9 aa, Artificial Sequence
  VL 1515 L CDR3
QQGDYTLFT

SEQ ID NO: 65 - protein, 10 aa, Artificial Sequence
  VL 1525 L CDR3
QQYGPSGLFT

SEQ ID NO: 66 - protein, 10 aa, Artificial Sequence
  VL 1527 L CDR3
QQYGSDSLLT

SEQ ID NO: 67 - protein, 108 aa, Artificial Sequence
  1167, light chain VL
DIQMTQSPSSLSASVGDRVTITCRASQSISSYLNWYQQKPGKAPKLLIYAASSLQSGVPSRFSGSGSGTDFTLTISSLQPEDFATYYCQQYYWYGLSTFGQGTKLEIK

SEQ ID NO: 68 - DNA, 324 bp, Artificial Sequence
  1167, light chain VL
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtactactggtacggtctgtccacttttggccaggggaccaagctggagatcaaa

SEQ ID NO: 69 - protein, 117 aa, Artificial Sequence
  1166, heavy chain VH
EVQLLESGGGLVQPGGSLRLSCAASGFTFGGYYMSWVRQAPGKGLEWVSAISGSGGSTYYADSVKGRFTISRDNSKNTLYLQMNSLRAEDTAVYYCARYDYASMDYWGQGTLVTVSS

SEQ ID NO: 70 - DNA, 351 bp, Artificial Sequence
  1166, heavy chain VH
gaggtgcagctgttggagagcgggggaggcttggtacagcctggggggtccctgcgcctctcctgtgcagccagcggattcacctttggtggttactacatgtcttgggtccgccaggctccagggaaggggctggagtgggtctcagctattagtggtagtggtggtagcacatactatgcagactccgtgaagggccggttcaccatctcccgtgacaattccaagaacacgctgtatctgcaaatgaacagcctgcgtgccgaggacacggctgtatattattgtgcgcgctacgactacgcttctatggactattggggccagggaaccctggtcaccgtctcctca

SEQ ID NO: 71 - protein, 108 aa, Artificial Sequence
  1171, light chain VL
DIQMTQSPSSLSASVGDRVTITCRASQSISSYLNWYQQKPGKAPKLLIYAASSLQSGVPSRFSGSGSGTDFTLTISSLQPEDFATYYCQQGHGSYPHTFGQGTKLEIK

SEQ ID NO: 72 - DNA, 324 bp, Artificial Sequence
  1171, light chain VL
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagggtcatggttcttacccgcacacttttggccaggggaccaagctggagatcaaa

SEQ ID NO: 73 - protein, 117 aa, Artificial Sequence
  1170, heavy chain VH
EVQLLESGGGLVQPGGSLRLSCAASGFTFGGYYMSWVRQAPGKGLEWVSYIPGSGGSTYYADSVKGRFTISRDNSKNTLYLQMNSLRAEDTAVYYCARYDYYWMDYWGQGTLVTVSS

SEQ ID NO: 74 - DNA, 351 bp, Artificial Sequence
  1170, heavy chain VH
gaggtgcagctgttggagagcgggggaggcttggtacagcctggggggtccctgcgcctctcctgtgcagccagcggattcacctttggtggttactacatgtcttgggtccgccaggctccagggaaggggctggagtgggtctcatacattcctggttctggtggttctacatactatgcagactccgtgaagggccggttcaccatctcccgtgacaattccaagaacacgctgtatctgcaaatgaacagcctgcgtgccgaggacacggctgtatattattgtgcgcgctacgactactactggatggactattggggccagggaaccctggtcaccgtctcctca

SEQ ID NO: 75 - protein, 107 aa, Artificial Sequence; identical to SEQ ID NO: 7
  1135, light chain VL

SEQ ID NO: 76 - DNA, 321 bp, Artificial Sequence; identical to SEQ ID NO: 8
  1135, light chain VL

SEQ ID NO: 77 - protein, 118 aa, Artificial Sequence
  1164, heavy chain VH
EVQLLESGGGLVQPGGSLRLSCAASGFTFYGSSMYWVRQAPGKGLEWVSGIYSSGGYTSYADSVKGRFTISRDNSKNTLYLQMNSLRAEDTAVYYCARGVPHGYFDYWGQGTLVTVSS

SEQ ID NO: 78 - DNA, 354 bp, Artificial Sequence
  1164, heavy chain VH
gaggtgcagctgttggagagcgggggaggcttggtacagcctggggggtccctgcgcctctcctgtgcagccagcggattcaccttttacggttcttctatgtactgggtccgccaggctccagggaaggggctggagtgggtctcaggtatttactcttctggtggttacacatcttatgcagactccgtgaagggccggttcaccatctcccgtgacaattccaagaacacgctgtatctgcaaatgaacagcctgcgtgccgaggacacggctgtatattattgtgcgcgcggtgttcctcatggttactttgactattggggccagggaaccctggtcaccgtctcctca

SEQ ID NO: 79 - protein, 118 aa, Artificial Sequence
  1168, heavy chain VH
EVQLLESGGGLVQPGGSLRLSCAASGFTFSGSSMSWVRQAPGKGLEWVSSISYYGGYTYYADSVKGRFTISRDNSKNTLYLQMNSLRAEDTAVYYCARYFPHYYFDYWGQGTLVTVSS

SEQ ID NO: 80 - DNA, 354 bp, Artificial Sequence
  1168, heavy chain VH
gaggtgcagctgttggagagcgggggaggcttggtacagcctggggggtccctgcgcctctcctgtgcagccagcggattcacctttagtggttcttctatgtcttgggtccgccaggctccagggaaggggctggagtgggtctcatctatttcttactacggtggttacacatactatgcagactccgtgaagggccggttcaccatctcccgtgacaattccaagaacacgctgtatctgcaaatgaacagcctgcgtgccgaggacacggctgtatattattgtgcgcgctacttcccgcattactactttgactattggggccagggaaccctggtcaccgtctcctca

SEQ ID NO: 81 - protein, 106 aa, Artificial Sequence
  1483, light chain VL
DIQMTQSPSSLSASVGDRVTITCRASQSISSYLNWYQQKPGKAPKLLIYAASSLQSGVPSRFSGSGSGTDFTLTISSLQPEDFATYYCQQYGSLLTFGQGTKLEIK

SEQ ID NO: 82 - DNA, 318 bp, Artificial Sequence
  1483, light chain VL
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtacggttctctgctcacttttggccaggggaccaagctggagatcaaa

SEQ ID NO: 83 - protein, 116 aa, Artificial Sequence
  1482, heavy chain VH
EVQLLESGGGLVQPGGSLRLSCAASGFTFSSYAMSWVRQAPGKGLEWVSYISYYSGYTYYADSVKGRFTISRDNSKNTLYLQMNSLRAEDTAVYYCARGYGYLDYWGQGTLVTVSS

SEQ ID NO: 84 - DNA, 348 bp, Artificial Sequence
  1482, heavy chain VH
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcatacatttcttactactctggttacacatactatgcagactccgtgaagggccggttcaccatctcccgtgacaattccaagaacacgctgtatctgcaaatgaacagcctgcgtgccgaggacacggctgtatattattgtgcgcgcggttacggttacttggactattggggccagggaaccctggtcaccgtctcctca

SEQ ID NO: 85 - protein, 118 aa, Artificial Sequence
  1490, heavy chain VH
EVQLLESGGGLVQPGGSLRLSCAASGFTFSSYAMSWVRQAPGKGLEWVSGISYYGGYTYYADSVKGRFTISRDNSKNTLYLQMNSLRAEDTAVYYCARYYPHHYIDYWGQGTLVTVSS

SEQ ID NO: 86 - DNA, 354 bp, Artificial Sequence
  1490, heavy chain VH
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcaggtatttcttactacggtggttacacatactatgcagactccgtgaagggccggttcaccatctcccgtgacaattccaagaacacgctgtatctgcaaatgaacagcctgcgtgccgaggacacggctgtatattattgtgcgcgctactacccgcatcattacattgactattggggccagggaaccctggtcaccgtctcctca

SEQ ID NO: 87 - protein, 107 aa, Artificial Sequence
  1515, light chain VL
DIQMTQSPSSLSASVGDRVTITCRASQSISSYLNWYQQKPGKAPKLLIYAASSLQSGVPSRFSGSGSGTDFTLTISSLQPEDFATYYCQQGDYTLFTFGQGTKLEIK

SEQ ID NO: 88 - DNA, 321 bp, Artificial Sequence
  1515, light chain VL
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagggtgattacactctgttcacttttggccaggggaccaagctggagatcaaa

SEQ ID NO: 89 - protein, 121 aa, Artificial Sequence
  1514, heavy chain VH
EVQLLESGGGLVQPGGSLRLSCAASGFTFGYYYMSWVRQAPGKGLEWVSGISSYGSYTYYADSVKGRFTISRDNSKNTLYLQMNSLRAEDTAVYYCARSGYSNWANSFDYWGQGTLVTVSS

SEQ ID NO: 90 - DNA, 363 bp, Artificial Sequence
  1514, heavy chain VH
gaggtgcagctgttggagagcgggggaggcttggtacagcctggggggtccctgcgcctctcctgtgcagccagcggattcacctttggttactactacatgtcttgggtccgccaggctccagggaaggggctggagtgggtctcaggtatttcttcttacggtagttacacatactatgcagactccgtgaagggccggttcaccatctcccgtgacaattccaagaacacgctgtatctgcaaatgaacagcctgcgtgccgaggacacggctgtatattattgtgcgcgctctggttactctaactgggctaactcttttgactattggggccagggaaccctggtcaccgtctcctca

SEQ ID NO: 91 - protein, 124 aa, Artificial Sequence
  1520, heavy chain VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARYYYSHGYYVYGTLDYWGQGTLVTVSS

SEQ ID NO: 92 - DNA, 372 bp, Artificial Sequence
  1520, heavy chain VH
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcagctattagtggtagtggtggtagcacatactatgcagactccgtgaagggccggttcaccatctcccgtgacaattccaagaacacgctgtatctgcaaatgaacagcctgcgtgccgaggacacggctgtatattattgtgcgcgctactactactctcatggttactacgtttacggtactttggactattggggccagggaaccctggtcaccgtctcctca

SEQ ID NO: 93 - protein, 108 aa, Artificial Sequence
  1525, light chain VL
DIQMTQSPSSLSASVGDRVTITCRASQSISSYLNWYQQKPGKAPKLLIYAASSLQSGVPSRFSGSGSGTDFTLTISSLQPEDFATYYCQQYGPSGLFTFGQGTKLEIK

SEQ ID NO: 94 - DNA, 324 bp, Artificial Sequence
  1525, light chain VL
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtacggtccgtctggtctgttcacttttggccaggggaccaagctggagatcaaa

SEQ ID NO: 95 - protein, 117 aa, Artificial Sequence
  1524, heavy chain VH
EVQLLESGGGLVQPGGSLRLSCAASGFTFGSYYMGWVRQAPGKGLEWVSSIGSYYGYTYYADSVKGRFTISRDNSKNTLYLQMNSLRAEDTAVYYCARHDYGALDYWGQGTLVTVSS

SEQ ID NO: 96 - DNA, 351 bp, Artificial Sequence
  1524, heavy chain VH
gaggtgcagctgttggagagcgggggaggcttggtacagcctggggggtccctgcgcctctcctgtgcagccagcggattcacctttggttcttactacatgggttgggtccgccaggctccagggaaggggctggagtgggtctcatctattggttcttactacggttacacatactatgcagactccgtgaagggccggttcaccatctcccgtgacaattccaagaacacgctgtatctgcaaatgaacagcctgcgtgccgaggacacggctgtatattattgtgcgcgccatgactacggtgctttggactattggggccagggaaccctggtcaccgtctcctca

SEQ ID NO: 97 - protein, 108 aa, Artificial Sequence
  1527, light chain VL
DIQMTQSPSSLSASVGDRVTITCRASQSISSYLNWYQQKPGKAPKLLIYAASSLQSGVPSRFSGSGSGTDFTLTISSLQPEDFATYYCQQYGSDSLLTFGQGTKLEIK

SEQ ID NO: 98 - DNA, 324 bp, Artificial Sequence
  1527, light chain VL
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtacggttctgattctctgctcacttttggccaggggaccaagctggagatcaaa

SEQ ID NO: 99 - protein, 122 aa, Artificial Sequence
  1526, heavy chain VH
EVQLLESGGGLVQPGGSLRLSCAASGFTFSGYSMYWVRQAPGKGLEWVSGIGYSGYGTYYADSVKGRFTISRDNSKNTLYLQMNSLRAEDTAVYYCARYYFHDYAAYSLDYWGQGTLVTVSS

SEQ ID NO: 100 - DNA, 366 bp, Artificial Sequence
  1526, heavy chain VH
gaggtgcagctgttggagagcgggggaggcttggtacagcctggggggtccctgcgcctctcctgtgcagccagcggattcaccttttctggttactctatgtactgggtccgccaggctccagggaaggggctggagtgggtctcaggtattggttactctggttacggtacatactatgcagactccgtgaagggccggttcaccatctcccgtgacaattccaagaacacgctgtatctgcaaatgaacagcctgcgtgccgaggacacggctgtatattattgtgcgcgctactacttccatgactacgctgcttactctttggactattggggccagggaaccctggtcaccgtctcctca

SEQ ID NO: 101 - protein, 118 aa, Artificial Sequence
  1542, heavy chain VH
EVQLLESGGGLVQPGGSLRLSCAASGFTFGSSSMYWVRQAPGKGLEWVSGIGYYSYSTSYADSVKGRFTISRDNSKNTLYLQMNSLRAEDTAVYYCARGYPHHYFDYWGQGTLVTVSS

SEQ ID NO: 102 - DNA, 354 bp, Artificial Sequence
  1542, heavy chain VH
gaggtgcagctgttggagagcgggggaggcttggtacagcctggggggtccctgcgcctctcctgtgcagccagcggattcacctttggttcttcttctatgtactgggtccgccaggctccagggaaggggctggagtgggtctcaggtattggttactactcttactctacatcttatgcagactccgtgaagggccggttcaccatctcccgtgacaattccaagaacacgctgtatctgcaaatgaacagcctgcgtgccgaggacacggctgtatattattgtgcgcgcggttacccgcatcattactttgactattggggccagggaaccctggtcaccgtctcctca

SEQ ID NO: 103 - protein, 330 aa, Artificial Sequence
  IgG1 LALA-sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 11 aa, Artificial Sequence
  Linker
SGGGGSGGGGS

SEQ ID NO: 105 - protein, 13 aa, Artificial Sequence
  Linker
SGGGGSGGGGSAP

SEQ ID NO: 106 - protein, 5 aa, Artificial Sequence
  Linker
NFSQP

SEQ ID NO: 107 - protein, 5 aa, Artificial Sequence
  Linker
KRTVA

SEQ ID NO: 108 - protein, 8 aa, Artificial Sequence
  Linker
GGGSGGGG

SEQ ID NO: 109 - protein, 10 aa, Artificial Sequence
  Linker
GGGGSGGGGS

SEQ ID NO: 110 - protein, 15 aa, Artificial Sequence
  Linker
GGGGSGGGGSGGGGS

SEQ ID NO: 111 - protein, 330 aa, Artificial Sequence [sequence omitted]

SEQ ID NO: 112 - protein, 107 aa, Artificial Sequence [sequence omitted]

SEQ ID NO: 113 - protein, 327 aa, Artificial Sequence
  Modified IgG4 heavy chain constant region sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRYTQKSLSLSLGK

SEQ ID NO: 114 - protein, 327 aa, Artificial Sequence
  Modified IgG4 heavy chain constant region sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 115 - protein, 327 aa, Artificial Sequence
  Wild type IgG4 heavy chain constant region sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 116 - protein, 18 aa, Artificial Sequence
  Linker
GSTSGSGKPGSGEGSTKG

SEQ ID NO: 117 - protein, 15 aa, Artificial Sequence
  Linker
THTCPPCPEPKSSDK

SEQ ID NO: 118 - protein, 4 aa, Artificial Sequence
  Linker
GGGS

SEQ ID NO: 119 - protein, 13 aa, Artificial Sequence
  Linker
EAAKEAAKGGGGS

SEQ ID NO: 120 - protein, 8 aa, Artificial Sequence
  Linker
EAAKEAAK

SEQ ID NO: 121 - protein, 4 aa, Artificial Sequence
  Linker [(SG)m where m=2]
SGSG

SEQ ID NO: 122 - protein, 6 aa, Artificial Sequence
  Linker [(SG)m where m=3]
SGSGSG

SEQ ID NO: 123 - protein, 8 aa, Artificial Sequence
  Linker [(SG)m where m=4]
SGSGSGSG

SEQ ID NO: 124 - protein, 10 aa, Artificial Sequence
  Linker [(SG)m where m=5]
SGSGSGSGSG

SEQ ID NO: 125 - protein, 12 aa, Artificial Sequence
  Linker [(SG)m where m=6]
SGSGSGSGSGSG

SEQ ID NO: 126 - protein, 14 aa, Artificial Sequence
  Linker [(SG)m where m=7]
SGSGSGSGSGSGSG

SEQ ID NO: 127 - protein, 8 aa, Artificial Sequence
  anti-OX40 heavy chain CDR1 consensus sequence
  VARIANT 5: Gly or Tyr or Ser
  VARIANT 6: Gly or Tyr or Ser
  VARIANT 7: Tyr or Ser
  VARIANT 8: Tyr or Ser or Ala
GFTFXXXX

SEQ ID NO: 128 - protein, 8 aa, Artificial Sequence
  anti-OX40 heavy chain CDR2 consensus sequence
  VARIANT 2: Gly or Tyr or Ser or Thr
  VARIANT 3: Gly or Ser or Tyr
  VARIANT 4: Ser or Tyr
  VARIANT 5: Gly or Ser or Tyr
  VARIANT 6: Gly or Ser or Tyr
  VARIANT 7: Gly or Ser or Tyr
IXXXXXXT

SEQ ID NO: 129 - protein, 17 aa, Artificial Sequence
  anti-OX40 heavy chain CDR3 consensus sequence
  VARIANT 3: Gly or Tyr or Ser or His
  VARIANT 4: Gly or Tyr or Phe or Val or Asp
  VARIANT 5: Gly or Tyr or Pro or Phe
  VARIANT 6: no amino acid or His or Ser
  VARIANT 7: no amino acid or Asn or Asp or His
  VARIANT 8: no amino acid or Tyr or Gly
  VARIANT 9: no amino acid or Tyr
  VARIANT 10: no amino acid or Tyr
  VARIANT 11: no amino acid or Trp or Ala or Val
  VARIANT 12: no amino acid or Ala or Tyr
  VARIANT 13: no amino acid or Asp or Ala or Tyr or Gly or His or Asn
  VARIANT 14: Tyr or Ser or Trp or Ala or Thr
  VARIANT 15: Leu or Met or Ile or Phe
ARXXXXXXXXXXXXXDY

SEQ ID NO: 130 - protein, 10 aa, Artificial Sequence
  anti-OX40 heavy chain CDR3 preferred 10-amino acid consensus sequence
  VARIANT 3: Tyr or His
  VARIANT 6: Ala or Tyr or Gly
  VARIANT 7: Ser or Trp or Ala
  VARIANT 8: Met or Leu
ARXDYXXXDY

SEQ ID NO: 131 - protein, 11 aa, Artificial Sequence
  anti-OX40 heavy chain CDR3 preferred 11-amino acid consensus sequence
  VARIANT 3: Gly or Tyr
  VARIANT 4: Val or Phe or Tyr
  VARIANT 7: Gly or Tyr or His
  VARIANT 9: Phe or Ile
ARXXPHXYXDY

SEQ ID NO: 132 - protein, 10 aa, Artificial Sequence
  anti-OX40 light chain CDR3 consensus sequence
  VARIANT 3: Ser or Tyr or Gly
  VARIANT 4: no amino acid or Tyr or His or Gly
  VARIANT 5: no amino acid or Ser or Tyr or Gly or Asp
  VARIANT 6: Ser or Tyr or Gly or Asp
  VARIANT 7: Ser or Tyr or Gly or Thr
  VARIANT 8: Pro or Leu
  VARIANT 9: Tyr or Ser or His or Leu or Phe
QQXXXXXXXT